(19) United States
(12) Patent Application Publication
(10) Pub. No.: US 2022/0202845 A1
(43) Pub. Date: Jun. 30, 2022

(54) METHODS AND COMPOSITIONS FOR TREATING CANCER

(71) Applicant: Fred Hutchinson Cancer Research Center, Seattle, WA (US)

(72) Inventors: Robert Bradley, Seattle, WA (US); Guo-Liang Chew, Seattle, WA (US); Amy Campbell, Seattle, WA (US); Stephen Tapscott, Seattle, WA (US)

(21) Appl. No.: 17/264,713

(22) PCT Filed: Jul. 25, 2019
(86) PCT No.: PCT/US2019/043396

§ 371 Date: Jan. 29, 2021

Related U.S. Application Data
(60) Provisional application No. 62/857,632, filed on Jun. 5, 2019.
(60) Provisional application No. 62/711,662, filed on Jul. 30, 2018.

(51) Int. Cl. A61K 31/713; A61K 31/506; A61K 45/06; G01N 33/574

(52) U.S. Cl. CPC A61K 31/713; G01N 33/574; A61K 45/06; A61K 31/506

(57) ABSTRACT

DUX4 can be used as a biomarker for predicting a patient's response to immunotherapy, and inhibition of DUX4 can be used in therapeutic methods for treating DUX4+ cancers. Accordingly, aspects of the disclosure relate to a method for treating cancer in a patient comprising administering a DUX4 inhibitor to the patient. Further aspects of the disclosure relate to a method for treating a DUX4+ cancer in a patient, the method comprising administering a checkpoint inhibitor to the patient. Other aspects relate to a composition comprising a DUX4 inhibitor and a checkpoint inhibitor.

### CROSS-REFERENCE TO RELATED APPLICATIONS

This application claims priority to U.S. Provisional Application Ser. No. 62/711,662, filed Jul. 30, 2018, and 62/857,632, filed Jun. 5, 2019, each of which is incorporated here by reference in its entirety.

### STATEMENT REGARDING FEDERALLY FUNDED RESEARCH

This invention was made with government support under NS069539 and AR045203 awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 1896-P45USPNP_Seq_List_FINAL_20220211_ST25.txt. The text file is 50 KB; was created on Feb. 11, 2022, and is being submitted via EFS-Web with the filing of the specification.

### BACKGROUND OF THE INVENTION

### 1. Field of the Invention

This invention relates to the field of molecular biology and medicine.

### 2. Background

Immune checkpoint blockade therapies, which act on T cell inhibitory receptors including CTLA-4 and PD-1, induce durable responses across diverse cancers. However, a majority of patients do not respond to these therapies. Furthermore, initially responsive cancers may eventually relapse. The efficacy of immune checkpoint blockade relies upon cytotoxic T cell recognition of antigens presented by MHC Class I on malignant cells. As a consequence, genetic lesions that suppress antigen presentation or blunt tumor-immune interactions can permit malignant cells to evade cytotoxic T cells.

Stratifying patients into those that are likely and unlikely to respond to checkpoint blockade therapy, based on one or more biomarkers, will provide for more effective and therapeutic treatment method for patients, since patients can be provided with the most effective therapy before further spreading of the disease. Furthermore, there is a need in the art for more effective therapies for those patients that are predicted to be non-responsive to checkpoint inhibitor therapy.

### SUMMARY OF THE INVENTION

The inventors found that DUX4 was a novel regulator of antigen presentation and immune modulation. DUX4 can be used as a biomarker for predicting a patient's response to immunotherapy, and inhibition of DUX4 can be used in therapeutic methods for treating DUX4+ cancers. Accordingly, aspects of the disclosure relate to a method for treating cancer in a patient comprising administering a DUX4 inhibitor to the patient. Further aspects of the disclosure relate to a method for treating a DUX4+ cancer in a patient, the method comprising administering a checkpoint inhibitor to the patient. Further aspects of the disclosure relate to a method for treating a DUX4+ cancer in a patient, the method comprising administering an immunotherapy to the patient. Other aspects relate to a composition comprising a DUX4 inhibitor and a checkpoint inhibitor.

In some embodiments, the methods comprise or further comprise the administration of a DUX4 inhibitor. In some embodiments, the DUX4 inhibitor comprises a nucleic acid inhibitor, an antibody, a polypeptide, or a molecular inhibitor. In some embodiments, the DUX4 inhibitor comprises an antisense oligonucleotide, small interfering RNA (siRNA), short hairpin RNA (shRNA), double-stranded RNA, an antisense oligonucleotide, a ribozyme, or combinations thereof.

In some embodiments, the DUX4 inhibitor comprises a molecular inhibitor. In some embodiments, the DUX4 molecular inhibitor is selected from a Bromodomain and Extra-Terminal motif (BET) inhibitor, a CDK7 inhibitor, a Wnt pathway agonist, a beta-2 adrenergic receptor agonist, a p38 inhibitor, a phosphodiesterase (PDE) inhibitor, a cAMP analog, and combinations thereof. In some embodiments, the DUX4 inhibitor comprises a BET inhibitor that is a selective inhibitor of BRD4, a selective inhibitor of BRD2, or a broad spectrum inhibitor. In some embodiments, the DUX4 inhibitor comprises a BET inhibitor selected from JQ1, PFI-1, I-BET-762, I-BET-151, RVX-208, CPI-0610, and combinations thereof. In some embodiments, the DUX4 inhibitor comprises a CDK7 inhibitor. Exemplary CDK7 inhibitors include LDC4297, THZ1, BS-181, and those disclosed in US 2016/0264552, US 2017/0174692, US 2017/0183355, US 2016/0264554, and US 2016/0122323 (all of which are incorporated herein by reference). In some embodiments, the inhibitor comprises THZ1. In some embodiments, the DUX4 inhibitor comprises a beta-2 andrenergic agonist selected from formoterol, albuterol, CGP20712, CI118,551, clenbuterol, and combinations thereof. In some embodiments, the beta-2 andrenergic agonist comprises bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, olodaterol, or combinations thereof.

In some embodiments, the DUX4 inhibitor comprises a PDE inhibitor selected from ibudilast, crisaborole, and combinations thereof. In some embodiments, the DUX4 inhibitor comprises 8-Bromoadenosine 3′,5′-cyclic monophosphate or protein kinase A.

In some embodiments, the DUX4 inhibititor is a p38 inhibitor. In some embodiments, the inhibitor of p38 is an inhibitor of p38α and p38β such as a selective inhibitor of p38 α. In some embodiments, the inhibitor of p38 modulates the expression of DUX4. The inhibitor of p38 may not inhibit the MK2 pathway. In other embodiments, the inhibitor of p38 does not inhibit either p38δ or p38γ. The inhibitor of p38 may be selected from acumapimod, ARRY-371797, BMS-582949, dilmapimod, dorimapimod, losmapimod, LY222820, LY3007113, pamapimod, PH-797804, SB202190, SB203580, talmapimod, VX-702, and VX-745.

In some embodiments, the DUX4 inhibitor comprises a neutralizing antibody. In some embodiments, the DUX4 inhibitor comprises a DUX4-s polypeptide.

In some embodiments, the cancer comprises cutaneous squamous-cell carcinoma, non-colorectal and colorectal gastrointestinal cancer, Merkel-cell carcinoma, anal cancer, cervical cancer, hepatocellular cancer, urothelial cancer, melanoma, lung cancer, non-small cell lung cancer, small cell lung cancer, head and neck cancer, kidney cancer, bladder cancer, Hodgkin's lymphoma, pancreatic cancer, or skin cancer. In some embodiments, the cancer comprises one described herein. In some embodiments, the cancer comprises metastatic cancer. In some embodiments, the cancer comprises a solid tumor. In some embodiments, the cancer excludes hematological cancers. In some embodiments, the cancer excludes one or more cancers described herein.

In some embodiments, the cancer comprises a DUX4+ cancer. In some embodiments, the cancer comprises a high-expressing DUX4+ cancer. In some embodiments, the cancer comprises a DUX4+ expression level that is at least that of the expression level in the patients whose expression level is in the top 5, 10, 15, 20, 25, 30, 35, 40, 45, 50, 55, 60, 65, 70, 75, 80, 85, or 90% (or any derivable range therein). In some embodiments, the patient has been determined to have a DUX4+ cancer. In some embodiments, the patient has been determined to have a high-expressing DUX4+ cancer. In some embodiments, the patient is determined to have a DUX4 expression level that is at least that of the expression level in the patients whose expression level is in the top 5, 10, 15, 20, 25, 30, 35, 40, 45, 50, 55, 60, 65, 70, 75, 80, 85, or 90% (or any derivable range therein). In some embodiments the DUX4+ cancer that has expression level of 0.1-100, 0.5-100, 0.5-20, 1-20, 2-20, or 0.5-50 transcripts per million (TPM). In some embodiments, the DUX4+ cancer or high-expressing DUX4+ cancer and/or DUX4 regulated gene is at least, at most, or exactly 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 22, 24, 26, 28, 30, 32, 34, 46, 48, 40, 42, 44, 46, 48, 50, 55, 60, 65, 70, 75, 80, 85, 90, 100, 125, 150, 175, 200, 250, 300, 400, or 400 (or any derivable range therein) TPM. Methods for determining TPM are known in the art. For example Li et al., Bioinformatics. 2010 Feb. 15; 26(4): 493-500, which is herein incorporated by reference, describes TPM determination.

In some embodiments, the patient has been determined to have differential expression of one or more DUX4-regulated genes. In some embodiments, the expression of the DUX4-regulated gene is increased or decreased relative to a reference sample. In some embodiments, the reference sample comprises a normal, or non-cancerous cell. In some embodiments, the DUX4-regulated gene is increased relative to a reference or control level of expression. In some embodiments, the DUX4-regulated gene is decreased relative to a reference or control level of expression. In some embodiments, the DUX4-regulated gene is one known in the art or described herein. In some embodiments, the DUX4-regulated gene comprises one described as a biomarker in International Application Publications: WO2013019623, WO2012024535, WO2013192185, and WO2015143062, which are herein incorporated by reference for all purposes. In some embodiments, the patient has been diagnosed with recurrent cancer. In some embodiments, the DUX4-regulated genes comprise one or more of CCNA1 (ENSG00000133101), CTD-2611012.2 (ENSG00000229292), DUXA (ENSG00000258873), DUXB, HNRNPCL1 (ENSG00000179172), KDM4E (ENSG00000235268), KHDC1L (ENSG00000256980), KLF17 (ENSG00000171872), LEUTX (ENSG00000213921), MBD3L2 (ENSG00000230522), MBD3L3 (ENSG00000182315), PRAMEF1 (ENSG00000116721), PRAMEF11 (ENSG00000204513), PRAMEF12 (ENSG00000116726), PRAMEF13 (ENSG00000204495), PRAMEF14 (ENSG00000204481), PRAMEF15 (ENSG00000157358), PRAMEF2 (ENSG00000120952), PRAMEF20 (ENSG00000204478), PRAMEF4 (ENSG00000243073), PRAMEF5 (ENSG00000204502), PRAMEF6 (ENSG00000232423), PRAMEF7 (ENSG00000204510), PRAMEF9 (ENSG00000204501), RFPL1 (ENSG00000128250), RFPL2 (ENSG00000128253), RFPL4A (ENSG00000223638), RFPL4B (ENSG00000251258), SLC34A2 (ENSG00000157765), TRIM43 (ENSG00000144015), TRIM48 (ENSG00000150244), TRIM49 (ENSG00000168930), TRIM49B (ENSG00000182053), TRIM49C (ENSG00000204449), TRIM51BP (ENSG00000204455), TRIM53AP (ENSG00000225581), TRIM53BP (ENSG00000166013), W12-2994D6.2 (ENSG00000229571), W12-3308P17.2 (ENSG00000239810), ZSCAN4 (ENSG00000180532), ZSCANSB (ENSG00000197213), ZSCANSC (ENSG00000204532), and ZSCANSD (ENSG00000267908). In some embodiments, the DUX4-regulated gene comprises a repetitive element. In some embodiments, the repetive element comprises one or more of HERVL, HSATII, LSAU, MLT2A1, MLT2A1, SVA_D, and SVA_E. In some embodiments, the DUX4 regulated gene(s) comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70 (or any derivable range therein of a DUX4 regulated gene described herein and/or of CCNA1 (ENSG00000133101), CTD-2611012.2 (ENSG00000229292), DUXA (ENSG00000258873), DUXB, HNRNPCL1 (ENSG00000179172), KDM4E (ENSG00000235268), KHDC1L (ENSG00000256980), KLF17 (ENSG00000171872), LEUTX (ENSG00000213921), MBD3L2 (ENSG00000230522), MBD3L3 (ENSG00000182315), PRAMEF1 (ENSG00000116721), PRAMEF11 (ENSG00000204513), PRAMEF12 (ENSG00000116726), PRAMEF13 (ENSG00000204495), PRAMEF14 (ENSG00000204481), PRAMEF15 (ENSG00000157358), PRAMEF2 (ENSG00000120952), PRAMEF20 (ENSG00000204478), PRAMEF4 (ENSG00000243073), PRAMEF5 (ENSG00000204502), PRAMEF6 (ENSG00000232423), PRAMEF7 (ENSG00000204510), PRAMEF9 (ENSG00000204501), RFPL1 (ENSG00000128250), RFPL2 (ENSG00000128253), RFPL4A (ENSG00000223638), RFPL4B (ENSG00000251258), SLC34A2 (ENSG00000157765), TRIM43 (ENSG00000144015), TRIM48 (ENSG00000150244), TRIM49 (ENSG00000168930), TRIM49B (ENSG00000182053), TRIM49C (ENSG00000204449), TRIM51BP (ENSG00000204455), TRIM53AP (ENSG00000225581), TRIM53BP (ENSG00000166013), W12-2994D6.2 (ENSG00000229571), W12-3308P17.2 (ENSG00000239810), ZSCAN4 (ENSG00000180532), ZSCANSB (ENSG00000197213), ZSCANSC (ENSG00000204532), ZSCANSD (ENSG00000267908), HERVL, HSATII, LSAU, MLT2A1, MLT2A1, SVA_D, and SVA_E

In some embodiments, the method further comprises determining the level of expression of DUX4 in a sample from the patient. In some embodiments, the method further comprises determining the level of expression of a DUX4-regulated gene in a sample from the patient. In some embodiments, the sample comprises cancerous tissues.

In some embodiments, the patient has been previously treated with a prior cancer therapy. In some embodiments, the patient was determined to be resistant, poorly responsive, or non-responsive to the prior cancer therapy. In some embodiments, the prior cancer therapy comprises an immunotherapy. In some embodiments, the method further comprises administration of an additional cancer therapy. In some embodiments, the additional therapy comprises chemotherapy, radiation, surgery, or immunotherapy. In some embodiments, the additional cancer therapy is administered prior to, after, or concurrently with the DUX4 inhibitor. In some embodiments, the immunotherapy comprises a checkpoint inhibitor therapy. In some embodiments, the checkpoint inhibitor is selected from an antagonist of CTLA-4, PD-1, PD-L1, and combinations thereof. In some embodiments, the checkpoint inhibitor is selected from an antagonist of CTLA-4, PD-1, PD-L1, PD-L2, LAG-3, TIM-3, VISTA, TIGIT, IDO1, or combinations thereof. In some embodiments, the checkpoint inhibitor is selected from Ipilmumab, Nivolumab, Pembrolizumab, BMS-936559, MSB0010718C, MPDL3280A, MedI-4736, pidilizumab, AMP-224, RG7446, Atezolizumab, Ipilimumab, Durvalumab, LY3321367 MBG453, TSR-022, JNJ-61610588, and combinations thereof.

In some embodiments, the cancer comprises a solid tumor. In some embodiments, the tumor comprises DUX4+ cancer cells on the periphery of the tumor. The term periphery refers to cells at the interface between cancerous tissues and non-cancerouse tissues or fluid.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D. Identification of genes with cancer-specific expression. (A) Overview of data sources and the inventors' strategy for identifying cancer-specific gene expression. The inventors compared the expression of each gene in cancer samples (TCGA) to its corresponding expression in peritumoral samples (TCGA) and normal tissue from healthy individuals (Illumina Body Map 2.0, Human Proteome Map, and Genotype-Tissue Expression Project, GTEx). The inventors defined the cancer specificity score for each gene as the logarithm of the fractions of cancer samples and types in which the gene was expressed divided by the fractions of peritumoral samples and normal tissues in which the gene was expressed. (B) Ranked plot of cancer specificity scores of coding genes, restricted to genes that are not expressed in all tissue types. The double homeobox genes DUX4, DUXA, and DUXB are highlighted. (C) Expression of cancer-specific genes across cancer types and samples. Each point corresponds to a gene highlighted in red in (B). y axis, number of cancer types (TCGA primary site) with at least one DUX4+ sample; x axis, total number of DUX4+ samples, irrespective of cancer type. (D) DUX4 mRNA levels in DUX4+ cancer samples and during early embryogenesis (Hendrickson et al., 2017). TPM, transcripts per million.

FIG. 2A-H. DUX4 is expressed as a full-length mRNA in diverse solid cancers. (A) Schematic of DUX4 isoform that encodes the full-length transcription factor. Dark grey, sequence encoding the DNA-binding homeodomains (HB1 and HB2); light grey, sequence encoding the C-terminal activation domain (Activation Domain). (B-D) Read coverage of the DUX4 isoform illustrated in (A) in embryos (Hendrickson et al., 2017), representative DUX4+ solid cancers, and B-ALL with DUX4-IGH translocations. Gray shaded box, open reading frame. Plot based on image from IGV. (E) Numbers of RNA-seq reads containing DUX4's polyadenylation site in its third exon (Exon 3 PAS), where each point corresponds to a single sample and is classified based on whether reads from that sample arose from a “permissive” 4qA161 allele or a “non-permissive” (10qA or 4qB) allele. The analyzed data are from poly(A)-selected libraries. ***, p<0.001 by the one-sided Mann-Whitney U test. (F) DUX4 mRNA levels (TPM) in samples from testicular germ cell tumors, segregated by DPPA2 and DPPA4 expression. <25th and >75th indicate the bottom and top quartiles. ***/****, p<0.001/0.0001 by the one-sided Mann-Whitney U test. Error bars, standard deviations estimated by bootstrapping. TPM, transcripts per million. (G) Association between predicted loss-of-function mutations affecting known or likely repressors of DUX4 expression with increased DUX4 mRNA levels. Shade of grey indicate p-values computed with a one-sided Mann-Whitney U test. See also FIG. 8D. (H) qRT-PCR measurement of PRPF8, DUX4, ZSCAN4, and TRIM43 mRNA levels following transfection of a pool of four PRPF8-targeting siRNAs or a control non-targeting siRNA into myoblasts isolated from a healthy (MB2401) or FSHD (MB073) individual. The FSHD myoblasts have a permissive genetic background that potentiates DUX4 expression. Error bars, standard deviation across biological replicates.

FIG. 3A-B. DUX4 drives an embryonic gene expression program in cancer. (A) Differential gene expression induced by endogenous or ectopic DUX4. Each point represents mRNA levels for a single gene in samples with (y axis) or without (x axis) DUX4 expression. Plots illustrate comparisons between cleavage-stage embryos and zygotes, which have high and low DUX4 expression (left panel), iPSCs with or without DUX4 induction (center panel), and myoblasts with or without DUX4 induction (right panel) (Feng et al., 2015; Hendrickson et al., 2017). Red, high-confidence list of DUX4-induced genes identified by intersecting the sets of up-regulated genes in all three illustrated comparisons. TPM, transcripts per million. (B) Expression of high-confidence DUX4 targets in DUX4+ cancers. Heat map illustrates the expression of each DUX4 target (rows) in each individual DUX4+ sample (columns) relative to the median expression across all DUX4− samples from that cancer type. The LSAU repetitive element corresponds to the beta satellite repeat, which is a multicopy genomic element like DUX4. Approximately 55% of LSAU's 2,759 nt consensus sequence overlaps with part of DUX4's consensus sequence, so its expression is invariably higher in DUX4+ samples when expression is quantified via short-read sequencing.

FIG. 4A-E. DUX4 is associated with cancer immune evasion. (A) Gene Ontology (GO) terms that were enriched for genes that were differentially expressed in DUX4+ versus DUX4− samples across multiple cancer types. Plot is restricted to the child-most GO terms with a False Discovery Rate (FDR)≤0.01 after Benjamini-Hochberg correction. Circle areas are proportional to −log 10 (FDR). (B) Estimated CD8+ T cell infiltration in DUX4− and DUX4+ cancers, where infiltration was estimated with the TIMER method (Li et al., 2017). */**, p<0.05/0.01 by the one-sided Mann-Whitney U test. (C) Mean estimated cytolytic activity in DUX4− and DUX4+ cancers. Cytolytic activity was estimated as the geometric mean of GZMA and PRF1 gene expression (Rooney et al., 2015). Error bars, standard deviations estimated by bootstrapping. (D) Mean expression of canonical MHC Class I genes, where the mean was computed over all DUX4− and DUX4+ cancers for each type. Error bars, standard deviations estimated by bootstrapping. TPM, transcripts per million. (E) As (D), but for the illustrated datasets. Feng et al. 2015 introduced DUX4 via lentivirus into 54-1 and MB135 myoblasts (Feng et al., 2015), Rickard et al. 2015 sorted DUX4− and DUX4+ myoblasts following induction of differentiation for myoblasts that spontaneously express DUX4 (Rickard et al., 2015), and Eidahl et al. 2016 transfected DUX4-expressing plasmids into WS236 myoblasts (Eidahl et al., 2016). n, number of replicates.

FIG. 5A-K. DUX4 blocks interferon-γ-mediated up-regulation of MHC Class I-dependent antigen presentation. (A) Mean expression of genes encoding the illustrated components of the interferon-γ signaling pathway, where the mean was computed over all DUX4− and DUX4+ cancers for each type. Error bars, standard deviations estimated by bootstrapping. TPM, transcripts per million. (B) As (A), but for the illustrated datasets. (C) As (A), but illustrating gene expression during preimplantation embryonic development. (D-I) Immunoblots probing MHC Class I, DUX4, and GAPDH protein following treatment of the indicated cell lines, each of which was engineered to contain a doxycycline-inducible DUX4 expression construct, with interferon-γ (IFN-γ) and/or doxycycline (Dox) to induce DUX4. γ-MHC I, pan-MHC Class I probe. (J-K) Levels of MHC Class I on the cell surface following treatment of the indicated cell lines with interferon-γ (IFN-γ) and/or doxycycline (Dox) to induce DUX4. Cell surface levels of MHC Class I were probed with a pan-MHC Class I antibody.

FIG. 6A-D DUX4 expression is associated with resistance to immune checkpoint blockade. (A) Fractions of DUX4 target genes (Table S3) that are expressed in pre-treatment biopsies taken from metastatic melanoma patients who received anti-CTLA-4 (Van Allen et al., 2015) or anti-PD-1 (Hugo et al., 2016) therapy. Clinical responses were classified in the original studies according to RECIST (anti-CTLA-4) (Eisenhauer et al., 2009) or irRECIST (anti-PD-1) (Wolchok et al., 2009). p-value computed with the Wilcoxon rank-sum test. (B) Progression-free survival for patients treated with anti-CTLA-4 whose pre-treatment biopsies fell within the top or bottom terciles of DUX4 target gene expression. p-value computed with the log-rank test. (C) As (B), but for overall survival. (D) As (B), but for the cohort of patients treated with anti-PD-1.

FIG. 7A-B. Cancer specificity of non-coding genes. (A) As FIG. 1A, but for non-coding genes that are not expressed in all tissue types. (B) As FIG. 1C, but for non-coding genes.

FIG. 8A-D. DUX4 is expressed as a full-length mRNA in solid cancers. (A) Schematic of DUX4 isoform that encodes the full-length transcription factor. Dark grey, sequence encoding the DNA-binding homeodomains (HB1 and HB2); light grey, sequence encoding the C-terminal activation domain (Activation Domain). (B) As FIG. 2C, but including data from additional representative DUX4+ samples from each analyzed cancer type. (C) Counts of RNA-seq reads mapping uniquely to the DUX4 mRNA encoding the full-length transcription factor (DUX4fl) or the DUX4C mRNA (DUX4c). Plot restricted to DUX4+ samples in cancer types with ≥5 DUX4+ samples. (D) Median DUX4 mRNA levels (TPM) in cancer samples with (Mut) or without (WT) predicted loss-of-function mutations affecting the indicated genes. */**/***, p<0.05/0.01/0.001 by the one-sided Mann-Whitney U test. See also FIG. 2G. Error bars, standard deviations estimated by bootstrapping. TPM, transcripts per million.

FIG. 9A-C. DUX4 expression is associated with decreased immune activity. (A) Scatter plots of median gene expression of DUX4− and DUX4+ samples. Differentially expressed genes that are up- and down-regulated in DUX4+ samples in multiple (≥8) cancers are indicated in red and blue, respectively. (B) Mean levels of DUXB mRNA following treatment of MB135 myoblasts engineered to contain a doxycycline-inducible DUXB expression construct with vehicle (Veh, water) or doxycycline (Dox) for 24 hours. Error bars, upper and lower values from two biological replicates. (C) Differentially expressed genes in myoblasts treated with vehicle or doxycycline to induce DUXB expression. Lightest grey, high-confidence DUX4-induced genes (from FIG. 3A). Darkest grey, DUXB-induced genes based on this analysis, defined as genes exhibiting a fold-change of >2 and associated Bayes factor >10 in both biological replicates. TPM, transcripts per million.

FIG. 10A-F. DUX4+ cancers exhibit reduced immune cell infiltration. (A) Estimated infiltration of different immune cell types in DUX4− and DUX4+ cancers, where infiltration was estimated with the TIMER method (Li et al., 2017). */**/***, p<0.05/0.01/0.001 by the one-sided Mann-Whitney U test. (B) Mean mRNA levels of the indicated markers of CD8+ T cells in DUX4− and DUX4+ cancers. Error bars, standard deviation of the mean estimated by bootstrapping. (C) As (B), but illustrating NK cell markers. (D) As (B), but illustrating GZMA and PRF1 expression. See also FIG. 4C. (E) As (B), but illustrating FOXP3 expression. (F) Estimated infiltration of regulatory T cells in DUX4− and DUX4+ cancers as estimated by CIBERSORT (Newman et al., 2015). */**/***, p<0.05/0.01/0.001 by the one-sided Mann-Whitney U test.

FIG. 11A-H. DUX4 blocks interferon-g-mediated induction of MHC Class I. (A) Read coverage from DUX4 ChIP-seq experiments following acute DUX4 expression in cultured myoblasts (Geng et al., 2012). Red blocks indicate peaks called with MACS (Zhang et al., 2008). n, number of replicates. (B) Levels of B2M, HLA-A, HLA-B, and HLA-C mRNA following treatment of MB135iDUX4 with IFN-g and/or doxycycline (Dox) to induce DUX4. Error bars, standard deviation across biological replicates. (C) Levels of DUX4, ZSCAN4, and GAPDH protein following treatment of MB135iDUX4 cells with interferon-g (IFN-g) and/or doxycycline (Dox) to induce DUX4. (D) Levels of MHC Class I and GAPDH protein following treatment of the indicated parental cell lines (without an inducible DUX4 construct) with interferon-g (IFN-g) and/or doxycycline (Dox). a-MHC I, pan-MHC Class I probe. These data serve as a control for FIG. 5D-I to confirm that doxycycline treatment alone does not block interferon-g-mediated induction of MHC Class I. (E) As FIG. 5J, but including the no antibody control (No Ab). (F) As FIG. 5K, but including the no antibody control (No Ab). (G) Mean CXCL9 and CXCL10 mRNA levels in DUX4− and DUX4+ cancers. Error bars, standard deviation of the mean estimated by bootstrapping. (H) As (B), but illustrating CXCL9 and 10 mRNA levels.

FIG. 12. siRNA-mediated inhibition of DUX4 protein expression blocks the DUX4-suppression of the induction of MHC Class I by IFN [symbol] . MB135iDUX4ca myoblasts were transfected with siCTRL or siCADUX4 siRNAs. After 28 hours, doxycycline was added to induce DUX4. IFN [symbol] was added four hours after DOX to induce immune response. The cells were harvested 14 hours after IFN [symbol] addition. The top shows an immunoblot probing MHC class I, DUX4, and GAPDH protein following treatment of MB135 myoblasts engineered to contain a doxycycline-inducible, codon-altered DUX4 expression construct (MB135_iDUX4ca) with non-targeting control (CTRL) or siRNAs targeting the codon altered DUX4 RNA (siCADUX4), doxycycline (Dox) to induce DUX4, and interferon-γ (IFN-γ). α-MHC I, pan-MHC class I probe. The bottom shows a quantification of the immunoblot.

FIG. 13A-C. CDK7 inhibitor THZ1 reduces DUX4 expression in FSHD cells and cancer cells. (A) Differentiating MB200 FSHD2 patient-derived muscle cells, (B) SuSa testicular teratocarcinoma cells, or (C) KLE endometrial adenocarcinoma cells were treated with vehicle (−, DMSO) or varying concentrations of THZ1, as indicated, for 24 hours (A) or 20 hours (B-C) and cultures analyzed for DUX4 mRNA levels by RT-qPCR. The CDK7 inhibitor THZ1 was identified as a drug that inhibits DUX4 expression in FSHD cells. Data are represented as relative expression (means, with error bars indicating standard deviation across three biological replicates) with expression in the absence of inhibitor set to one. FSHD muscle cells utilize DUX4 exon 3, while SuSa cells and KLE cells use DUX4 exon 3b.

### DESCRIPTION OF ILLUSTRATIVE EMBODIMENTS

The inventors found that DUX4, an early embryonic transcription factor that is normally silenced in somatic tissues, is re-expressed in many solid cancer types. DUX4 re-expression in cancer results in suppression of antigen presentation, immune evasion, and resistance to immune checkpoint blockade. Therefore, DUX4 is useful not only as a biomarker for identifying patients that may respond to immunotherapies, but also as a target to increase the effectiveness of immunotherapies.

### I. DEFINITIONS

“DUX4” means any nucleic acid or protein of DUX4. “DUX4 nucleic acid” means any nucleic acid encoding DUX4 protein. In certain embodiments, DUX4 nucleic acid comprises GENBANK Accession No. FJ439133.1, SEQ ID NO: 3. For example, in certain embodiments, a DUX4 nucleic acid includes a DNA sequence encoding DUX4, an RNA sequence transcribed from DNA encoding DUX4 (including genomic DNA comprising introns and exons), including a non-protein encoding (i.e., non-coding) RNA sequence, and an mRNA sequence encoding DUX4. In some embodiments, DUX4 comprises SEQ ID NO:1 or 2.

The term “nucleic acid inhibitor” refers to a nucleic acid that is capable of inhibiting the expression of DUX4. The nucleic acid may be RNA, DNA, double-stranded, or single-stranded, unless otherwise specified.

The term “antibody” refers to an antibody of any isotype, unless specified otherwise. The antibody may have any heavy or light chain type, unless specified otherwise. In some embodiments, the antibody may be IgA, IgD, IgE, IgG, IgM, IgA1, IgA2, IgG1, IgG2, IgG3, or IgG4. In some embodiments, the antibody comprises an alpha, gamma, epsilon, delta, or mu heavy chain. In some embodiments, the light chain comprises a lambda or kappa light chain. In some embodiments, the inhibitor comprises a fragment of the antibody. In some embodiments, the inhibitor, such as the DUX4 inhibitor comprises a fragment of a DUX4 antibody, wherein the fragment comprises a variable heavy chain region and a variable light chain region. In some embodiments, the DUX4 inhibitor comprises a DUX4-binding antibody fragment. Examples include small chain variable fragments (scFv), Fab fragments, and single-domain antibodies (sdAb).

A “molecular inhibitor” refers to an organic or inorganic compound or polypeptide. In some embodiments, the molecular inhibitor is an organic compound. In some embodiments, a molecular inhibitor excludes antibodies and/or nucleic acids. In some embodiments, the molecular inhibitor is a small molecule. A small molecule is a low molecular weight (<900 daltons) organic compound, with a size on the order of 1 nm.

A neutralizing antibody refers to an antibody that interferes with the biological activity of its antigen. For example, a DUX4 neutralizing antibody inhibits DUX4 by interfering with its biological function, such as it's function as a transcriptional activator and more specifically it's ability to bind DNA.

A “DUX4+ cancer” refers to a cancer in which at least a portion of these cells express DUX4. In some embodiments, a DUX4+ cancer is one is which at least 0.1, 0.2, 0.3, 0.4, 0.5, 0.6, 0.7, 0.8, 0.9, 1, 1.5, 2, 2.5, 3, 3.5, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, or 95% of the cells in a sample express DUX4. In some embodiments, a DUX4+ cancer which has been demonstrated to have DUX4+ cells in a cancerous sample from the patient.

The term “high-expressing” refers to a patient population determined to have high expression of DUX4. Methods of stratifying patients and determining cut-off values based on protein expression are known in the art and described herein (ROC analysis, for example). In some embodiments, the term “high-expressing” refers to a cut-off value, wherein any value above the cut-off value represents an expression level found in the top 5, 10, 15, 20, 25, 30, 35, 40, 45, 50, 55, 60, 65, 70, 75, 80, or 85% of patient's with the greatest amount of DUX4 expression. In some embodiments, the term “low-expressing” refers to a cut-off value, wherein any value below the cut-off value represents an expression level found in the bottom 5, 10, 15, 20, 25, 30, 35, 40, 45, 50, 55, 60, 65, 70, 75, 80, or 85% of patient's with the lowest amount of DUX4 expression that is greater than zero DUX4 expression.

The term “determined to have” refers to a patient population that has been tested and reported as having a certain outcome, such as a DUX4+ cancer.

“Double-stranded siRNA” means any duplex RNA structure comprising two anti-parallel and substantially complementary nucleic acid strands. In certain embodiments, Double-stranded siRNA comprise a sense strand and an antisense strand, wherein the antisense strand is complementary to a target nucleic acid.

“Complementarity” means the capacity for pairing between nucleobases of a first nucleic acid and a second nucleic acid.

Contiguous nucleobases” means nucleobases immediately adjacent to each other.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Modified internucleoside linkage” refers to a substitution or any change from a naturally occurring internucleoside bond (i.e., a phosphodiester internucleoside bond).

“Modified nucleoside” means a nucleoside having, independently, a modified sugar moiety and/or modified nucleobase.

“Nucleobase” means a heterocyclic moiety capable of pairing with a base of another nucleic acid.

“Nucleobase sequence” means the order of contiguous nucleobases independent of any sugar, linkage, and/or nucleobase modification.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Target gene” refers to a gene encoding a target.

“Target nucleic acid” refers to a nucleic acid, the modulation of which is desired.

The term “identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI. In some embodiments, a nucleic acid or amino acid sequence has at least 80%, or at least about 85%, or at least about 90%, or at least about 95%, or at least about 98% or at least about 99% sequence identity to the nucleic acid or amino acid sequence of the disclosure.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

### II. DUX4 INHIBITORS

A DUX4 inhibitor may refer to any member of the class of compound or agents having an IC50 of 100 μM or lower concentration for a DUX4 activity, for example, at least or at most or about 200, 100, 80, 50, 40, 20, 10, 5, 1 μM, 100, 10, 1 nM or lower concentration (or any range or value derivable therefrom) or any compound or agent that inhibits the expression of DUX4.

A. Molecular Inhibitors

Exemplary molecular inhibitors of DUX4 such as molecular compounds known in the art and described herein.

In some embodiments, the DUX4 inhibitor comprises a Extra-Terminal motif (BET) inhibitor. BET inhibitors are a class of drugs with anti-cancer, immunosuppressive, and other effects in clinical trials. BET inhibitors include molecules that are inhibitors of BET proteins such as BRD2, BRD3, BRD4, and BRDT. These inhibitors may prevent protein-protein interaction between BET proteins and acetylated histones and transcription factors. Examples of BET inhibitors include: JQ1, PFI-1, I-BET 151 (GSK1210151A), I-BET 762 (GSK525762), OTX-015, TEN-010 (Tensha therapeutics), CPI-203, RVX-208 (Resverlogix Corp), LY294002, MK-8628 (Merck/Mitsubishi Tanabe), BMS-986158 (Bristol-Myers Squibb), INCB54329 (Incyte Pharmaceuticals), ABBV-075 (AbbVie, also called ABV-075), CPI-0610 (Constellation Pharmaceuticals/Roche), FT-1101 (Forma Therapeutics/Celgene), GS-5829 (Gilead Sciences), and PLX51107 (Daiichi Sankyo). In some embodiments, the BET inhibitor is selected from JQ1, PFI-1, I-BET-762, I-BET-151, RVX-208, CPI-0610, and combinations thereof. BET inhibitors are also described in, for example US20150087636 and Campbell et al., Skeletal Muscle (2017) 7:16, which are herein incorporated by reference for all purposes.

In some embodiments, the DUX4 inhibitor comprise a Wnt pathyway agonists. Examples include recombinant 2-amino-4-[3,4-(methylenedioxy)benzyl-amino]-6-(3-methoxyphenyl)pyrimidin-e, GSK-3-beta inhibitors (e.g., CHIR99021, BIO, SB-216763, and those described by Meijer et al. Trends Pharmacol Sci (2004) 25(9):471-480), (2′Z,3′E)-6-Bromoindirubin-3′-oxime, 5-(Furan-2-yl)-N-(3-(1H-imidazol-1-yl)propyl)-1,2-oxazole-3-carboxamide, tankyrase inhibitors (such as e.g., XAV939, WIKI4, JW55, IWR1) and beta-catenin activators (e.g., SKL2001, deoxycholic acid (DCA)).

In some embodiments, the DUX4 inhibitor comprises a beta-2 andrenergic receptor agonist. Examples include formoterol, albuterol, CGP20712, CI118,551, clenbuterol, and combinations thereof.

In some embodiments, the DUX4 inhibitor comprises a PDE inhibitor. Examples of PDE inhibitors include ibudilast and crisaborole. In some embodiments, the DUX4 inhibitor comprises a cAMP analog. In some embodiments, the cAMP analog comprises 8-Bromoadenosine 3′,5′-cyclic monophosphate. In some embodiments, the DUX4 inhibitor comprises protein kinase A.

In some embodiments, the DUX4 inhibitor is selected from bromosparine, clenbuterol hydrochloride, tulobuterol hydrochloride, albuterol, fenoterol hydrobromide, fluticasone, nylidrin, sirolimus, terbutaline hemisulfate, vinblastine, dequalinium, bisoctrizole, isoetharine, penicillamine, benzethonium chloride, cetylpyridinium chloride, epinephrine bitartrate, phenylephrine, piromidic acid, acenocoumarol, atorvastatin, cresopirine, dicoumarol, thiostrepton, thimerosal, ethylnorepinephrine, fluvoxamine, clozapine, adrenalone hydrochloride, butamben, thonzonium, mebendazole, puromycin, colforsin, dimercaprol, benzalkonium, norepinephrine, polymyxin B, oxedrine (synephrine), nonoxynol-9, papaverine, pentamidine, triamcinolone, dichlorephen, ebselen, minocycline, inosine, sulbentine, artesunate, broxaldine, broxyquinoline, meclocycline, sennoside A, and combinations thereof.

BET inhibitors are also described in, for example US20150087636, US20180050043, and Campbell et al., Skeletal Muscle (2017) 7:16, which are herein incorporated by reference for all purposes. Other DUX4 inhibitors are described in Cruz et al., Protein kinase A activation inhibits DUX4 gene expression in myotubes from patients with facioscapulohumeral muscular dystrophy. Journal of Biological Chemistry (2018), Published on Jun. 13, 2018 as Manuscript RA118.002633, which is incorporated by reference.

As used herein, the abbreviation “p38” refers to any of the p38 mitogen-activated protein (MAP) kinases. p38 is a mammalian protein kinase involved cell proliferation, cell death and response to extracellular stimuli. Several p38 MAP kinases have been identified, including p38-α (also known as MAPK14), p38-β (also known as MAPK11), p38-γ (also known as MAPK12/ERK6), and and p38-δ (also known as MAPK13/SAPK4). The nucleic acid sequences of the genes encoding p38, including, but not limited to, the nucleic acid sequences of the open reading frames of the genes, are known in the art. The amino acid sequences of p38 polypeptides and proteins, including, but not limited to, the amino acid sequences of the human p38 polypeptides and proteins, are known in the art. The accession number of the nucleic acid sequence of Mus musculus p38-α (MAPK14) is NM_011951.3 and the accession number of the nucleic acid sequence of human p38-α (MAPK14) is NM_001315.2. The accession number of the amino acid sequence of Mus musculus p38-a (MAPK14) is NP 036081.1 and the accession number of the amino acid sequence of human p38-α (MAPK14) is NP 001306.1. For additional information on p38, see Marber et al., 2011; see also Kostenko et al., 2011 and Cuadrado et al., 2010, all of which are incorporated by reference herein.

Both pan p38 inhibitors and specific inhibitors of one isoform of p38 are contemplated herein and may be used in the compositions and methods of the disclosure. A non-limiting selections selection of different inhibitors of p38 or related pathways are shown in Table 1 below.

TABLE 1
Potency of Non-limiting List of Inhibitors in differentiating
cultures of FSHD1 and FHD2 muscle cells.

| Inhibitor | Mechanism/Selectivity |
|---|---|
| Acumapimod | p38α/β |
| ARRY-371797 | p38α |
| BMS-582949 | p38α5X selective over p38β |
| Dilmapimod | p38α/β |
| Dorimapimod | p38α20X selective over B-Raf |
| eFT-508 | MNK 1/2 |
| Losmapimod | p38α/β |
| LY2228820 | p38α/β |
| LY3007113 | p38 |
| Pamapimod | p38α34X selective over p38β |
| PF-3644022 | MAPKAPK2 (MK2) |
| PH-797804 | p38α4X selective over p38β |
| SB202190 | p38α/β |
| SB203580 | p38α/β |
| Talmapimod | p38α10X selective over p38β |
| VX-702 | p38α14X selective over p38β |
| VX-745 | p38α22X selective over p38β |

Additional p38 inhibitors which may be used herein include those described Lee et al., Immunopharmacology, 47(2-3):185-201, 2000, Kumar et al., Nat. Rev. Drug Discov., 2(9):717-726, 2003, Gangwal et al, Current Topics in Medicinal Chemistry, 13(9): 1015-1035, 2013, Karcher and Laufer, Curr. Top. Med. Chem., 9(7):655-676, 2009, Yong et al., Expert Opin. Investig. Drugs, 18(12): 1893-1905, 2009, Fisk et al., Am. J. Cardiovasc. Drugs, 14:155-165, 2014, Norman, Expert Opin. Investig. Drugs, 24(3):383-392, 2015, WO 2003/068747, WO 2003/093248, US 2005/0020540, US 2006/0122221, US 2004/0267012, WO 2005/012241, WO 2004/010995, WO 2005/073189, US 2004/0102636, US 2004/0132729, US 2005/0020590, WO 1999/032463, US 2003/0232831, WO 2000/012497, US 2002/0118671, WO 2000/012074, WO 2010/067131, WO 1999/000357, US 2004/0254236, U.S. Pat. No. 5,945,418, US 2009/0143422, US 2009/0136596, US 2007/0185175, US 2009/0118272, US 2007/0213300, US 2008/0171741, US 2011/0190292, WO 2009/155388, US 2011/0077243, WO 2012/031057, and WO 2009/155388, the entirety of which are herein by incorporated by reference.

B. Nucleic Acid Inhibitors

Inhibitory nucleic acids or any ways of inhibiting gene expression of DUX4 known in the art are contemplated in certain embodiments. Examples of an inhibitory nucleic acid include but are not limited to siRNA (small interfering RNA), short hairpin RNA (shRNA), double-stranded RNA, an antisense oligonucleotide, a ribozyme and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. The nucleic acid may have nucleotides of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 or any range derivable therefrom.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Particularly, an inhibitory nucleic acid may be capable of decreasing the expression of DUX4 by at least 10%, 20%, 30%, or 40%, more particularly by at least 50%, 60%, or 70%, and most particularly by at least 75%, 80%, 90%, 95% or more or any range or value in between the foregoing.

In further embodiments, there are synthetic nucleic acids that are DUX4 inhibitors. An inhibitor may be between 17 to 25 nucleotides in length and comprises a 5′ to 3′ sequence that is at least 90% complementary to the 5′ to 3′ sequence of a mature DUX4 mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature DUX4 mRNA, particularly a mature, naturally occurring mRNA, or the sequence of the DUX4 pre-mRNA transcript, or the sequence of the D4Z4 macrosatellite repeat encompassing the DUX4 transcribed region. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In certain embodiments provided are DUX4 inhibitory nucleic acid compounds targeted to a human DUX4 nucleic acid. In certain embodiments, the human DUX4 nucleic acid is the sequence set forth in GENBANK Accession No. NM_001306068.1 (variant 1):

(SEQ ID NO: 1)
atggccctcccgacaccctcggacagcaccctccccgcggaagcccggg
gacgaggacggcgacggagactcgtttggaccccgagccaaagcgaggc
cctgcgagcctgctagagcggaacccgtacccgggcatcgccaccagag
aacggctggcccaggccatcggcattccggagcccagggtccagatttg
gtttcagaatgagaggtcacgccagctgaggcagcaccggcgggaatct
cggccctggcccgggagacgcggcccgccagaaggccggcgaaagcgga
ccgccgtcaccggatcccagaccgccctgctcctccgagcctttgagaa
ggatcgctttccaggcatcgccgcccgggaggagctggccagagagacg
ggcctcccggagtccaggattcagatctggtttcagaatcgaagggcca
ggcacccgggacagggtggcagggcgcccgcgcaggcaggcggcctgtg
cagcgcggcccccggcgggggtcaccctgctccctcgtgggtcgccttc
gcccacaccggcgcgtggggaacggggcttcccgcaccccacgtgccct
gcgcgcctggggctctcccacagggggctttcgtgagccaggcagcgag
ggccgcccccgcgctgcagcccagccaggccgcgccggcagaggggatc
tcccaacctgccccggcgcgcggggatttcgcctacgccgccccggctc
ctccggacggggcgctctcccaccctcaggctcctcgctggcctccgca
cccgggcaaaagccgggaggaccgggacccgcagcgcgacggcctgccg
ggcccctgcgcggtggcacagcctgggcccgctcaagcggggccgcagg
gccaaggggtgcttgcgccacccacgtcccaggggagtccgtggtgggg
ctggggccggggtccccaggtcgccggggcggcgtgggaaccccaagcc
ggggcagctccacctccccagcccgcgcccccggacgcctccgcctccg
cgcggcaggggcagatgcaaggcatcccggcgccctcccaggcgctcca
ggagccggcgccctggtctgcactcccctgcggcctgctgctggatgag
ctcctggcgagcccggagtttctgcagcaggcgcaacctctcctagaaa
cggaggccccgggggagctggaggcctcggaagaggccgcctcgctgga
agcacccctcagcgaggaagaataccgggctctgctggaggagctttag
gacgcggggttgggacggggtcgggtggttcggggcagggcggtggcct
ctctttcgcggggaacacctggctggctacggaggggcgtgtctccgcc
ccgccccctccaccgggctgaccggcctgggattcctgccttctaggtc
taggcccggtgagagactccacaccgcggagaactgccattctttcctg
ggcatcccggggatcccagagccggcccaggtaccagcagacctgcgcg
cagtgcgcaccccggctgacgtgcaagggagctcgctggcctctctgtg
cccttgttcttccgtgaaattctggctgaatgtctccccccaccttccg
acgctgtctaggcaaacctggattagagttacatctcctggatgattag
ttcagagatatattaaaatgccccctccctgtggatcctata.

In certain embodiments, the human DUX4 nucleic acid is the sequence set forth in GENBANK Accession No. NM_001293798.1 (variant 2):

(SEQ ID NO: 2)
atggccctcccgacaccctcggacagcaccctccccgcggaagcccggg
gacgaggacggcgacggagactcgtttggaccccgagccaaagcgaggc
cctgcgagcctgctttgagcggaacccgtacccgggcatcgccaccaga
gaacggctggcccaggccatcggcattccggagcccagggtccagattt
ggtttcagaatgagaggtcacgccagctgaggcagcaccggcgggaatc
tcggccctggcccgggagacgcggcccgccagaaggccggcgaaagcgg
accgccgtcaccggatcccagaccgccctgctcctccgagcctttgaga
aggatcgctttccaggcatcgccgcccgggaggagctggccagagagac
gggcctcccggagtccaggattcagatctggtttcagaatcgaagggcc
aggcacccgggacagggtggcagggcgcccgcgcaggcaggcggcctgt
gcagcgcggcccccggcgggggtcaccctgctccctcgtgggtcgcctt
cgcccacaccggcgcgtggggaacggggcttcccgcaccccacgtgccc
tgcgcgcctggggctctcccacagggggctttcgtgagccaggcagcga
gggccgcccccgcgctgcagcccagccaggccgcgccggcagaggggat
ctcccaacctgccccggcgcgcggggatttcgcctacgccgccccggct
cctccggacggggcgctctcccaccctcaggctcctcggtggcctccgc
acccgggcaaaagccgggaggaccgggacccgcagcgcgacggcctgcc
gggcccctgcgcggtggcacagcctgggcccgctcaagcggggccgcag
ggccaaggggtgcttgcgccacccacgtcccaggggagtccgtggtggg
gctggggccggggtccccaggtcgccggggcggcgtgggaaccccaagc
cggggcagctccacctccccagcccgcgcccccggacgcctccgcctcc
gcgcggcaggggcagatgcaaggcatcccggcgccctcccaggcgctcc
aggagccggcgccctggtctgcactcccctgcggcctgctgctggatga
gctcctggcgagcccggagtttctgcagcaggcgcaacctctcctagaa
acggaggccccgggggagctggaggcctcggaagaggccgcctcgctgg
aagcacccctcagcgaggaagaataccgggctctgctggaggagcttta
ggacgcggggtctaggcccggtgagagactccactccgcggagaactgc
ctttctttcctgggcatcccggggatcccagagccggcccaggtaccag
cagacctgcgcgcagtgcgcaccccggctgacgtgcaagggagctcgct
ggcctctctgtgcccttgttcttccgtgaaattctggctgaatgtctcc
ccccaccttccgacgctgtctaggcaaacctggattagagttacatctc
ctggatgattagttcagagatatattaaaatgccccctccctgtggatc
ctatag.

In certain embodiments, the DUX4 inhibitory compounds targeted the D4Z4 and adjacent sequences that encompass the DUX4 transcript as well as regions centromeric and telomeric to the DTJX4 transcript and comprises:

(SEQ ID NO: 3)
gggatgcgcgcgcctggggctctcccacagggggctttcgtgagccaggcagcgagggccgcccccgcgctgca
gcccagccaggccgcgacggcagagggggtctcccaacctgccccggcgcgcggggatttcgcctacgccgccccggctcctcc
ggacggggcgctctcccaccctcaggctcctcggtggcctccgcacccgggcaaaagccgggaggaccgggacccgcagcgcg
acggcctgccgggcccctgcgcggtggcacagcctgggcccgctcaagcggggccgcagggccaaggggtgcttgcgccaccc
acgtcccaggggagtccgtggtggggctggggccggggtccccaggtcgccggggcggcgtgggaaccccaagccggggcag
ctccacctccccagcccgcgcccccggacgcctccgcgcggcaggggcagatgcaaggcatcccggcgccctcccaggcgctcc
aggagccggcgccctggtctgcactcccctgcggcctgctgctggatgagctcctggcgagcccggagtttctgcagcaggcgcaa
cctctcctagaaacggaggccccgggggagctggaggcctcggaagaggccgcctcgctggaagcacccctcagcgaggaaga
ataccgggctctgctggaggagctttaggacgcggggttgggacggggtcgggtggttcggggcagggcggtggcctctctttcgc
ggggaacgcctggctggctacggaggggcgtgtctccgccccgccccctccaccgggctgaccggcctgggattcctgccttctag
gtctaggcccggtgagagactccacaccgcggagaactgccattctttcctgggcatcccggggatcccagagccggcccaggtac
cagcaggtgggccgcctactgcgcacgcgcgggtttgcgggcagccgcctgggctgtgggagcagcccgggcagagctctcctg
cctctccaccagcccaccccgccgcctgaccgccccctccccacccccaccccccacccccggaaaacgcgtcgtcccctgggct
gggtggagacccccgtcccgcgaaacaccgggccccgcgcagcgtccgggcctgacaccgctccggcggctcgcctcctctgcg
cccccgcgccaccgtcgcccgcccgcccgggcccctgcagccgcccaggtgccagcacggagcgcctggcggcggaacgcag
accccaggcccggcgcacaccggggacgctgagcgttccaggcgggagggaaggcgggcagagatggagagaggaacggga
gacctagaggggcggaaggacgggcggagggacgttaggagggagggagggaggcagggaggcagggaggaacggaggg
aaagacagagcgacgcagggactgggggcgggcgggagggagccggggacggacggggggaggaaggcagggaggaaaa
gcggtcctcggcctccgggagtagcgggacccccgccctccgggaaaacggtcagcgtccggcgcgggctgagggctgggccc
acagccgccgcgccggccggcgcggcacccattcgccccggttccgtggcccagggagtgggcggtttcctccgggacaaaaga
ccgggactcgggttgccgtcgggttttcacccgcgcggttcacagaccgcacatccccaggctgagccctgcaacgcggcgcgag
gccgacagccccggccacggaggagccacacgcaggacgacggaggcgtgattttggtttccgcgtggctttgccctccgcaagg
cggcctgttgctcacgtctctccggcccccgaaaggctggccatgccgactgtttgctcccggagctctgcgggcacccggaaacat
gcagggaagggtgcaagcccggcatggtgccttcgctctccttgccaggttccaaaccggccacactgcagactccccacgttgccg
cacgcgggaatccatcgtcaggccatcacgccggggaggcatctcctctctggggtctcgctctggtcttctacgtggaaatgaacga
gagccacacgcctgcgtgtgcgagaccgtcccggcaacggcgacgcccacaggcattgcctccttcacggagagagggcctggc
acactcaagactcccacggaggttcagttccacactcccctccaccctcccaggctggtttctccctgctgccgacgcgtgggagccc
agagagcggcttcccgttcccgcgggatccctggagaggtccggagagccggcccccgaaacgcgcccccctcccccctcccccc
tctccccgttcctcttcgtctctccggccccaccaccaccaccgccaccacgccctcccccaccaccccccccccccccaccaccacc
accaccaccccgccggccggccccaggcctcgacgccctgggtcccttccggggtggggcgggctgtcccaggggggctcaccg
ccattcatgaaggggtggagcctgcctgcctgtgggcctttacaagggcggctggctgggtggctggctgtccgggcaggccccctg
gctgcacctgccgcagtgcacagtccggctgaggtgcacgggagcccgccggcctctctctgcccgcgtccgtccgtgaaattccg
gccggggctcaccgcgatggccctcccgacaccctcggacagcaccctccccgcggaagcccggggacgaggacggcgacgg
agactcgtttggaccccgagccaaagcgaggccctgcgagcctgctttgagcggaacccgtacccgggcatcgccaccagagaac
ggctggcccaggccatcggcattccggagcccagggtccagatttggtttcagaatgagaggtcacgccagctgaggcagcaccgg
cgggaatctcggccctggcccgggagacgcggcccgccagaaggccggcgaaagcggaccgccgtcaccggatcccagaccg
ccctgctcctccgagcctttgagaaggatcgctttccaggcatcgccgcccgggaggagctggccagagagacgggcctcccgga
gtccaggattcagatctggtttcagaatcgaagggccaggcacccgggacagggtggcagggcgcccgcgcaggcaggcggcct
gtgcagcgcggcccccggcgggggtcaccctgctccctcgtgggtcgccttcgcccacaccggcgcgtggggaacggggcttccc
gcaccccacgtgccctgcgcgcctggggctctcccacagggggctttcgtgagccaggcagcgagggccgcccccgcgctgcag
cccagccaggccgcgccggcagaggggatctcccaacctgccccggcgcgcggggatttggcctacgccgccccggctcctccg
gacggggcgctctcccaccctcaggctcctcggtggcctccgcacccgggcaaaagccgggaggaccgggacccgcagcgcga
cggcctgccgggcccctgcgcggtggcacagcctgggcccgctcaagcggggccgcagggccaaggggtgcttgcgccaccca
cgtcccaggggagtccgtggtggggctggggccggggtccccaggtcgccggggcggcgtgggaaccccaagccggggcagct
ccacctccccagcccgcgcccccggacgcctccgcctccgcgcggcaggggcagatgcaaggcatcccggcgccctcccaggc
gctccaggagccggcgccctggtctgcactcccctgcggcctgctgctggatgagctcctggcgagcccggagtttctgcagcagg
cgcaacctctcctagaaacggaggccccgggggagctggaggcctcggaagaggccgcctcgctggaagcacccctcagcgag
gaagaataccgggctctgctggaggagctttaggacgcggggttgggacggggtcgggtggttcggggcagggcggtggcctctc
tttcgcggggaacacctggctggctacggaggggcgtgtctccgccccgccccctccaccgggctgaccggcctgggattcctgcct
tctaggtctaggcccggtgagagactccacaccgcggagaactgccattattcctgggcatcccggggatcccagagccggcccag
gtaccagcaggtgggccgcctactgcgcacgcgcgggtagcgggcagccgcctgggctgtgggagcagcccgggcagagctct
cctgcctctccaccagcccaccccgccgcctgaccgccccctccccacccccaccccccacccccggaaaacgcgtcgtcccctgg
gctgggtggagacccccgtcccgcgaaacaccgggccccgcgcagcgtccgggcctgacaccgctccggcggctcgcctcctct
gcgcccccgcgccaccgtcgcccgcccgcccgggcccctgcagccgcccaggtgccagcacggagcgcctggcggcggaacg
cagaccccaggcccggcgcacaccggggacgctgagcgttccaggcgggagggaaggcgggcagagatggagagaggaacg
ggagacctagaggggcggaaggacgggcggagggacgttaggagggagggagggaggcagggaggcagggaggaacgga
gggaaagacagagcgacgcagggactgggggcgggcgggagggagccgggggacggggggaggaaggcagggaggaaaa
gcggtcctcggcctccgggagtagcgggacccccgccctccgggaaaacggtcagcgtccggcgcgggctgagggctgggccc
acagccgccgcgccggccggcggggcaccacccattcgccccggttccggggcccagggagtgggcggtttcctccgggacaaa
agaccgggactcgggttgccgtcgggtcttcacccgcgcggttcacagaccgcacatccccaggctcagccctgcaacgcggcgc
gaggccgacagccccggccacggaggagccacacgcaggacgacggaggcgtgattttggtttccgcgtggctttgccctccgca
aggcggcctgttgctcacgtctctccggcccccgaaaggctggccatgccgactgtttgctcccggagctctgcgggcacccggaaa
catgcagggaagggtgcaagcccggcacggtgccttcgctctccttgccaggttccaaaccggccacactgcagactccccacgttg
ccgcacgcgggaatccatcgtcaggccatcacgccggggaggcatctcctctctggggtctcgctctggtcttctacgtggaaatgaa
cgagagccacacgcctgcgtgtgcgagaccgtcccggcaacggcgacgcccacaggcattgcctccttcacggagagagggcct
ggcacactcaagactcccacggaggttcagttccacactcccctccaccctcccaggctggtttctccctgctgccgacgcgtgggag
cccagagagcggcttcccgttcccgcgggatccctggagaggtccggagagccggcccccgaaacgcgcccccctcccccctccc
ccctctcccccttcctcttcgtctctccggccccaccaccaccaccgccaccacgccctccccccccaccccccccccccaccaccac
caccaccaccaccccgccggccggccccaggcctcgacgccctgggtcccttccggggtggggcgggctgtcccaggggggctc
accgccattcatgaaggggtggagcctgcctgcctgtgggcctttacaagggcggctggctggctggctggctgtccgggcaggcct
cctggctgcacctgccgcagtgcacagtccggctgaggtgcacgggagcccgccggcctctctctgcccgcgtccgtccgtgaaatt
ccggccggggctcaccgcgatggccctcccgacaccctcggacagcaccctccccgcggaagcccggggacgaggacggcgac
ggagactcgtttggaccccgagccaaagcgaggccctgcgagcctgctttgagcggaacccgtacccgggcatcgccaccagaga
acggctggcccaggccatcggcattccggagcccagggtccagatttggtttcagaatgagaggtcacgccagctgaggcagcacc
ggcgggaatctcggccctggcccgggagacgcggcccgccagaaggccggcgaaagcggaccgccgtcaccggatcccagac
cgccctgctcctccgagcctttgagaaggatcgctttccaggcatcgccgcccgggaggagctggccagagagacgggcctcccg
gagtccaggattcagatctggtttcagaatcgaagggccaggcacccgggacagggtggcagggcgcccgcgcaggcaggcggc
ctgtgcagcgcggcccccggcgggggtcaccctgctccctcgtgggtcgccttcgcccacaccggcgcgtggggaacggggcttc
ccgcaccccacgtgccctgcgcgcctggggctctcccacagggggctttcgtgagccaggcagcgagggccgcccccgcgctgc
agcccagccaggccgcgccggcagaggggatctcccaacctgccccggcgcgcggggatttcgcctacgccgccccggctcctc
cggacggggcgctctcccaccctcaggctcctcgctggcctccgcacccgggcaaaagccgggaggaccgggacccgcagcgc
gacggcctgccgggcccctgcgcggtggcacagcctgggcccgctcaagcggggccgcagggccaaggggtgcttgcgccacc
cacgtcccaggggagtccgtggtggggctggggccggggtccccaggtcgccggggcggcgtgggaaccccaagccggggca
gctccacctccccagcccgcgcccccggacgcctccgcctccgcgcggcaggggcagatgcaaggcatcccggcgccctcccag
gcgctccaggagccggcgccctggtctgcactcccctgcggcctgctgctggatgagctcctggcgagcccggagtttctgcagca
ggcgcaacctctcctagaaacggaggccccgggggagctggaggcctcggaagaggccgcctcgctggaagcacccctcagcg
aggaagaataccgggctctgctggaggagctttaggacgcggggttgggacggggtcgggtggttcggggcagggccgtggcctc
tctttcgcggggaacacctggctggctacggaggggcgtgtctccgccccgccccctccaccgggctgaccggcctgggattcctgc
cttctaggtctaggcccggtgagagactccacaccgcggagaactgccattctacctgggcatcccggggatcccagagccggccc
aggtaccagcaggtgggccgcctactgcgcacgcgcgggtttgcgggcagccgcctgggctgtgggagcagcccgggcagagct
ctcctgcctctccaccagcccaccccgccgcctgaccgccccctccccacccccaccccccacccccggaaaacgcgtcgtcccct
gggctgggtggagacccccgtcccgcgaaacaccgggccccgcgcagcgtccgggcctgacaccgctccggcggctcgcctcct
ctgcgcccccgcgccaccgtcgcccgcccgcccgggcccctgcagcctcccagctgccagcgcggagctcctggcggtcaaaag
catacctctgtctgtctttgcccgcttcctggctagacctgcgcgcagtgcgcaccccggctgacgtgcaagggagctcgctggcctct
ctgtgcccttgttcttccgtgaaattctggctgaatgtctccccccaccttccgacgctgtctaggcaaacctggattagagttacatctcct
ggatgattagttcagagatatattaaaatgccccctccctgtggatcctatagaagatttgcatcttttgtgtgatgagtgcagagatatgtc
acaatatcccctgtagaaaaagcctgaaattggtttacataacttcggtgatcagtgcagatgtgtttcagaactccatagtagactgaac
ctagagaatggttacatcacttaggtgatcagtgtagagatatgttaaaattctcgtgtagacagagcctagacaattgttacatcacctag
tgatcagtgcagggataagtcataaagcctcctgtaggcagagtgtaggcaagtgttccctccctgggctgatcagtgcagagatatct
cacaaagcccctataagccaaaccttgacaagggttacatcacctgtagagcagtggaaatatatatcacaaagccccctgtagacaa
agcccagacaatttttacatctcctgagtgagcattggagagatctgtcacaatgcccctgtaggcagagcttagacaagtgttacatca
cctgggtgatcagtgcagagatatgtcaaaacgctcctgtagtctgaacctagacaggagttacatcaccttggggatcagtgcagaga
tacgtgagaattcc.

In some embodiments, the nucleic acid inhibitor comprises one that targets the following sequence:

(SEQ ID NO: 71)
GGTACCAGCAGGTGGGCCGCCTACTGCGCACGCGCGGGTTTGCGGGCAG
CCGCCTGGGCTGTGGGAGCAGCCCGGGCAGAGCTCTCCTGCCTCTCCAC
CAGCCCACCCCGCCGCCTGACCGCCCCCTCCCCACCCCCACCCCCCACC
CCCGGAAAACGCGTCGTCCCCTGGGCTGGGTGGAGACCCCCGTCCCGCG
AAACACCGGGCCCCGCGCAGCGTCCGGGCCTGACACCGCTCCGGCGGCT
CGCCTCCTCTGCGCCCCCGCGCCACCGTCGCCCGCCCGCCCGGGCCCCT
GCAGCCGCCCAGGTGCCAGCACGGAGCGCCTGGCGGCGGAACGCAGACC
CCAGGCCCGGCGCACACCGGGGACGCTGAGCGTTCCAGGCGGGAGGGAA
GGCGGGCAGAGATGGAGAGAGGAACGGGAGACCTAGAGGGGCGGAAGGA
CGGGCGGAGGGACGTTAGGAGGGAGGGAGGGAGGCAGGGAGGCAGGGAG
GAACGGAGGGAAAGACAGAGCGACGCAGGGACTGGGGGCGGGCGGGAGG
GAGCCGGGGACGGACGGGGGGAGGAAGGCAGGGAGGAAAAGCGGTCCTC
GGCCTCCGGGAGTAGCGGGACCCCCGCCCTCCGGGAAAACGGTCAGCGT
CCGGCGCGGGCTGAGGGCTGGGCCCACAGCCGCCGCGCCGGCCGGCGCG
GCACCCATTCGCCCCGGTTCCGTGGCCCAGGGAGTGGGCGGTTTCCTCC
GGGACAAAAGACCGGGACTCGGGTTGCCGTCGGGTTTTCACCCGCGCGG
TTCACAGACCGCACATCCCCAGGCTGAGCCCTGCAACGCGGCGCGAGGC
CGACAGCCCCGGCCACGGAGGAGCCACACGCAGGACGACGGAGGCGTGA
TTTTGGTTTCCGCGTGGCTTTGCCCTCCGCAAGGCGGCCTGTTGCTCAC
GTCTCTCCGGCCCCCGAAAGGCTGGCCATGCCGACTGTTTGCTCCCGGA
GCTCTGCGGGCACCCGGAAACATGCAGGGAAGGGTGCAAGCCCGGCATG
GTGCCTTCGCTCTCCTTGCCAGGTTCCAAACCGGCCACACTGCAGACTC
CCCACGTTGCCGCACGCGGGAATCCATCGTCAGGCCATCACGCCGGGGA
GGCATCTCCTCTCTGGGGTCTCGCTCTGGTCTTCTACGTGGAAATGAAC
GAGAGCCACACGCCTGCGTGTGCGAGACCGTCCCGGCAACGGCGACGCC
CACAGGCATTGCCTCCTTCACGGAGAGAGGGCCTGGCACACTCAAGACT
CCCACGGAGGTTCAGTTCCACACTCCCCTCCACCCTCCCAGGCTGGTTT
CTCCCTGCTGCCGACGCGTGGGAGCCCAGAGAGCGGCTTCCCGTTCCCG
CGGGATCCCTGGAGAGGTCCGGAGAGCCGGCCCCCGAAACGCGCCCCCC
TCCCCCCTCCCCCCTCTCCCCGTTCCTCTTCGTCTCTCCGGCCCCACCA
CCACCACCGCCACCACGCCCTCCCCCACCACCCCCCCCCCCCCCACCAC
CACCACCACCACCCCGCCGGCCGGCCCCAGGCCTCGACGCCCTGGGTCC
CTTCCGGGGTGGGGCGGGCTGTCCCAGGGGGGCTCACCGCCATTCATGA
AGGGGTGGAGCCTGCCTGCCTGTGGGCCTTTACAAGGGCGGCTGGCTGG
GTGGCTGGCTGTCCGGGCAGGCCCCCTGGCTGCACCTGCCGCAGTGCAC
AGTCCGGCTGAGGTGCACGGGAGCCCGCCGGCCTCTCTCTGCCCGCGTC
CGTCCGTGAAATTCCGGCCGGGGCTCACCGCGATGGCCCTCCCGACACC
CTCGGACAGCACCCTCCCCGCGGAAGCCCGGGGACGAGGACGGCGACGG
AGACTCGTTTGGACCCCGAGCCAAAGCGAGGCCCTGCGAGCCTGCTTTG
AGCGGAACCCGTACCCGGGCATCGCCACCAGAGAACGGCTGGCCCAGGC
CATCGGCATTCCGGAGCCCAGGGTCCAGATTTGGTTTCAGAATGAGAGG
TCACGCCAGCTGAGGCAGCACCGGCGGGAATCTCGGCCCTGGCCCGGGA
GACGCGGCCCGCCAGAAGGCCGGCGAAAGCGGACCGCCGTCACCGGATC
CCAGACCGCCCTGCTCCTCCGAGCCTTTGAGAAGGATCGCTTTCCAGGC
ATCGCCGCCCGGGAGGAGCTGGCCAGAGAGACGGGCCTCCCGGAGTCCA
GGATTCAGATCTGGTTTCAGAATCGAAGGGCCAGGCACCCGGGACAGGG
TGGCAGGGCGCCCGCGCAGGCAGGCGGCCTGTGCAGCGCGGCCCCCGGC
GGGGGTCACCCTGCTCCCTCGTGGGTCGCCTTCGCCCACACCGGCGCGT
GGGGAACGGGGCTTCCCGCACCCCACGTGCCCTGCGCGCCTGGGGCTCT
CCCACAGGGGGCTTTCGTGAGCCAGGCAGCGAGGGCCGCCCCCGCGCTG
CAGCCCAGCCAGGCCGCGCCGGCAGAGGGGATCTCCCAACCTGCCCCGG
CGCGCGGGGATTTGGCCTACGCCGCCCCGGCTCCTCCGGACGGGGCGCT
CTCCCACCCTCAGGCTCCTCGGTGGCCTCCGCACCCGGGCAAAAGCCGG
GAGGACCGGGACCCGCAGCGCGACGGCCTGCCGGGCCCCTGCGCGGTGG
CACAGCCTGGGCCCGCTCAAGCGGGGCCGCAGGGCCAAGGGGTGCTTGC
GCCACCCACGTCCCAGGGGAGTCCGTGGTGGGGCTGGGGCCGGGGTCCC
CAGGTCGCCGGGGCGGCGTGGGAACCCCAAGCCGGGGCAGCTCCACCTC
CCCAGCCCGCGCCCCCGGACGCCTCCGCCTCCGCGCGGCAGGGGCAGAT
GCAAGGCATCCCGGCGCCCTCCCAGGCGCTCCAGGAGCCGGCGCCCTGG
TCTGCACTCCCCTGCGGCCTGCTGCTGGATGAGCTCCTGGCGAGCCCGG
AGTTTCTGCAGCAGGCGCAACCTCTCCTAGAAACGGAGGCCCCGGGGGA
GCTGGAGGCCTCGGAAGAGGCCGCCTCGCTGGAAGCACCCCTCAGCGAG
GAAGAATACCGGGCTCTGCTGGAGGAGCTTTAGGACGCGGGGTTGGGAC
GGGGTCGGGTGGTTCGGGGCAGGGCGGTGGCCTCTCTTTCGCGGGGAAC
ACCTGGCTGGCTACGGAGGGGCGTGTCTCCGCCCCGCCCCCTCCACCGG
GCTGACCGGCCTGGGATTCCTGCCTTCTAGGTCTAGGCCCGGTGAGAGA
CTCCACACCGCGGAGAACTGCCATTCTTTCCTGGGCATCCCGGGGATCC
CAGAGCCGGCCCAGGTACCAGCAGGTGGGCCGCCTACTGCGCACGCGCG
GGTTTGCGGGCAGCCGCCTGGGCTGTGGGAGCAGCCCGGGCAGAGCTCT
CCTGCCTCTCCACCAGCCCACCCCGCCGCCTGACCGCCCCCTCCCCACC
CCCCACCCCCCACCCCCGGAAAACGCGTCGTCCCCTGGGCTGGGTGGAG
ACCCCCGTCCCGCGAAACACCGGGCCCCGCGCAGCGTCCGGGCCTGACA
CCGCTCCGGCGGCTCGCCTCCTCTGCGCCCCCGCGCCACCGTCGCCCGC
CCGCCCGGGCCCCTGCAGCCGCCCAGGTGCCAGCACGGAGCGCCTGGCG
GCGGAACGCAGACCCCAGGCCCGGCGCACACCGGGGACGCTGAGCGTTC
CAGGCGGGAGGGAAGGCGGGCAGAGATGGAGAGAGGAACGGGAGACCTA
GAGGGGCGGAAGGACGGGCGGAGGGACGTTAGGAGGGAGGGAGGGAGGC
AGGGAGGCAGGGAGGAACGGAGGGAAAGACAGAGCGACGCAGGGACTGG
GGGCGGGCGGGAGGGAGCCGGGGGACGGGGGGAGGAAGGCAGGGAGGAA
AAGCGGTCCTCGGCCTCCGGGAGTAGCGGGACCCCCGCCCTCCGGGAAA
ACGGTCAGCGTCCGGCGCGGGCTGAGGGCTGGGCCCACAGCCGCCGCGC
CGGCCGGCGGGGCACCACCCATTCGCCCCGGTTCCGGGGCCCAGGGAGT
GGGCGGTTTCCTCCGGGACAAAAGACCGGGACTCGGGTTGCCGTCGGGT
CTTCACCCGCGCGGTTCACAGACCGCACATCCCCAGGCTCAGCCCTGCA
ACGCGGCGCGAGGCCGACAGCCCCGGCCACGGAGGAGCCACACGCAGGA
CGACGGAGGCGTGATTTTGGTTTCCGCGTGGCTTTGCCCTCCGCAAGGC
GGCCTGTTGCTCACGTCTCTCCGGCCCCCGAAAGGCTGGCCATGCCGAC
TGTTTGCTCCCGGAGCTCTGCGGGCACCCGGAAACATGCAGGGAAGGGT
GCAAGCCCGGCACGGTGCCTTCGCTCTCCTTGCCAG*GTTCCAAACCGG
CCACACTGCAGACTCCCCACGTTGCCGCACGCGGGAATCCATCGTCAGG
CCATCACGCCGGGGAGGCATCTCCTCTCTGGGGTCTCGCTCTGGTCTTC
TACGTGGAAATGAACGAGAGCCACACGCCTGCGTGTGCGAGACCGTCCC
GGCAACGGCGACGCCCACAGGCATTGCCTCCTTCACGGAGAGAGGGCCT
GGCACACTCAAGACTCCCACGGAGGTTCAGTTCCACACTCCCCTCCACC
CTCCCAGGCTGGTTTCTCCCTGCTGCCGACGCGTGGGAGCCCAGAGAGC
GGCTTCCCGTTCCCGCGGGATCCCTGGAGAGGTCCGGAGAGCCGGCCCC
CGAAACGCGCCCCCCTCCCCCCTCCCCCCTCTCCCCCTTCCTCTTCGTC
TCTCCGGCCCCACCACCACCACCGCCACCACGCCCTCCCCCCCCACCCC
CCCCCCCCACCACCACCACCACCACCACCCCGCCGGCCGGCCCCAGGCC
TCGACGCCCTGGGTCCCTTCCGGGGTGGGGCGGGCTGTCCCAGGGGGGC
TCACCGCCATTCATGAAGGGGTGGAGCCTGCCTGCCTGTGGGCCTTTAC
AAGGGCGGCTGGCTGGCTGGCTGGCTGTCCGGGCAGGCCTCCTGGCTGC
ACCTGCCGCAGTGCACAGTCCGGCTGAGGTGCACGGGAGCCCGCCGGCC
TCTCTCTGCCCGCGTCCGTCCGTGAAATTCCGGCCGGGGCTCACCGCGA
TGGCCCTCCCGACACCCTCGGACAGCACCCTCCCCGCGGAAGCCCGGGG
ACGAGGACGGCGACGGAGACTCGTTTGGACCCCGAGCCAAAGCGAGGCC
CTGCGAGCCTGCTTTGAGCGGAACCCGTACCCGGGCATCGCCACCAGAG
AACGGCTGGCCCAGGCCATCGGCATTCCGGAGCCCAGGGTCCAGATTTG
GTTTCAGAATGAGAGGTCACGCCAGCTGAGGCAGCACCGGCGGGAATCT
CGGCCCTGGCCCGGGAGACGCGGCCCGCCAGAAGGCCGGCGAAAGCGGA
CCGCCGTCACCGGATCCCAGACCGCCCTGCTCCTCCGAGCCTTTGAGAA
GGATCGCTTTCCAGGCATCGCCGCCCGGGAGGAGCTGGCCAGAGAGACG
GGCCTCCCGGAGTCCAGGATTCAGATCTGGTTTCAGAATCGAAGGGCCA
GGCACCCGGGACAGGGTGGCAGGGCGCCCGCGCAGGCAGGCGGCCTGTG
CAGCGCGGCCCCCGGCGGGGGTCACCCTGCTCCCTCGTGGGTCGCCTTC
GCCCACACCGGCGCGTGGGGAACGGGGCTTCCCGCACCCCACGTGCCCT
GCGCGCCTGGGGCTCTCCCACAGGGGGCTTTCGTGAGCCAGGCAGCGAG
GGCCGCCCCCGCGCTGCAGCCCAGCCAGGCCGCGCCGGCAGAGGGGATC
TCCCAACCTGCCCCGGCGCGCGGGGATTTCGCCTACGCCGCCCCGGCTC
CTCCGGACGGGGCGCTCTCCCACCCTCAGGCTCCTCGCTGGCCTCCGCA
CCCGGGCAAAAGCCGGGAGGACCGGGACCCGCAGCGCGACGGCCTGCCG
GGCCCCTGCGCGGTGGCACAGCCTGGGCCCGCTCAAGCGGGGCCGCAGG
GCCAAGGGGTGCTTGCGCCACCCACGTCCCAGGGGAGTCCGTGGTGGGG
CTGGGGCCGGGGTCCCCAGGTCGCCGGGGCGGCGTGGGAACCCCAAGCC
GGGGCAGCTCCACCTCCCCAGCCCGCGCCCCCGGACGCCTCCGCCTCCG
CGCGGCAGGGGCAGATGCAAGGCATCCCGGCGCCCTCCCAGGCGCTCCA
GGAGCCGGCGCCCTGGTCTGCACTCCCCTGCGGCCTGCTGCTGGATGAG
CTCCTGGCGAGCCCGGAGTTTCTGCAGCAGGCGCAACCTCTCCTAGAAA
CGGAGGCCCCGGGGGAGCTGGAGGCCTCGGAAGAGGCCGCCTCGCTGGA
AGCACCCCTCAGCGAGGAAGAATACCGGGCTCTGCTGGAGGAGCTTTAG
GACGCGGGGTTGGGACGGGGTCGGGTGGTTCGGGGCAGGGCCGTGGCCT
CTCTTTCGCGGGGAACACCTGGCTGGCTACGGAGGGGCGTGTCTCCGCC
CCGCCCCCTCCACCGGGCTGACCGGCCTGGGATTCCTGCCTTCTAGGTC
TAGGCCCGGTGAGAGACTCCACACCGCGGAGAACTGCCATTCTTTCCTG
GGCATCCCGGGGATCCCAGAGCCGGCCCAGGTACCAGCAGGTGGGCCGC
CTACTGCGCACGCGCGGGTTTGCGGGCAGCCGCCTGGGCTGTGGGAGCA
GCCCGGGCAGAGCTCTCCTGCCTCTCCACCAGCCCACCCCGCCGCCTGA
CCGCCCCCTCCCCACCCCCACCCCCCACCCCCGGAAAACGCGTCGTCCC
CTGGGCTGGGTGGAGACCCCCGTCCCGCGAAACACCGGGCCCCGCGCAG
CGTCCGGGCCTGACACCGCTCCGGCGGCTCGCCTCCTCTGCGCCCCCGC
GCCACCGTCGCCCGCCCGCCCGGGCCCCTGCAGCCTCCCAGCTGCCAGC
ACGGAGCGCCTGGCGGTCAAAAGCATACCTCTGTCTGTCTTTGCCCGCT
TCCTGGCTAGACCTGCGCGCAGTGCGCACCCCGGCTGACGTGCAAGGGA
GCTCGCTGGCCTCTCTGTGCCCTTGTTCTTCCGTGAAATTCTGGCTGAA
TGTCTCCCCCCACCTTCCGACGCTGTCTAGGCAAACCTGGATTAGAGTT
ACATCTCCTGGATGATTAGTTCAGAGATATATTAAAATGCCCCCTCCCT
GTGGATCCTATAGAAGATTTGCATCTTTTGTGTGATGAGTGCAGAGATA
TGTCACAATATCCCCTGTAGAAAAAGCCTGAAATTGATTTACAGAACTT
CGGTGATCAGTGCAGATGTGTTTCAGAACTCCATAGTAGACTGAACCTA
GAGAATGGTTACATCACTTAGGTGATCAGTGTAGAGATATGTTAAAATT
CTCGTGTAGACAGAGCCTAGACAATTGTTACATCACCTAGTGATCAGTG
CAGGGATAAGTCATAAAGCCTCCTGTAGGCAGAGTGTAGGCAAGTGTTC
CCTCCCTGGGCTGATCAGTGCAGAGATATCTCACAAAGCCCCTATAAGC
CAAACCTTGACAAGGGTTACATCACCTGTTTGAGCAGTGGAAATATATA
TCACAAAGCCCCCTGTAGACAAAGCCCAGACAATTTTTACATCTCCTGA
GTGAGCATTGGAGAGATCTGTCACAATGCCCCTGTAGGCAGAGCTTAGA
CAAGTGTTACATCACCTGGGTGATCAGTGCAGAGATGTGTCAAAACGCT
CCTGTAGTCTGAACCTAGACAGGAGTTACATCACCTTGGGGATCAGTGC
AGAGATACGTGAGAATTCC.

The nucleic acid inhibitor may be a nucleic acid that comprises or is complementary to all or a portion of SEQ ID NO:1, 2, 3, or 71. In some embodiments, the nucleic acid inhibitor is complementary to or comprises at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, 90, 95, 100, 110, 120, 130, 140, 150, or 200 contiguous nucleotides (or any derivable range therein) from SEQ ID NO:1, 2 or 3. In some embodiments, the nucleic acid inhibitor may be a nucleic acid that is at least, at most, or exactly 75, 80, 85, 86, 87, 88, 89, 90, 91, 92, 93, 94, 95, 96, 97, 98, 99, or 100% identical (or any derivable range therein) to a nucleic acid complementary to or comprises at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 65, 70, 75, 80, 85, 90, 95, 100, 110, 120, 130, 140, 150, or 200 contiguous nucleotides (or any derivable range therein) from SEQ ID NO:1, 2, 3, or 71.

Exemplary nucleic acid inhibitors are known in the art, commercially available, and some are described herein. For example, siRNAs suitable as DUX4 inhibitors are sold commercially by Dharmacon, Life Technologies, and Qiagen.

A nucleic acid inhibitor may also target a non-coding region DUX4. In some embodiments, the nucleic acid inhibitor is a nucleic acid decoy, such as those described in US20180016577, which is herein incorporated by reference. Exemplary nucleic acid decoys include double-stranded DNA comprising one of the following sequences:

(SEQ ID NO: 5)
gtaatccaatcat,
(SEQ ID NO: 6)
gaggtaatccaatcatgga,
(SEQ ID NO: 7)
cgtaatccaatcagc,
(SEQ ID NO: 8)
tgcgtaatccaatcagcgt,
(SEQ ID NO: 9)
cctgtgggaggtaatccaatcatggaggcagcctgtgggaggtaatcca
atcatggaggcaga,
(SEQ ID NO: 10)
gcguacgauacctgtgggaggtaatccaatcatggaggcagcctgtggg
aggtaatccaatcatggaggcagaaucccaugc,
(SEQ ID NO: 11)
gtgggaggtaatccaatcatggaggcag,
(SEQ ID NO: 12)
cccatgcgtaatccaatcagcgtacgat,
(SEQ ID NO: 13)
cctgtgggaggtaatccaatcatggaggcagcct,
and
(SEQ ID NO: 14)
gaccctgtgggaggtaatccaatcatggaggcagtttccc.

Sequence elements to be targeted by (i.e., selected to be bound by) antisense agents may be chosen from the group comprising or consisting of splice donor sites (i.e., 5′ splice sites), splice acceptor sites (i.e., 3′ splice sites), pyrimidine-rich or polypyrimidine tracts upstream of (i.e., 5′ relative to) splice acceptor sites, exon-intron boundaries, intron-exon boundaries, branch sites, exonic splicing enhancer elements of the DUX4 genes, polyadenylation sequences, and D4Z4 (SEQ ID NO:71) regions. Splice donor sites and splice acceptor sites, exon-intron boundaries and intron-exon boundaries may be readily accessible for targeting and may thus constitute preferred sequence elements as intended herein.

Antisense agents as disclosed herein may bind to a whole sequence element required for splicing DUX4 (i.e., may wholly overlap with or wholly anneal to such sequence element). Alternatively, antisense agents as disclosed herein may bind to one or more portions of a sequence element required for splicing DUX4 (e.g., may partly overlap with or partly anneal to such sequence element).

Reference to “binding to a sequence element required for splicing” also encompasses antisense agents that bind at a position sufficiently close to said element. For example, the antisense agents may bind at a position sufficiently close to said element to disrupt the binding and function of splicing machinery that would normally mediate a particular splicing reaction occurring at that element (e.g., such agents may bind to pre-mRNA at a position within about 3, about 6, or about 9 bases of said element).

Antisense agents as intended herein preferably comprise or denote antisense molecules such as antisense nucleic acid molecules or antisense nucleic acid analogue molecules. Antisense agents may refer to antisense oligonucleotides or antisense oligonucleotide analogues. By means of an example and not limitation, such antisense agents or molecules may be between about 10 and about 100 nucleotides or nucleotide analogues in length, between about 12 and about 80 nucleotides or nucleotide analogues in length, between about 15 and about 50 nucleotides or nucleotide analogues in length, or between about 20 and about 40 (such as, e.g., between about 20 and about 30) nucleotides or nucleotide analogues in length.

Also disclosed herein are antisense agents including antisense nucleic acid analogue molecules, such as, e.g., antisense oligonucleotide analogues. Exemplary antisense oligonucleotide analogues comprise a 2′-O-methylated phosphorothioate backbone or a phosphorodiamidate morpholino backbone.

Further by means of an example and not limitation, such antisense agents or molecules may be configured to bind to (anneal with) a sequence region, more particularly a region in DUX4 or DUX4c (pre-mRNA) sequence, wherein said region is at least about 10 nucleotides in length, preferably at least about 12 nucleotides in length, also preferably at least about 15 nucleotides in length, more preferably at least about 20 nucleotides in length, even more preferably at least about 25 or at least about 30 nucleotides in length, such as for example between about 10 and about 100 nucleotides in length, preferably between about 12 and about 80 nucleotides in length, also preferably between about 15 and about 50 nucleotides in length, and more preferably between about 20 and about 40 (such as, e.g., between about 20 and about 30) nucleotides in length, wherein the reference to nucleotides may preferably denote consecutive nucleotides.

Exemplary antisense nucleic acids include:

(SEQ ID NO: 15)
CUCUCACCGGGCCUAGACCUAGAAG;
(SEQ ID NO: 16)
UGCGCACUGCGCGCAGGUCUAGCCA;
(SEQ ID NO: 17)
ACUGCGCGCAGGUCUAGCCAGGAAG;
(SEQ ID NO: 18)
CGGGGUGCGCACUGCGCGCAGGUCU;
(SEQ ID NO: 19)
UGCGCACUGCGCGCAGGUCUAGCCAGGAAG;
(SEQ ID NO: 20)
ACUGCGCGCAGGUCUAGCCAGGAAGCGGGC;
(SEQ ID NO: 21)
ACCCGACCCCGUCCCAACCCCGCGU;
and
(SEQ ID NO: 22)
GGGCAUUUUAAUAUAUCUCUGAACU.

In some embodiments, the DUX4 nucleic acid inhibitor comprises a double-stranded siRNA, wherein the antisense strand of a the double-stranded siRNA comprises a nucleic acid sequence comprising at least, at most, or exactly 10, 11, 12, 13, 14, 15, 16, 17, 18, 19, 20, 21, 22, 23, 24, or 25 (or any derivable range therein) contiguous nucleobases of any of the nucleobase sequences of ttcctctctccatctctgc (SEQ ID NO:23), ttgtcccggaggaaaccgc (SEQ ID NO:24), ttccctgcatgtttccgggtgcccg (SEQ ID NO:25), cttccctgcatgtttccgg (SEQ ID NO:26), ctccgtgggagtcttgagtgtgcca (SEQ ID NO:27), caccccttcatgaatggcg (SEQ ID NO:28), caggctccaccccttcatg (SEQ ID NO:29), ttccgctcaaagcaggctc (SEQ ID NO:30), aaagcgatccttctcaaaggctcgg (SEQ ID NO:31), cctgcgcgggcgccctgcc (SEQ ID NO:32), tatctctgaactaatcatc, or atctctgcccgccttccctcccgcc (SEQ ID NO:33).

In some embodiments, the DUX4 nucleic acid inhibitor comprises a double-stranded siRNA, wherein the antisense strand of the double-stranded siRNA consists of the nucleobase sequence of any of ttcctctctccatctctgc (SEQ ID NO:23), ttgtcccggaggaaaccgc (SEQ ID NO:24), ttccctgcatgtttccgggtgcccg (SEQ ID NO:25), cttccctgcatgtttccgg (SEQ ID NO:26), ctccgtgggagtcttgagtgtgcca (SEQ ID NO:27), caccccttcatgaatggcg (SEQ ID NO:28), caggctccaccccttcatg (SEQ ID NO:29), ttccgctcaaagcaggctc (SEQ ID NO:30), aaagcgatccttctcaaaggctcgg (SEQ ID NO:31), cctgcgcgggcgccctgcc (SEQ ID NO:32), tatctctgaactaatcatc, or atctctgcccgccttccctcccgcc (SEQ ID NO:33).

In some embodiments, the DUX4 nucleic acid inhibitor comprises a double-stranded siRNA, wherein the antisense of the double-stranded siRNA comprises 16 to 30 linked nucleosides complementary within nucleobases 4295-5840 of SEQ ID NO:3. In some embodiments, the DUX4 nucleic acid inhibitor comprises a double-stranded siRNA, wherein the antisense strand of the double-stranded siRNA comprises a nucleic acid sequence at least 80, 81, 82, 83, 84, 85, 86, 87, 88, 89, 90, 91, 92, 93, 94, 95, 96, 97, 98, 99, or 100% (or any derivable range therein) identical to SEQ ID NO:3.

In some embodiments, the DUX4 nucleic acid inhibitor comprises a double-stranded siRNA, wherein the antisense strand of the double-stranded siRNA comprises a nucleic acid sequence comprising at least, at most, or exactly 10, 11, 12, 13, 14, 15, 16, 17, 18, 19, 20, 21, 22, 23, 24, or 25 (or any derivable range therein) contiguous nucleobases of any of the nucleobase sequences of agcgcctggcggcggaacgcagacc (SEQ ID NO:34), gaaacatgcagggaagggtgcaagc (SEQ ID NO:35), aagactcccacggaggttcagttcc (SEQ ID NO:36), tgcacagtccggctgaggtgcacgg (SEQ ID NO:37), or agaaggatcgctttccaggcatcgc (SEQ ID NO:38).

In some embodiments, the DUX4 nucleic acid inhibitor comprises a double-stranded siRNA, wherein the antisense strand of the double-stranded siRNA consists of the nucleobase sequence of any of agcgcctggcggcggaacgcagacc (SEQ ID NO:34), gaaacatgcagggaagggtgcaagc (SEQ ID NO:35), aagactcccacggaggttcagttcc (SEQ ID NO:36), tgcacagtccggctgaggtgcacgg (SEQ ID NO:37), or agaaggatcgctttccaggcatcgc (SEQ ID NO:38).

In some embodiments, the DUX4 nucleic acid inhibitor comprises a double-stranded siRNA, wherein the sense strand of the double-stranded siRNA comprises a nucleic acid sequence at least 80, 81, 82, 83, 84, 85, 86, 87, 88, 89, 90, 91, 92, 93, 94, 95, 96, 97, 98, 99, or 100% (or any derivable range therein) complementary to the antisense strand of the double-stranded siRNA.

In some embodiments, the DUX4 nucleic acid inhibitor comprises a double-stranded siRNA, wherein the sense strand of the double-stranded siRNA comprises at least, at most, or exactly 1, 2, 3, 4, 5, 6, 7, 8, 9, 10, 11, 12, 13, 14, 15, 16, 17, 18, 19, 20, 21, 22, 23, 24, or 25 (or any derivable range therein) modified nucleosides. In some embodiments, each nucleoside of the sense strand of the double-stranded siRNA comprises a modified nucleoside. In some embodiments, the modified nucleoside is selected from a 2′-F modified nucleoside or a 2′-OMe modified nucleoside. In some embodiments, the DUX4 nucleic acid inhibitor comprises a double-stranded siRNA, wherein the sense strand of the double-stranded siRNA comprises at least at least, at most, or exactly 1, 2, 3, 4, 5, 6, 7, 8, 9, 10, 11, 12, 13, 14, 15, 16, 17, 18, 19, 20, 21, 22, 23, 24, or 25 (or any derivable range therein) modified internucleoside linkages. In some embodiments, each modified internucleoside linkage is a phosphorothioate internucleoside linkage.

In some embodiments, the DUX4 inhibitory nucleic acid provides for at least, at most, or exactly 10, 15, 20, 25, 30, 35, 40, 45, 50, 55, 60, 65, 70, 75, 80, 85, 90, or 95% inhibition of expression of DUX4 in a cell or a cancer cell.

In some embodiments, the nucleic acid inhibitor comprises an siRNA from the Table below:

| | | | | Location of Dux4 | | | |
|---|---|---|---|---|---|---|---|
| | | | | Target | | | |
| | | | | Dux4 | | Dux4 | |
| | Strand of | siRNA | Target | mRNA ² | | genomic ³ | |
| Location | Targeted | Targeted | mRNA or | start | stop | start | stop |
| 1 = tss ¹ | Sequence | Sequence | genomic | site | site | site | site |

siRNAs

| | | | | | | | |
|---|---|---|---|---|---|---|---|
| -1263 | sense | GGCGGGAGGG | genomic | | | 4589 | 4613 |
| | | AAGGCGGGCA | | | | | |
| | | GAGAT (SEQ ID | | | | | |
| | | NO: 39) | | | | | |
| -1247 | sense | GCAGAGAUGG | genomic | | | 4606 | 4624 |
| | | AGAGAGGAA | | | | | |
| | | (SEQ ID NO: 40) | | | | | |
| -1301 | antisense | GGTCTGCGTTC | genomic | | | 4551 | 4527 |
| | | CGCCGCCAGG | | | | | |
| | | CGCT (SEQ ID | | | | | |
| | | NO: 41) | | | | | |
| -921 | sense | GCGGTTTCCTC | genomic | | | 4932 | 4950 |
| | | CGGGACAA | | | | | |
| | | (SEQ ID NO: 42) | | | | | |
| -779 | sense | GGACGACGGA | genomic | | | 5074 | 5092 |
| | | GGCGTGATT | | | | | |
| | | (SEQ ID NO: 43) | | | | | |
| -657 | sense | CGGGCACCCG | genomic | | | 5195 | 5219 |
| | | GAAACATGCA | | | | | |
| | | GGGAA (SEQ ID | | | | | |
| | | NO: 44) | | | | | |
| -650 | sense | CCGGAAACAT | genomic | | | 5202 | 5220 |
| | | GCAGGGAAG | | | | | |
| | | (SEQ ID NO: 45) | | | | | |
| -623 | antisense | GCTTGCACCCT | genomic | | | 5229 | 5205 |
| | | TCCCTGCATGT | | | | | |
| | | TTC (SEQ ID | | | | | |
| | | NO: 46) | | | | | |
| -476 | sense | GAAATGAACG | genomic | | | 5376 | 5394 |
| | | AGAGCCACA | | | | | |
| | | (SEQ ID NO: 47) | | | | | |
| -385 | sense | TGGCACACTCA | genomic | | | 5467 | 5491 |
| | | AGACTCCCACG | | | | | |
| | | GAG (SEQ ID | | | | | |
| | | NO: 48) | | | | | |
| -368 | sense | CCACGGAGGTT | genomic | | | 5484 | 5502 |
| | | CAGTTCCA | | | | | |
| | | (SEQ ID NO: 49) | | | | | |
| -351 | antisense | GGAACTGAAC | genomic | | | 5501 | 5477 |
| | | CTCCGTGGGAG | | | | | |
| | | TCTT (SEQ ID | | | | | |
| | | NO: 50) | | | | | |
| -131 | sense | ACCACCACCAC | genomic | | | 5721 | 5739 |
| | | CACCACCA | | | | | |
| | | (SEQ ID NO: 51) | | | | | |
| -37 | sense | CGCCATTCATG | genomic | | | 5815 | 5833 |
| | | AAGGGGTG | | | | | |
| | | (SEQ ID NO: 52) | | | | | |
| -30 | sense | CAUGAAGGGG | genomic | | | 5822 | 5840 |
| | | TGGAGCCTG | | | | | |
| | | (SEQ ID NO: 53) | | | | | |
| +63 | sense | CCGCAGTGCAC | mRNA (5' | | | 5914 | 5938 |
| | | AGTCCGGCTGA | utr) | | | | |
| | | GGT (SEQ ID | | | | | |
| | | NO: 54) | | | | | |
| +93 | antisense | CCGTGCACCTC | mRNA (5' | | | 5944 | 5920 |
| | | AGCCGGACTGT | utr) | | | | |
| | | GCA (SEQ ID | | | | | |
| | | NO: 55) | | | | | |
| +258 | sense | GAGCCTGCTTT | mRNA | 104 | 122 | 6109 | 6127 |
| | | GAGCGGAA | | | | | |
| | | (SEQ ID NO: 56) | | | | | |
| +484 | sense | CCGAGCCTTTG | mRNA | 330 | 354 | 6335 | 6359 |
| | | AGAAGGATCG | | | | | |
| | | CTTT (SEQ ID | | | | | |
| | | NO: 57) | | | | | |
| +519 | antisense | GCGATGCCTGG | mRNA | 365 | 341 | 6370 | 6346 |
| | | AAAGCGATCCT | | | | | |
| | | TCT (SEQ ID | | | | | |
| | | NO: 58) | | | | | |
| +614 | sense | GGCAGGGCGC | mRNA | 460 | 478 | 6465 | 6483 |
| | | CCGCGCAGG | | | | | |
| | | (SEQ ID NO: 59) | | | | | |
| +1313 | sense | GCGCAACCTCT | mRNA | 1159 | 1177 | 7164 | 7182 |
| | | CCTAGAAA | | | | | |
| | | (SEQ ID NO: 60) | | | | | |
| +1489 | sense | GGGAACACCT | mRNA | 1335 | 1353 | 7339 | 7358 |
| | | GGCTGGCTA | | | | | |
| | | (SEQ ID NO: 61) | | | | | |
| +1676 | sense | TACUGCGCAC | genomic | | | 7527 | 7545 |
| | | GCGCGGGTT | (intron 2) | | | | |
| | | (SEQ ID NO: 62) | | | | | |
| +1741 | sense | CUGCCTCTCCA | genomic | | | 7582 | 7610 |
| | | CCAGCCCA | (intron 2) | | | | |
| | | (SEQ ID NO: 63) | | | | | |
| +2148 | sense | CAAACCTGGAT | mRNA | 1631 | 1649 | 7998 | 8016 |
| | | TAGAGTTA | (exon 3) | | | | |
| | | (SEQ ID NO: 64) | | | | | |
| +2176 | sense | GATGATTAGTT | mRNA | 1659 | 1679 | 8026 | 8044 |
| | | CAGAGATA | | | | | |
| | | (SEQ ID NO: 65) | | | | | |

In some embodiments, the siRNA comprises one of the following nucleic acid sequence: CCGGGCATCGCCACCAGAGAA (SEQ ID NO:66); CAGGGTCCAGATTTGGTTTCA (SEQ ID NO:67); CAGGATTCAGATCTGGTTTCA (SEQ ID NO:68); CAGGCGCAACCTCTCCTAGAA (SEQ ID NO:69); and GAGCCTGCTTTGAGCGGAA (SEQ ID NO:70).

In some embodiments, the nucleic acid inhibitor is at least, at most, or exactly 70, 75, 80, 81, 82, 83, 84, 85, 86, 87, 88, 89, 90, 91, 92, 93, 94, 95, 96, 97, 98, or 99% identical (or any derivable range therein) to a nucleic acid having a sequence of SEQ ID Nos: 5-70. In some embodiments, the nucleic acid inhibitor is at least, at most, or exactly 70, 75, 80, 81, 82, 83, 84, 85, 86, 87, 88, 89, 90, 91, 92, 93, 94, 95, 96, 97, 98, or 99% identical (or any derivable range therein) to a nucleic acid having at least 8, 9, 10, 11, 12, 13, 14, 15, 16, 17, 18, 19 20, 21, 22, 23, 24, 25, 26, 27, 28, 29, or 30 (or any derivable range therein) contiguous nucleic acids of a nucleic acid having the sequence of one of SEQ ID Nos: 5-70.

Other DUX4 nucleic acid inhibitors are known in the art and described in, for example, US20170029814, US20170009230, WO/2016/115490, and US20120225034, which are herein incorporated by reference.

C. DUX4 Inhibitory Antibodies and Polypeptides

In some embodiments, the DUX4 inhibitor comprises an antibody, protein, or polypeptide inhibitor. Exemplary polypeptide inhibitors include Wnt proteins (e.g., recombinant Wnt3a) and DUX4-s, a competitive inhibitor of DUX4. In some embodiments, the DUX4 polypeptide inhibitor comprises a DUX4 polypeptide that lacks transcriptional activity but retains DNA-binding activity. In some embodiments, the DUX4 inhibitor comprises a DUX4-s polypeptide have the following sequence: MALPTPSDSTLPAEARGRGRRRRLVWTPSQSEALRACFERNPYPGIATRERLAQAIGI PEPRVQIWFQNERSRQLRQHRRESRPWPGRRGPPEGRRKRTAVTGSQTALLLRAFEK DRFPGIAAREELARETGLPESRIQIWFQNRRARHPGQGGRAPAQV (SEQ ID NO:4). In some embodiments, the DUX4 inhibitor comprises a polypeptide with at least 75, 80, 81, 82, 83, 84, 85, 86, 87, 88, 89, 90, 91, 92, 93, 94, 95, 96, 97, 98, or 99% identity to SEQ ID NO:4. In some embodiments, the polypeptide inhibitor comprises at least 75, 80, 81, 82, 83, 84, 85, 86, 87, 88, 89, 90, 91, 92, 93, 94, 95, 96, 97, 98, or 99% identity to SEQ ID NO:4 and retains DNA binding activity and/or lacks transcriptional activation activity.

In certain embodiments, an antibody or a fragment thereof that binds to at least a portion of DUX4 protein and inhibits DUX4 activity.

In some embodiments, the anti-DUX4 antibody is a monoclonal antibody or a polyclonal antibody. In some embodiments, the antibody is a chimeric antibody, an affinity matured antibody, a humanized antibody, or a human antibody. In some embodiments, the antibody is an antibody fragment. In some embodiments, the antibody is a Fab, Fab′, Fab′-SH, F(ab′)2, or scFv. In one embodiment, the antibody is a chimeric antibody, for example, an antibody comprising antigen binding sequences from a non-human donor grafted to a heterologous non-human, human or humanized sequence (e.g., framework and/or constant domain sequences). In one embodiment, the non-human donor is a mouse. In one embodiment, an antigen binding sequence is synthetic, e.g., obtained by mutagenesis (e.g., phage display screening, etc.). In one embodiment, a chimeric antibody has murine V regions and human C region. In one embodiment, the murine light chain V region is fused to a human kappa light chain or a human IgG1 C region.

Examples of antibody fragments include, without limitation: (i) the Fab fragment, consisting of VL, VH, CL and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (U.S. Patent Pub. 2005/0214860). Fv, scFv or diabody molecules may be stabilized by the incorporation of disulphide bridges linking the VH and VL domains. Minibodies comprising a scFv joined to a CH3 domain may also be made (Hu et al, 1996).

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with a DUX4 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g., mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, binding fragments and binding domains and CDRs (including engineered forms of any of the foregoing), may be created that are specific to DUX4 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Particularly, the antibodies may be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art. Methods for producing these antibodies are also well known. For example, the following U.S. patents and patent publications provide enabling descriptions of such methods and are herein incorporated by reference: U.S. Patent publication Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent publications, and other publications cited herein and therein are hereby incorporated by reference in the present application.

It is fully expected that antibodies to DUX4 will have the ability to neutralize or counteract the effects of the DUX4 regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into binding fragments having the binding domain or CDR. Removal of the Fc portion reduces the likelihood that the antigen binding fragment will elicit an undesirable immunological response and, thus, antibodies without Fc may be particularly useful for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species.

### III. DUX4-REGULATED GENES

In some embodiments, the DUX4-regulated gene comprises one or more genes listed in the Table below:

| | | | |
|---|---|---|---|
| | Log2 | Log2 | |
| | DUX441 | DUX4-s | |
| Category | Fc* | Fc* | Comments |

Germline and Stem Cells

| | | | |
|---|---|---|---|
| ZSCAN4 | 8.3 | 0.0 | Genome stability, telomere length |
| PRAMEF1 | 8.1 | 0.1 | Melanoma antigen family |
| SPRYD5 | 8.0 | −0.1 | Expressed in oocyte |
| KHDC1L | 8.0 | −0.1 | KH RNA binding domain |
| MBD3L2 | 7.6 | 0.0 | Methyl-CpG-binding protein |
| ZNF705A | 6.8 | −0.1 | Zinc finger protein |
| TRIM43 | 5.8 | 0.0 | Preimplantation embryo |
| TPRX1 | 4.5 | −0.1 | Homeobox protein |
| ZNF217 | 4.1 | −0.3 | Expressed in cancer stem cells |
| HSPA2 | 3.7 | −0.3 | Chaperone, heat shock 70 kd |
| JUP | 3.2 | −0.1 | expressed in germline and testicular cancers |
| FGFR3 | 3.1 | 0.0 | Expressed in spermatogonia |
| CD24 | 2.6 | −0.4 | Stem cell marker |
| SLC2A14 | 2.4 | 0.2 | Spermatogenesis |
| ID2 | 2.3 | 0.3 | Negative regulator of cell differentiation |
| PVRL3 | 2.2 | 0.4 | Spermatid-sertoli junction |
| HOXB2 | 2.2 | 0.0 | Anterior-posterior axis development |
| ZSCAN2 | 2.2 | −0.2 | Spermatogenesis and embryonic development |

RNA Processing

| | | | |
|---|---|---|---|
| SFRS2B | 4.2 | −0.3 | Splicing |
| THOC4 | 4.0 | −0.2 | Splicing, RNA transport |
| ZNHIT6 | 3.5 | 0.3 | sno-RNA processing |
| DBR1 | 3.4 | 0.2 | RNA lariat debranching enzyme |
| TFIP11 | 3.2 | 0.1 | Spliceosome assembly |
| CWC15 | 2.6 | 0.1 | Spliceosome-associated |
| ARS2 | 2.6 | −0.2 | miRNA processing |
| PABPN1 | 2.6 | −0.3 | PolyA binding |
| SFRS17A | 2.5 | 0.2 | Spliceosome-associated |
| RMRP | 2.3 | 0.1 | Mitochondrial RNA processing |
| SNIP1 | 2.1 | −0.2 | miRNA biogenesis |
| RPPH1 | 2.0 | 0.2 | tRNA processing |
| RNGTT | 2.0 | −0.6 | mRNA processing |

Ubiquitin Pathway

| | | | |
|---|---|---|---|
| SIAH1 | 3.7 | −0.1 | Targets TRF2 telomere maintenance |
| FBXO33 | 3.2 | 0.2 | E3 ubiquitin-protein ligase complex |
| PELI1 | 2.9 | 0.1 | E3 ligases involved in innate immunity |
| USP29 | 2.6 | −0.1 | Ubiquitin-specific peptidase |
| ARIH1 | 2.2 | 0.8 | Ubiquitin-conjugating enzyme E2 binding protein |
| TRIM23 | 2.2 | 0.6 | E3 ubiquitin ligase involved in immunity |

Immunity and Innate Defense

| | | | |
|---|---|---|---|
| DEFB103B | 6.4 | 0.1 | Innate defense |
| IFRD1 | 3.0 | −0.2 | Interferon-related developmental regulator |
| CXADR | 2.5 | −0.1 | Leukocyte migration |
| CBARA1 | 2.1 | −0.2 | T-helper 1-mediated autoreactivity |
| SON | 2.1 | −0.3 | Viral response |
| CXCR4 | 2.0 | −0.1 | Chemotaxis |

General Transcription

| | | | |
|---|---|---|---|
| GTF2F1 | 3.2 | 0.3 | General transcription factor IIF |
| MED26 | 2.1 | 0.1 | RNA Pol II mediator complex |
| RRN3 | 2.1 | 0.1 | RNA Poll preinitiation complex |

Cancer Expressed

| | | | |
|---|---|---|---|
| CSAG3 | 5.9 | 0.1 | Chondrosarcoma-associated gene |
| SLC34A2 | 5.5 | 0.0 | Breast cancer biomarker |
| PNMA6B | 3.6 | −0.2 | Paraneoplastic antigen |
| CSElL | 2.9 | 0.1 | Cellular apoptosis susceptibility protein |
| AMACR | 2.7 | 0.1 | Prostate cancer biomarker |

Other

| | | | |
|---|---|---|---|
| FLJ45337 | 3.7 | −0.2 | Endogenous retrovirus |
| HNRNPCL1 | 3.5 | −0.1 | Nucleosome assembly |
| SPTY2D1 | 3.3 | −0.3 | Suppressor of ty retrotransposons in yeast |
| MGC10997 | 2.4 | −0.3 | Endogenous retrotransposon |

One or more DUX4-regulate genes described herein may include at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 genes (or any value or range derivable therein) of genes listed in the table above or DUX4-regulated genes known in the art, such as the DUX4-regulated genes or FSHD biomarkers described in any of Internation Application Publications: WO2013019623, WO2012024535, WO2013192185, and WO2015143062, which are incorporated by reference.

### IV. IMMUNOTHERAPY

Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

A. Checkpoint Inhibitors

An “immune checkpoint inhibitor” is any molecule that directly or indirectly inhibits, partially or completely, an immune checkpoint pathway. Without wishing to be bound by any particular theory, it is generally thought that immune checkpoint pathways function to turn on or off aspects of the immune system, particularly T cells. Following activation of a T cell, a number of inhibitory receptors can be unregulated and present on the surface of the T cell in order to suppress the immune response at the appropriate time. In the case of persistent immune stimulation, such as with chronic viral infection, for example, immune checkpoint pathways can suppress the immune response and lead to immune exhaustion. Examples of immune checkpoint pathways include, without limitation, PD-1 (Programmed cell death protein 1)/PD-L1 (Programmed death-ligand 1), CTLA4 (cytotoxic T-lymphocyte-associated protein 4)/B7-1 (Cluster of differentiation 80), TIM-3 (T-cell immunoglobulin and mucin-domain containing-3), LAG-3 (Lymphocyte-activation gene 3), IDO1 (indolaimine-2, 3-deoxygenase 1), CD276 and VTCN1. In the instance of the PD-1/PD-L1 immune checkpoint pathway, an inhibitor may bind to PD-1 or to PD-L1 and prevent interaction between the receptor and ligand. Therefore, the inhibitor may be an anti-PD-1 antibody or anti-PD-L1 antibody. Similarly, in the instance of the CTLA4/B7-1 immune checkpoint pathway, an inhibitor may bind to CTLA4 or to B7-1 and prevent interaction between the receptor and ligand. In some embodiments, the checkpoint inhibitor comprises an inhibitor of CTLA-4, PD-1, PD-L1, PD-L2, LAG-3, TIM-3, VISTA (V-domain Ig suppressor of T cell activation), TIGIT (T cell immunoreceptor with Ig and ITIM domains), IDO1, BTLA (B- and T-lymphocyte attenuator), or combinations thereof. Inhibitors include antibodies, polypeptides, compounds, and nucleic acids that target and inhibit the molecule.

Further examples of immune checkpoint inhibitors can be found, for example, in WO2014/144885. Such immune checkpoint inhibitors are incorporated by reference herein. In some embodiments of any one of the methods, compositions or kits provided, the immune checkpoint inhibitor is a small molecule inhibitor of an immune checkpoint pathway. In some embodiments of any one of the methods, compositions or kits provided, the immune checkpoint inhibitor is a polypeptide that inhibits an immune checkpoint pathway. In some embodiments of any one of the methods, compositions or kits provided, the inhibitor is a fusion protein. In some embodiments of any one of the methods, compositions or kits provided, the immune checkpoint inhibitor is an antibody. In some embodiments of any one of the methods, compositions or kits provided, the antibody is a monoclonal antibody.

Non-limiting examples of immune checkpoint inhibitors include fully human monoclonal antibodies, such as RG7446, BMS-936558/MDX-1106, BMS-936559 (anti-PD-L1 antibody), Yervoy/ipilimumab (anti-CTLA-4 checkpoint inhibitor), and Tremelimumab (CTLA-4 blocking antibody); humanized antibodies, such as pidilizumab (CT-011, CureTech Ltd.) and lambrolizumab (MK-3475, Merck, PD-1 blocker); and fusion proteins, such as AMP-224 (Merck). Other examples of checkpoint inhibitors include PD-L1 monoclonal Antibody (Anti-B7-H1; MEDI4736), Nivolumab (BMS-936558, Bristol-Myers Squibb, anti-PD1 antibody), CT-011 (anti-PD1 antibody), BY55 monoclonal antibody, MPLDL3280A (anti-PD-L1 antibody), and MSB0010718C (anti-PD-L1 antibody), MDX-1105 (Medarex), and MPDL3280A (Genentech). Anti-KIR antibodies such as lirlumab (Innate Pharma) and IPH2101 (Innate Pharma) may perform similar functions in NK cells. Further examples of checkpoint inhibitors include Atezolizumab (Tecentriq), a humanized PD-L1 antibody, Avelumab (Bavencio), a human PD-L1 antibody, Pembrolizumab (Keytruda), a humanized PD-1 antibody, and Durvalumab (Imfinzi), a human PD-L1 antibody. In some embodiments, the checkpoint inhibitor comprises Atezolizumab, Avelumab, Ipilimumab, Nivolumab, Pembrolizumab, or Durvalumab. Further examples of checkpoint inibitors include TIM-3 antibodies such as LY3321367 (Eli Lilly and Company), MBG453 (Novartis Pharmaceuticals), and TSR-022 (Tesaro) and VISTA antibody such as JNJ-61610588.

B. Agonists of Co-Stimulatory Molecules

In some embodiments, the immunotherapy comprises an agonist of a co-stimulatory molecule. In some embodiments, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include antibodies, polypeptides, compounds, and nucleic acids.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

E. Antibody Therapy

Exemplary antibody therapies include Alemtuzumab (Campeth-1H), an anti-CD52 humanized IgG1 monoclonal antibody, Durvalumab (Imfinzi), a human immunoglobulin G1 kappa (IgGlκ) monoclonal antibody that blocks the interaction of programmed cell death ligand 1 (PD-L1) with the PD-1 and CD80 (B7.1) molecules, Ofatumumab, a second generation human IgG1 antibody that binds to CD20, Rituximab, a chimeric monoclonal IgG1 antibody specific for CD20, anti-CD47 therapy, anti-GD2 antibodies.

F. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNδ).

Interleukins have an array of immune system effects. IL-2 is an exemplary interleukin cytokine therapy.

G. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

H. Oncolytic Virus

An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

I. Polysaccharides

Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

J. Neoantigens

Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### V. ADDITIONAL THERAPIES

In certain embodiments, the patient is administered an additional therapy or a prior cancer treatment. In some embodiments, the additional therapy or prior cancer treatment comprises a checkpoint inhibitor, which are described herein. In some embodiments, the additional therapy or prior cancer treatment comprises a conventional cancer therapy, which is described in this section.

Conventional cancer therapies include chemotherapies, radiation based treatments, and surgery. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### VI. PHARMACEUTICAL COMPOSITIONS

Methods and compositions may be provided for the treatment of cancer. In certain embodiments, there may be provided methods and compositions involving pharmaceutical compositions that comprise one or more therapeutic agents as described herein.

The therapeutic agents useful in the methods may be in the form of free acids, free bases, or pharmaceutically acceptable addition salts thereof. Such salts can be readily prepared by treating the agents with an appropriate acid. Such acids include, by way of example and not limitation, inorganic acids such as hydrohalic acids (hydrochloric, hydrobromic, hydrofluoric, etc.), sulfuric acid, nitric acid, and phosphoric acid, and organic acids such as acetic acid, propanoic acid, 2-hydroxyacetic acid, 2-hydroxypropanoic acid, 2-oxopropanoic acid, propandioic acid, and butandioic acid. Conversely, the salt can be converted into the free base form by treatment with alkali.

Aqueous compositions in some aspects comprise an effective amount of the therapeutic agent, further dispersed in pharmaceutically acceptable carrier or aqueous medium. The phrase “pharmaceutically or pharmacologically acceptable” refer to compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the pharmaceutical compositions is contemplated. Supplementary active ingredients also can be incorporated into the compositions.

Solutions of pharmaceutical compositions can be prepared in water suitably mixed with a surfactant, such as hydroxypropylcellulose. Dispersions also can be prepared in glycerol, liquid polyethylene glycols, mixtures thereof, and in oils. Under ordinary conditions of storage and use, these preparations contain a preservative to prevent the growth of microorganisms.

The pharmaceutical compositions may be administered in the form of injectable compositions either as liquid solutions or suspensions; solid forms suitable for solution in, or suspension in, liquid prior to injection may also be prepared. These preparations also may be emulsified. For instance, the composition may contain at least about, at most about, or about 1, 5, 10, 25, 50 mg or up to about 100 mg of human serum albumin per milliliter of phosphate buffered saline. Other pharmaceutically acceptable carriers include aqueous solutions, non-aqueous solvents, non-toxic excipients, including salts, preservatives, buffers and the like.

Examples of non-aqueous solvents include propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

Administration of pharmaceutical compositions may be via any common route so long as the target tissue, cell or intracellular department is available via that route. This includes oral, nasal, buccal, rectal, vaginal or topical. Alternatively, administration may be by orthotopic, intradermal subcutaneous, intramuscular, intraperitoneal or intravenous injection. Such compositions would normally be administered as pharmaceutically acceptable compositions that include physiologically acceptable carriers, buffers or other excipients. Volume of an aerosol may be between about 0.01 mL and 0.5 mL.

Additional formulations may be suitable for oral administration. “Oral administration” as used herein refers to any form of delivery of a therapeutic agent or composition thereof to a subject wherein the agent or composition is placed in the mouth of the subject, whether or not the agent or composition is swallowed. Thus, “Oral administration” includes buccal and sublingual as well as esophageal administration. Absorption of the agent can occur in any part or parts of the gastrointestinal tract including the mouth, esophagus, stomach, duodenum, ileum and colon. Oral formulations include such typical excipients as, for example, pharmaceutical grades of mannitol, lactose, starch, magnesium stearate, sodium saccharine, cellulose, magnesium carbonate and the like. The compositions may take the form of solutions, suspensions, tablets, pills, capsules, sustained release formulations or powders.

In one embodiment, the oral formulation can comprise the therapeutic agent and one or more bulking agents. Suitable bulking agents are any such agent that is compatible with the therapeutic agent including, for example, lactose, microcrystalline cellulose, and non-reducing sugars, such as mannitol, xylitol, and sorbitol. One example of a suitable oral formulation includes spray-dried therapeutic agent-containing polymer nanoparticles (e.g., spray-dried poly(lactide-co-glycolide)/amifostine nanoparticles having a mean diameter of between about 150 nm and 450 nm; see Pamujula, et al., 2004, which is here by incorporated by reference in its entirety). The nanoparticles can contain between about 20 and 50 w/w % therapeutic agent for example, between about 25% and 50%.

In some embodiments, when the route is topical, the form may be a cream, ointment, salve or spray. Topical formulations may include solvents such as, but not limited to, dimethyl sulfoxide, water, N,N-dimethylformamide, propylene glycol, 2-pyrrolidone, methyl-2-pyrrolidone, and/or N-methylforamide. To enhance skin permeability, if necessary, the skin area to be treated can be pre-treated with dimethylsulfoxide; see Lamperti et al., 1990, which is hereby incorporated by reference in its entirety.

In other embodiments, the pharmaceutical compositions may be for subcutaneous administration (e.g., injection and/or implantation). For example, implantable forms may be useful for patients which are expected to undergo multiple CT scans over an extended period of time (e.g., one week, two weeks, one month, etc.). In one example, such subcutaneous forms can comprise the therapeutic agent and a carrier, such as a polymer. The polymers may be suitable for immediate or extended release depending on the intended use. In one example, the therapeutic agent can be combined with a biodegradable polymer (e.g., polylactide, polyglycolide, and/or a copolymers thereof). In another example, subcutaneous forms can comprise a microencapsulated form of the therapeutic agent, see, e.g., Srinivasan et al., 2002, which is hereby incorporated by reference in its entirety. Such microencapsulated forms may comprise the therapeutic agent and one or more surfactant and other excipients (e.g., lactose, sellulose, cholesterol, and phosphate- and/or stearate-based surfactants).

In a further embodiment, the therapeutic agent or pharmaceutical compositions may be administered transdermally through the use of an adhesive patch that is placed on the skin to deliver the therapeutic agent through the skin and into the bloodstream. An advantage of the transdermal drug delivery route relative to other delivery systems such as oral, topical, or intravenous is that the patch provides a controlled release of the therapeutic agent into the patient, usually through a porous membrane covering a reservoir of the therapeutic agent or through body heat melting thin layers of therapeutic agent embedded in the adhesive. In practicing certain aspects, any suitable transdermal patch system may be used, including, without limitation, single-layer drug-in-adhesive, multi-layer drug-in-adhesive, and reservoir.

An effective amount of the pharmaceutical composition may be determined based on the intended goal, such as treating cancer, or inducing apoptosis or inhibiting cell proliferation. The term “unit dose” or “dosage” refers to physically discrete units suitable for use in a subject, each unit containing a predetermined quantity of the therapeutic agent calculated to produce the desired responses, discussed above, in association with its administration, i.e., the appropriate route and treatment regimen.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. METHODS OF TREATMENT

Compositions or methods described herein may be administered to any patient that have DUX4-expressing cancer cells for the treatment of cancer.

A. Treatment of Cancer

The cancer may be a solid tumor, metastatic cancer, or non-metastatic cancer. In certain embodiments, the cancer may originate in the bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus.

The cancer may specifically be of the following histological type, though it is not limited to these: neoplasm, malignant; carcinoma; undifferentiated, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophogogastric, glioblastoma, adrencorical, vueal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The term “treatment” or “treating” means any treatment of a disease in a mammal, including:

(i) preventing the disease, that is, causing the clinical symptoms of the disease not to develop by administration of a protective composition prior to the induction of the disease;

(ii) suppressing the disease, that is, causing the clinical symptoms of the disease not to develop by administration of a protective composition after the inductive event but prior to the clinical appearance or reappearance of the disease;

(iii) inhibiting the disease, that is, arresting the development of clinical symptoms by administration of a protective composition after their initial appearance; and/or

(iv) relieving the disease, that is, causing the regression of clinical symptoms by administration of a protective composition after their initial appearance.

The selected treatment regimen can be an aggressive one which is expected to result in the best clinical outcome (e.g., complete cure of the disease) or a more moderate one which may relieve symptoms of the disease yet results in incomplete cure of the disease. The type of treatment can include a surgical intervention, administration of a therapeutic drug such as a DUX4 inhibitor or immunotherapy, an exposure to radiation therapy and/or any combination thereof. The dosage, schedule and duration of treatment can vary, depending on the severity of disease and the selected type of treatment, and those of skill in the art are capable of adjusting the type of treatment with the dosage, schedule and duration of treatment.

Biomarkers like DUX4 that can predict the efficacy of certain therapeutic regimen and can be used to identify patients who will receive benefit of a conventional single or combined modality therapy before treatment begins or to modify or design a future treatment plan after treatment. In the same way, those patients who do not receive much benefit from such conventional single or combined modality therapy and can offer them alternative treatment(s) may be identified.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer metastasis in patients determined to have DUX4-expressing cancers.

B. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1—specificity). The ROC curve is thus the sensitivity as a function of fall-out. In general, if the probability distributions for both detection and false alarm are known, the ROC curve can be generated by plotting the cumulative distribution function (area under the probability distribution from −infinity to + infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety.

ROC analysis is useful for determining cut-off values for expression levels, protein levels, or activity levels. Such cut-off values can be used to determine a patient's prognosis and to predict a patient's response to a particular therapy.

C. Combination Therapy

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent, chemotherapeutic, or checkpoint inhibitor described herein or known in the art is “B”:

| | | | | | | | | | |
|---|---|---|---|---|---|---|---|---|---|
| A/B/A | B/A/B | B/B/A | A/A/B | A/B/B | B/A/A | A/B/B/B | B/A/B/B | B/B/B/A | B/B/A/B |
| A/A/B/B | A/B/A/B | A/B/B/A | B/B/A/A | B/A/B/A | B/A/A/B | A/A/A/B | B/A/A/A | A/B/A/A | A/A/B/A |

In some embodiments, more than one course of therapy may be employed. It is contemplated that multiple courses may be implemented.

### VIII. SAMPLES

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from cancerous tissue by any of the biopsy methods previously mentioned. In other embodiments the sample may be obtained from any of the tissues provided herein that include but are not limited to gall bladder, skin, heart, lung, breast, pancreas, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from cystic fluid or fluid derived from a tumor or neoplasm. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, scraping, phlebotomy, or any other methods known in the art. In some cases, the sample may be obtained, stored, or transported using components of a kit of the present methods. In some cases, multiple samples may be obtained for diagnosis by the methods described herein. In other cases, multiple samples, such as one or more samples from one tissue type (for example colon) and one or more samples from another tissue (for example buccal) may be obtained for diagnosis by the methods. In some cases, multiple samples such as one or more samples from one tissue type (e.g. rectal) and one or more samples from another tissue (e.g. cecum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm or of a suspected tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IX. ANALYSIS OF GENE EXPRESSION

A gene shall be understood to be specifically expressed in a certain cell type if the expression level of said gene in said cell type is at least 2-fold, 5-fold, 10-fold, 100-fold, 1000-fold, or 10000-fold higher than in a reference cell type, or in a mixture of reference cell types. Reference cell types include non-cancerous tissue cells or a heterogeneous population of cancers.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

In certain aspects, the determination of expression levels is on a gene chip, such as an Affymetrix™ gene chip. In another aspect, the determination of expression levels is done by kinetic real time PCR.

In certain aspects, the methods can relate to a system for performing such methods, the system comprising (a) apparatus or device for storing data on expression levels or nodal status of the patient; (b) apparatus or device for determining the expression level of at least one marker gene or activity; (c) apparatus or device for comparing the expression level of the first marker gene or activity with a predetermined first threshold value; (d) apparatus or device for determining the expression level of at least one second, third, fourth, 5^{th}, 6^{th} or more marker gene or activity and for comparing with a corresponding predetermined threshold; and (e) computing apparatus or device programmed to provide a unfavorable or poor prognosis or favorable prognosis based on the comparisons or a predicted therapeutic response.

The person skilled in the art readily appreciates that an unfavorable or poor prognosis can be given if the expression level of the first marker gene with the predetermined first threshold value indicates a tumor that is likely to recur or not respond well to standard therapies.

The expression patterns can also be compared by using one or more ratios between the expression levels of different cancer biomarkers. Other suitable measures or indicators can also be employed for assessing the relationship or difference between different expression patterns.

The expression levels of cancer biomarkers can be compared to reference expression levels using various methods. These reference levels can be determined using expression levels of a reference based on all cancer patients. Alternatively, it can be based on an internal reference such as a gene that is expressed in all cells. In some embodiments, the reference is a gene expressed in cancer cells at a higher level than any biomarker. Any comparison can be performed using the fold change or the absolute difference between the expression levels to be compared. One or more cancer biomarkers can be used in the comparison. It is contemplated that 1, 2, 3, 4, 5, 6, 7, 8, 9, 10, and/or 11 biomarkers (or any range derivable therein) may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. In some embodiments, patients with a poor prognosis have a relatively high level of expression (overexpression) or relatively low level of expression (underexpression) when compared to patients with a better or favorable prognosis, or vice versa.

Fold increases or decreases may be, be at least, or be at most 1-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some embodiments, the levels can be relative to a non-metastatic control or relative to a metastatic control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some embodiments.

Any biological sample from the patient that contains cancer cells may be used to evaluate the expression pattern of any biomarker discussed herein. In some embodiments, a biological sample from a tumor is used. Evaluation of the sample may involve, though it need not involve, panning (enriching) for cancer cells or isolating the cancer cells.

A. Measurement of Gene Expression Using Nucleic Acids

Testing methods based on differentially expressed gene products are well known in the art. In accordance with one aspect, the differential expression patterns of cancer biomarkers can be determined by measuring the levels of RNA transcripts of these genes, or genes whose expression is modulated by the these genes, in the patient's cancer cells. Suitable methods for this purpose include, but are not limited to, quantitative RT-PCR, RNA-seq, Northern Blot, in situ hybridization, Southern Blot, slot-blotting, nuclease protection assay and oligonucleotide arrays.

In certain aspects, RNA isolated from cancer cells can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some embodiments, detection may involve fluorescence resonance energy transfer (FRET) or some other kind of quantum dots.

Amplification primers or hybridization probes for a cancer biomarker can be prepared from the gene sequence or obtained through commercial sources, such as Affymatrix. In certain embodiments the gene sequence is identical or complementary to at least 8 contiguous nucleotides of the coding sequence.

Sequences suitable for making probes/primers for the detection of their corresponding cancer biomarkers include those that are identical or complementary to all or part of the cancer biomarker genes described herein. These sequences are all nucleic acid sequences of cancer biomarkers.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. In some embodiments, because each of the biomarkers has more than one human sequence, it is contemplated that probes and primers may be designed for use with each of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In another embodiment, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one embodiment, quantitative RT-PCR (such as TaqMan, ABI) is used for detecting and comparing the levels of RNA transcripts in cancer samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of cancer biomarkers in cancer cells. The probes suitable for detecting the corresponding cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be 50, 100, 200, 300, 400, 500 or more probes/cm2.

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods and the arrays are not limited in its utility with respect to any parameter except that the probes detect expression levels; consequently, methods and compositions may be used with a variety of different types of genes. In some examples, the microarray is a tissue microarray which contains many small representative tissue samples from hundreds of different cases assembled on a single histologic slide, and allows high throughput analysis of multiple specimens at the same time (Wilczynski Modern Surgical Pathology 2^{nd} Edition 2009).

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In one embodiment, nuclease protection assays are used to quantify RNAs derived from the cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

B. Measurement of Gene Expression Using Proteins and Polypeptides

In other embodiments, the differential expression patterns of cancer biomarkers can be determined by measuring the levels of polypeptides encoded by these genes in cancer cells. Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the cancer biomarker genes described herein.

One example of a method suitable for detecting the levels of target proteins in peripheral blood samples is ELISA. In an exemplifying ELISA, antibodies capable of binding to the target proteins encoded by one or more cancer biomarker genes are immobilized onto a selected surface exhibiting protein affinity, such as wells in a polystyrene or polyvinylchloride microtiter plate. Then, cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another ELISA embodiment, the cancer cell samples containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 49° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, 1¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Various protocols for conducting RIA to measure the levels of polypeptides in cancer cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as a cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In other aspects, the levels of polypeptides in samples can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

### X. KITS

Certain aspects of the disclosure also encompass kits for performing the methods of the disclosure, such as detection of, diagnosis of, or treatment of DUX4+ cancers. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

In a particular aspect, these kits may comprise a plurality of agents for assessing the expression of DUX4, wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit embodiments relate to kits comprising the therapeutic compositions of the disclosure. The kits may be useful in the treatment methods of the disclosure and comprise instructions for use.

### XI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—DUX4 Suppresses MHC Class I to Promote Cancer Immune Evasion and Resistance to Checkpoint Blockade

Advances in cancer immunotherapies make it critical to identify genes that modulate antigen presentation and tumor-immune interactions. The inventors report that DUX4, an early embryonic transcription factor that is normally silenced in somatic tissues, is re-expressed in diverse solid cancers. Both cis-acting inherited genetic variation and somatically acquired mutations in trans-acting repressors contribute to DUX4 re-expression in cancer. Although many DUX4 target genes encode self-antigens, DUX4-expressing cancers were paradoxically characterized by reduced markers of anti-tumor cytolytic activity and lower MHC Class I gene expression. The inventors demonstrate that DUX4 expression blocks interferon-γ-mediated induction of MHC Class I, implicating suppressed antigen presentation in DUX4-mediated immune evasion. Clinical data in metastatic melanoma confirmed that DUX4 expression was associated with significantly reduced progression-free and overall survival in response to anti-CTLA-4. The inventors' results demonstrate that cancers can escape immune surveillance by reactivating a normal developmental pathway and identify a therapeutically relevant mechanism of cell-intrinsic immune evasion.

A. Introduction

Immune checkpoint blockade therapies, which act on T cell inhibitory receptors including CTLA-4 and PD-1, induce durable responses across diverse cancers. However, a majority of patients do not respond to these therapies, and initially responsive cancers may relapse (Ribas and Wolchok, 2018; Sharma et al., 2017; Topalian et al., 2015). Identifying molecular mechanisms that influence therapeutic response and relapse is critical in order to realize the full therapeutic potential of checkpoint blockade.

The efficacy of checkpoint blockade relies upon cytotoxic T cell recognition of antigens presented by MHC Class I on malignant cells. As a consequence, genetic lesions that suppress antigen presentation or blunt tumor-immune interactions can permit malignant cells to evade cytotoxic T cells. The inventors performed a pan-cancer analysis of tumor transcriptomes in order to identify potential regulators of tumor-immune interactions. The inventors sought to identify genes whose expression was restricted to cancers or immune-privileged sites such as the testes and early embryo. While such approaches have been historically used to identify cancer-testis (CT) antigens—proteins whose expression is normally restricted to the embryo and/or germ cells, but which can become re-expressed and antigenic in cancer (Caballero and Chen, 2009)—the inventors hypothesized that such a search might also reveal regulators of antigen presentation and immune modulation. The inventors therefore undertook an unbiased search for such genes using the transcriptomes of 9,759 samples from 33 distinct cancer types, 704 associated peritumoral normal samples, and 34 tissues from healthy individuals. The inventors' analysis revealed that DUX4, an early embryonic transcription factor that is normally silenced in somatic tissues, is re-expressed in many solid cancer types. DUX4 re-expression in cancer results in suppression of MHC Class I-dependent antigen presentation, immune evasion, and resistance to immune checkpoint blockade.

B. Results

1. Large-Scale Identification of Genes with Cancer-Specific Expression Patterns

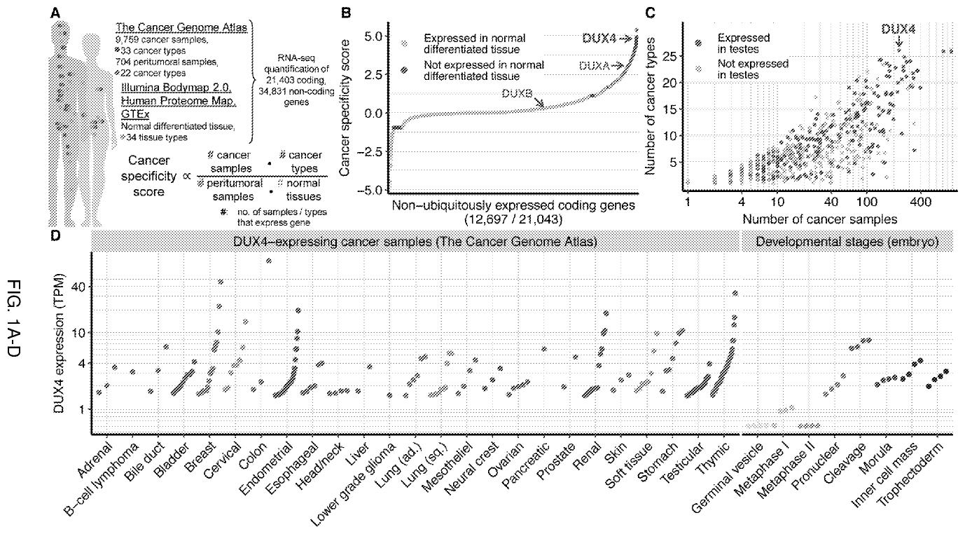

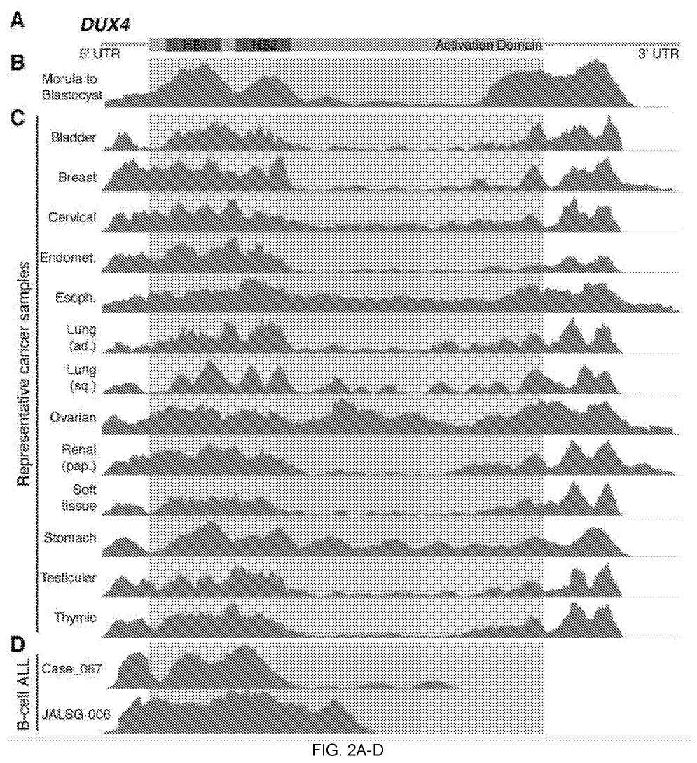

The inventors sought to identify genes that were expressed in multiple cancer types, but not in corresponding peritumoral normal tissues or other somatic tissues isolated from healthy individuals. The inventors compared the transcriptomes of 9,759 cancer samples from 33 distinct cancers (The Cancer Genome Atlas, TCGA) to the transcriptomes of 34 normal tissues, including peritumoral normal tissues (TCGA) and somatic tissues of healthy individuals (Illumina Human Body Map 2.0 and GTEx), as well as protein-level estimates from the Human Proteome Map (Kim et al., 2014; Kosti et al., 2016). The inventors computed a quantitative measure of cancer-specific expression for each gene that was proportional to the numbers of cancer samples and types in which the gene was expressed and inversely proportional to the numbers of peritumoral normal samples and other healthy somatic tissues exhibiting detectable expression of the gene (FIG. 1A).

The inventors' quantitative cancer-specific expression score allowed them to rank each gene according to its relative level of expression in malignant versus normal somatic tissue (FIG. 1B, 7A). The inventors' analysis highlighted many genes with known roles in tumorigenesis, including OCT4 pseudogenes (Hayashi et al., 2013) and genes that are recurrently translocated in cancer, such as TLX3 and members of the SSX gene family (Smith and McNeel, 2010). Many genes that exhibited the most cancer-specific expression patterns encoded known CT antigens, including members of the GAGE, MAGE, PAGE, PRAME, and SPANX gene families.

Given that CT antigens were strongly enriched among the most cancer-specific genes, the inventors tested whether other classes of genes were preferentially expressed in cancers. The inventors performed a Gene Ontology (GO)-based comparison of the 500 highest-scoring genes against a background set of non-ubiquitously expressed genes. The inventors used non-ubiquitously expressed genes as a background set in order to avoid confounding their analysis with housekeeping genes. Genes involved in spermatogenesis comprised ˜5% of the most cancer-specific genes (False Discovery Rate (FDR)<10-3), consistent with the inventors' identification of many CT antigens. Unexpectedly, the most enriched biological pathway was transcriptional regulation (FDR<10-5), which encompassed 19% of the highest-ranked genes. 28 of the 500 highest-ranked genes encode sequence-specific transcription factors, many of which are normally expressed only in germ cells or the embryo. Each of these factors could potentially influence tumor-immune interactions by modulating CT antigen expression, antigen presentation, or interferon signaling.

2. DUX4 is Re-Expressed in Diverse Cancers

Three genes (CGBS, SMC1B, and DUX4) exhibited the strongest pan-cancer signals. Expressed in testes but not in any queried somatic tissues, they each were also expressed in solid cancers arising from 26 distinct tissue types (FIG. 1B-C, 7B). SMC1B encodes a meiosis-specific subunit of cohesin (Revenkova et al., 2001); CGBS encodes a subunit of chorionic gonadotropin; DUX4 encodes an early embryonic transcription factor (De Iaco et al., 2017; Hendrickson et al., 2017; Snider et al., 2010; Whiddon et al., 2017). Chorionic gonadotropin promotes maternal immunotolerance and is a biomarker of cancer (Kayisli et al., 2003; Stenman et al., 2004), suggesting that the inventors' ranking of cancer-specific genes enriched for potential mediators of tumor-immune interactions.

DUX4 was a particularly intriguing candidate for modulating tumor-immune interactions. DUX4 encodes a double homeobox transcription factor that acts as a pioneer factor demarcating the two-cell “cleavage” stage of early embryogenesis, after which it is epigenetically repressed (De Iaco et al., 2017; Hendrickson et al., 2017; Snider et al., 2010; Whiddon et al., 2017). DUX4 is expressed at low levels in the immune-privileged sites of the testis and thymus, but otherwise silenced in somatic tissues (Das and Chadwick, 2016; Snider et al., 2010). Several genes that are direct transcriptional targets of DUX4, such as PRAME genes, encode CT antigens that were first identified based on their cancer-specific expression and immunogenic potential (Chang et al., 2011; Ikeda et al., 1997). DUX4 was commonly expressed in cancers of the bladder, breast, cervix, endometrium, esophagus, lung, ovary, kidney, soft tissue, and stomach, and most commonly expressed in testicular germ cell cancers and thymomas (FIG. 1D).

3. DUX4 is Expressed at Physiological Levels as a Full-Length mRNA

Since DUX4 is normally silenced in somatic cells, the inventors first tested whether DUX4 was expressed at potentially physiologically relevant levels. The inventors compared DUX4 mRNA levels in DUX4-expressing (DUX4+) cancer samples to DUX4 mRNA levels during early embryogenesis, including the cleavage stage, whose transcriptional program is driven by DUX4. DUX4 was typically expressed at levels ranging from ˜2-10 transcripts per million (TPM) in DUX4+ cancer samples, comparable to its endogenous expression during embryogenesis (FIG. 1D).

The inventors next confirmed that cancers expressed a full-length transcript encoding the complete DUX4 transcription factor. Testing for full-length DUX4 mRNA expression was important for three reasons. First, alternative splicing generates multiple DUX4 isoforms, of which only the longest isoform includes the DUX4 C-terminal transcription activation domain (Geng et al., 2012; Snider et al., 2010). Second, DUX4 is recurrently translocated in round-cell sarcoma and B-cell acute lymphoblastic leukemia (B-ALL) to the CIC and IGH loci, generating fusion proteins containing N- and C-terminal truncations of DUX4, respectively (Kawamura-Saito et al., 2006; Lilljebjorn et al., 2016; Liu et al., 2016; Yasuda et al., 2016; Zhang et al., 2016). Because DUX4 requires both its N-terminal DNA-binding domains and its C-terminal activation domain to activate its target genes (Choi et al., 2016), neither fusion protein preserves endogenous DUX4 function. Third, DUX4 exhibits high sequence homology to its paralog DUX4C, such that errors in short read alignment could potentially confound estimates of DUX4 versus DUX4C mRNA levels.

The inventors assessed DUX4 alternative splicing by testing whether the long or short isoform of DUX4 was preferentially expressed in cancers. The inventors identified spliced reads that unambiguously distinguished between the two isoforms in approximately one-third of DUX4+ samples. Only four DUX4+ samples exhibited any evidence of short isoform expression, and in each case, the inventors observed only one or two reads supporting the short isoform (data not shown). The vast majority of expressed DUX4 mRNA arose from the long isoform containing the complete open reading frame.

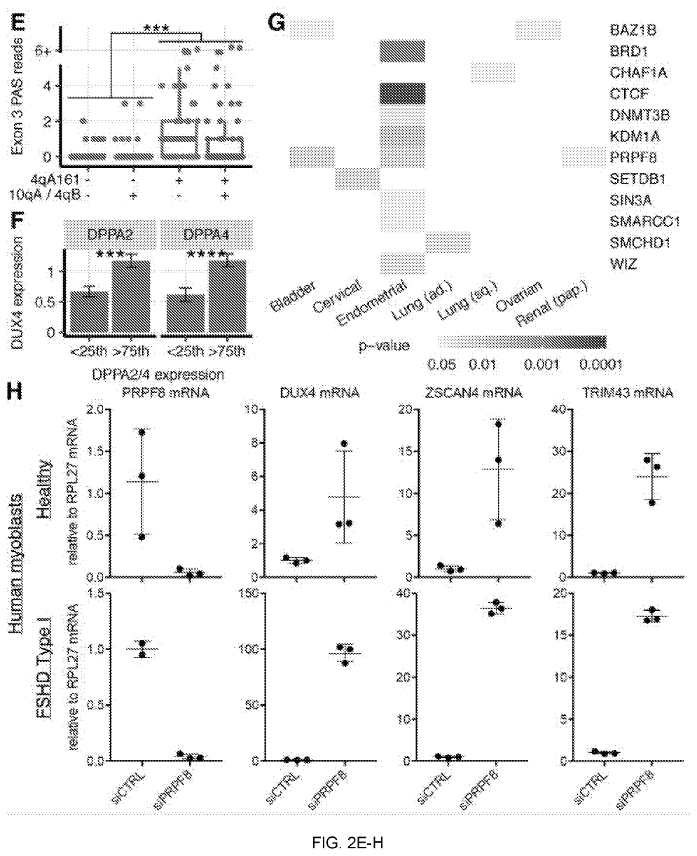

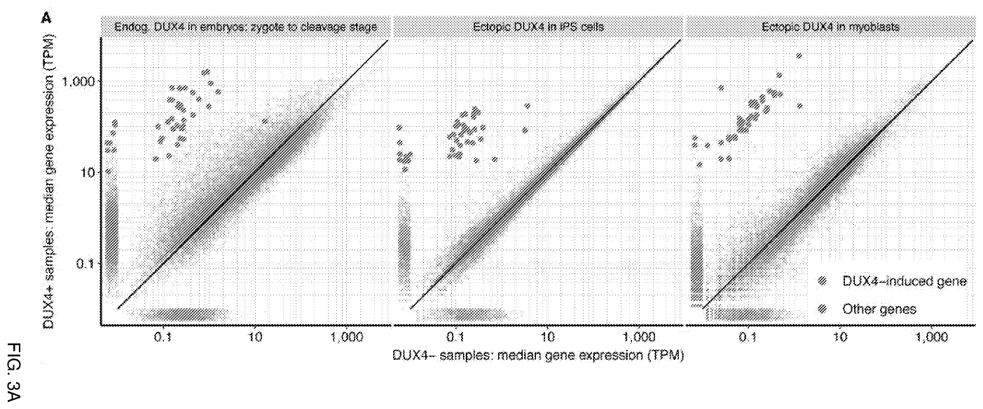

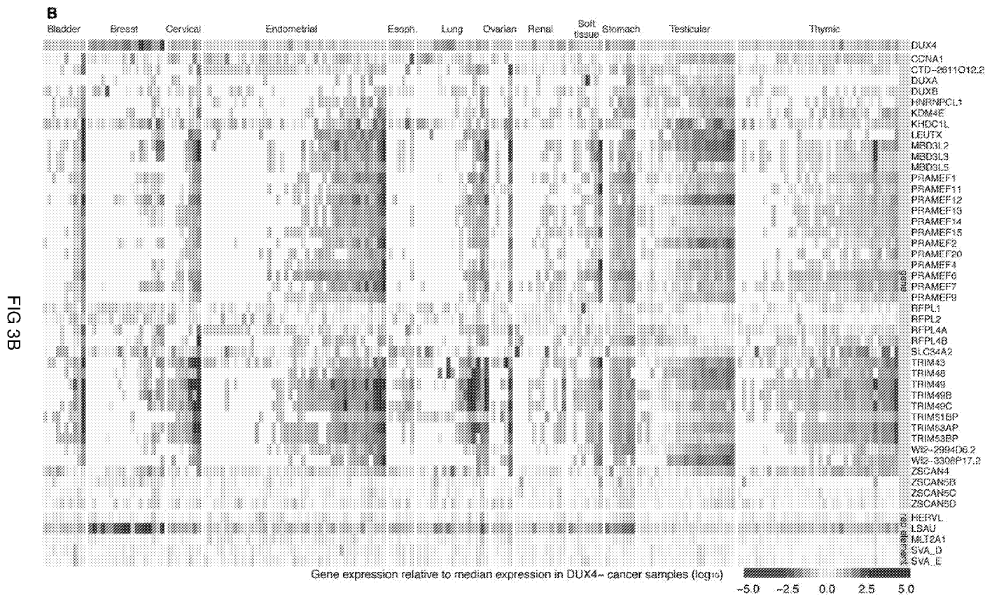

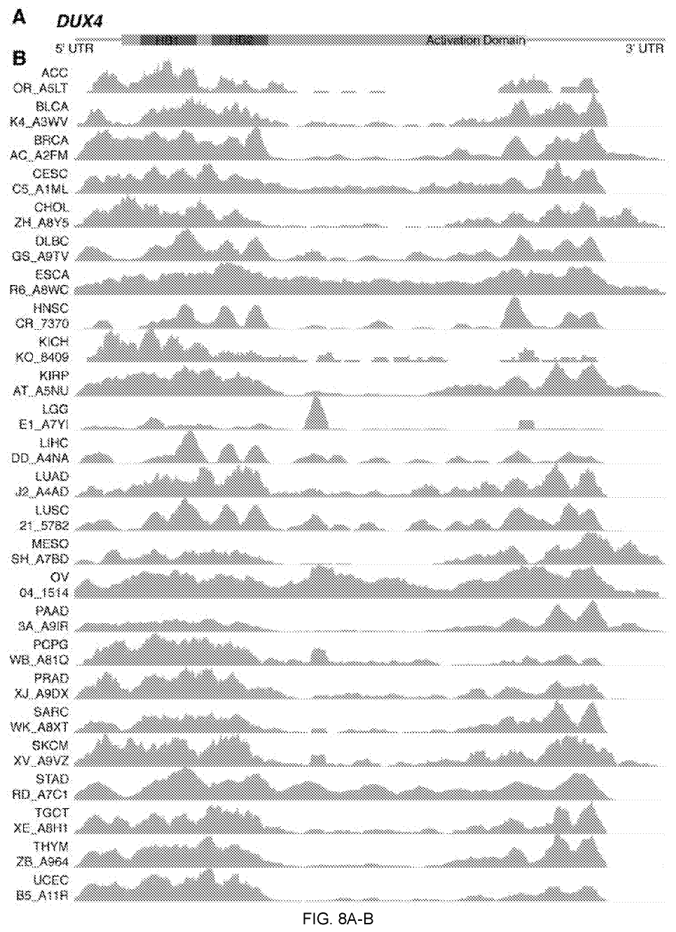

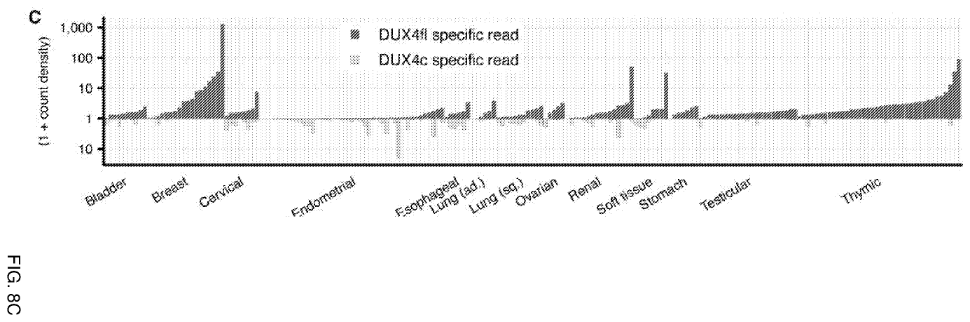

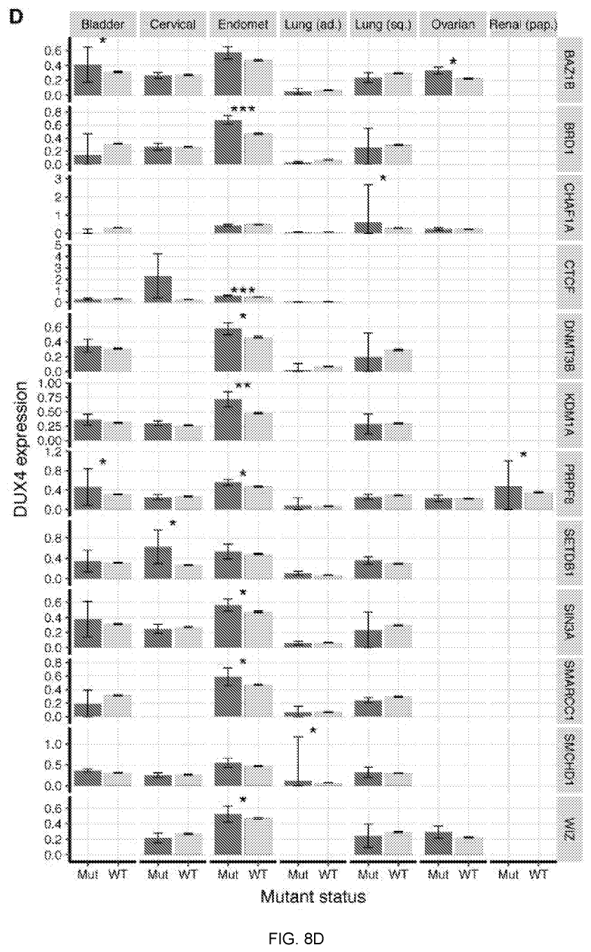

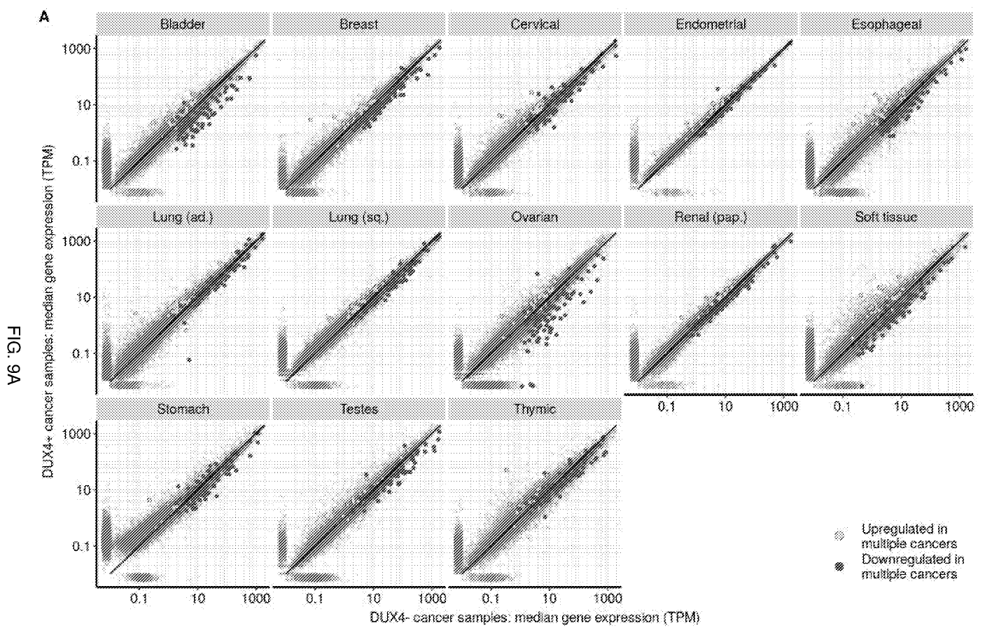

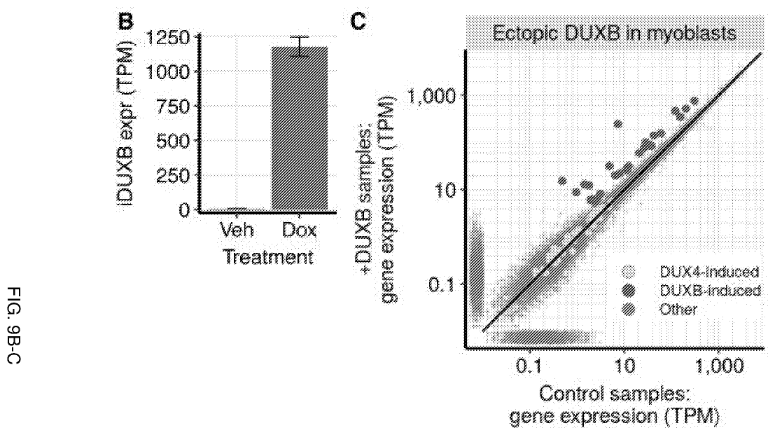

The inventors next tested whether DUX4 was expressed in solid cancers as a full-length DUX4 mRNA, or instead as a truncated DUX4 mRNA consisting of the 5′ end of the DUX4 mRNA fused to another gene product (as occurs in B-ALL). The inventors aligned reads from each DUX4+ solid cancer, preimplantation embryos, and B-ALL with DUX4 translocations to the full-length DUX4 mRNA sequence. Read coverage extended across the full-length DUX4 mRNA in preimplantation embryos, as expected, as well as in all DUX4+ solid cancers (FIG. 2A-C). In contrast, reads aligned only to the 5′-most half of the DUX4 mRNA in B-ALL, consistent with the known presence of DUX4 translocations in these leukemias (FIG. 2D). The inventors did not observe read coverage patterns consistent with expression of a fusion gene encoding an N- or C-terminally truncated fragment of DUX4 in any DUX4+ solid cancer (FIG. 8A-B).

Finally, the inventors tested whether mis-alignment of reads from DUX4C to DUX4 was a confounding factor in their analysis. The DUX4C and DUX4 open reading frames are highly similar, with the exception of 32 residues at DUX4C's C terminus. Identifying reads that mapped uniquely to the distinguishing regions of DUX4C and DUX4 revealed that while a few DUX4+ samples also exhibited detectable DUX4C expression, the vast majority of mapping reads aligned uniquely to DUX4 (FIG. 8C). Together with the inventors' analyses of DUX4 splicing and read coverage patterns, these data indicate that DUX4+ solid cancers express full-length DUX4 mRNA.

4. Genetic Variation and Somatic Mutations Contribute to DUX4 Expression in Cancer

Normally silenced in somatic cells, DUX4 becomes inappropriately re-expressed in the skeletal muscle of individuals with facioscapulohumeral muscular dystrophy (FSHD) due to cis- and/or trans-acting genetic variation that disrupts normal epigenetic repression of the DUX4 locus (Lemmers et al., 2012; 2010). The inventors hypothesized that similar mechanisms might contribute to DUX4 expression in cancers. The inventors first used reads mapping to the 3′ end of the DUX4 mRNA to assess whether the DUX4 mRNA was expressed from a “permissive” 4qA161 allele or a “non-permissive” (10qA or 4qB) allele, which respectively do or do not contain consensus polyadenylation sites that permit stable DUX4 mRNA expression (Lemmers et al., 2010; 2007). The inventors observed significantly more reads arising from permissive alleles (FIG. 2E; p<0.001), indicating that inherited genetic variation contributes to DUX4 expression in cancer as well as FSHD.

The inventors next identified trans-acting regulators of DUX4 expression in cancer. Two recent studies reported that DPPA2 and DPPA4 activate expression of Dux, a murine double homeobox gene that is expressed during early embryogenesis like DUX4 (De Iaco et al., 2019; Eckersley-Maslin et al., 2019). Although mice lack DUX4 and the relationship between human DPPA2 and DPPA4 and DUX4 has not been tested, the inventors wondered whether the same occurred in cancers. High DPPA2 and DPPA4 expression was strongly associated with DUX4 expression in testicular germ cell tumors but no other cancer types, suggesting that human DPPA2 and DPPA4 may activate DUX4 expression in some cell types (FIG. 2F). The inventors next tested whether somatic mutations affecting known repressors of the DUX4 locus were associated with DUX4 expression. The inventors identified all cancer samples with or without predicted loss-of-function mutations in 23 genes encoding validated or likely repressors of DUX4, including proteins encoded by Modifier of murine metastable epiallele (Momme) genes (Daxinger et al., 2013), components of the Nucleosome Remodeling Deacetylase and Chromatin Assembly Factor 1 complexes (Campbell et al., 2018), and other epigenetic factors (Haynes et al., 2018; Huichalaf et al., 2014; Ottaviani et al., 2009; Zeng et al., 2009). The inventors tested whether samples with loss-of-function mutations in each gene exhibited elevated DUX4 expression relative to wild-type samples for each cancer type. Mutations in 12 tested genes were significantly associated with increased DUX4 expression in one or more cancer types (FIG. 2G, 8D), suggesting that loss of epigenetic repressors of the DUX4 locus contributes to DUX4 re-expression in cancer.

One of the strongest signals in the inventors' mutational analysis arose from PRPF8, which encodes a core spliceosomal protein. Although PRPF8 is not a known repressor of DUX4, the inventors included PRPF8 in their analysis because the inventors previously found that PRPF8 physically associates with the DUX4 locus (Campbell et al., 2018). The inventors therefore experimentally tested whether PRPF8 inhibition induced DUX4 expression. The inventors knocked down (KD) PRPF8 in myoblasts isolated from a healthy individual and an individual whose FSHD was caused by cis-acting genetic variation that potentiated DUX4 de-repression. PRPF8 KD resulted in increased DUX4 expression and up-regulation of the DUX4 targets ZSCAN4 and TRIM43, confirming the presence of transcriptionally active DUX4 protein following PRPF8 KD (FIG. 2H). The extent of up-regulation was much higher in FSHD cells, as expected. In addition to identifying PRPF8 as a repressor of DUX4 expression, the inventors' results demonstrate that both cis-acting genetic variation and somatic mutations in trans-acting repressors contribute to DUX4 expression in cancer.

5. DUX4 Drives an Early Embryonic Gene Expression Program in Cancer

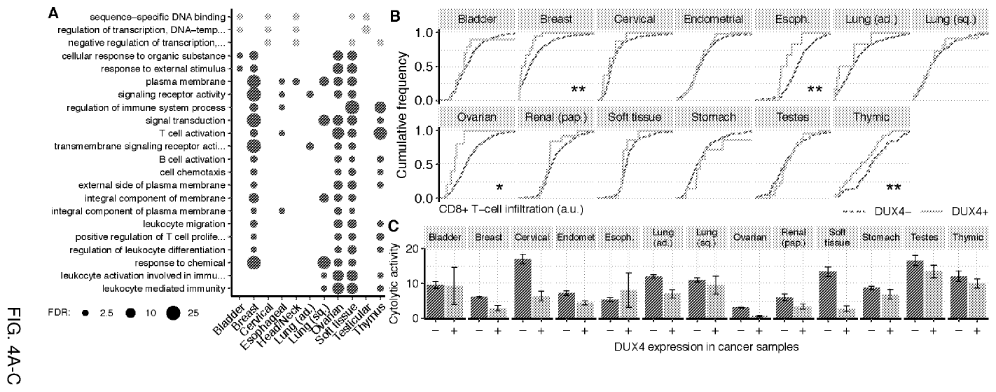

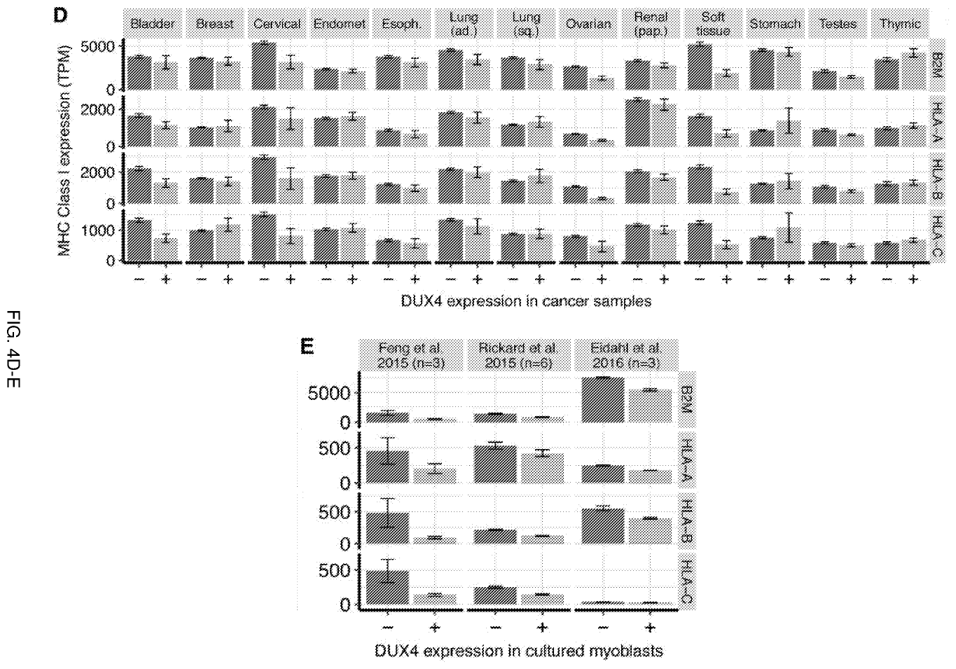

The inventors next tested whether DUX4 mRNA was likely translated into a functional transcription factor in DUX4+ cancers. As a pioneer transcription factor, DUX4 induces a stereotyped cleavage-stage gene expression program, even when it is aberrantly expressed in somatic cells outside of its normal embryonic context (FIG. 3A). DUX4 also binds to and transcriptionally activates specific repetitive elements (Geng et al., 2012; Young et al., 2013), many of which characterize the cleavage-stage gene expression program (Hendrickson et al., 2017). The inventors computed a high-confidence set of DUX4-induced target genes by intersecting the sets of genes associated with endogenous DUX4 expression during preimplantation embryogenesis as well as ectopic DUX4 expression in induced pluripotent stem cells (iPSCs) and myoblasts (Feng et al., 2015; Hendrickson et al., 2017). The inventors additionally defined a compact set of DUX4-responsive repetitive elements that were induced irrespective of cell type (Table S3). The inventors measured expression levels of each of these DUX4-induced genes and repetitive elements across each cancer cohort and compared their average expression in DUX4+ versus DUX4− cancers.

DUX4 expression was strongly associated with increased expression of DUX4 targets, including coding genes, non-coding genes, and repetitive elements (FIG. 3B). Several trends were notable. First, while DUX4+ cancers exhibited increased expression of many DUX4-induced genes irrespective of cancer type, one or more specific target genes were particularly highly expressed in each cancer type. For example, almost all DUX4+ thymomas expressed high levels of the DUX4 target CCNA1, while few bladder or breast cancers did. Second, the quantitative level of DUX4 target induction was highly variable across DUX4+ cancer samples, and was only modestly correlated with DUX4 expression levels. Third, most DUX4 targets were strongly induced in almost all DUX4+ testicular germ cell cancers. Testicular germ cell cancers might constitute unusually permissive environments for DUX4 activity, perhaps because DUX4 is endogenously expressed in luminal cells in the testis, most likely in the germline (Snider et al., 2010). The inventors conclude that DUX4 drives an early embryonic transcriptional program in diverse solid cancers.

6. DUXB is not Essential for the Core DUX4 Transcriptional Program in Cancer

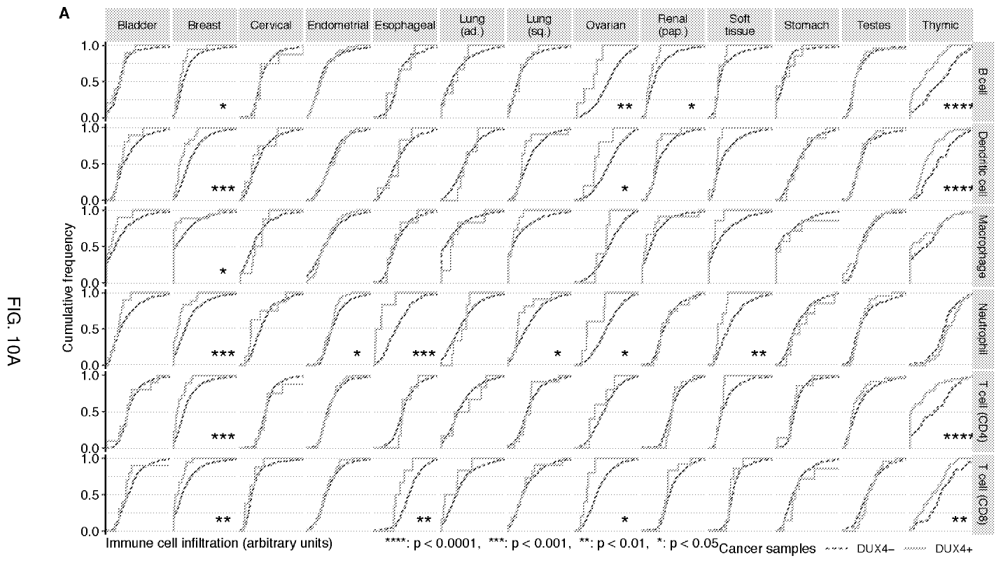

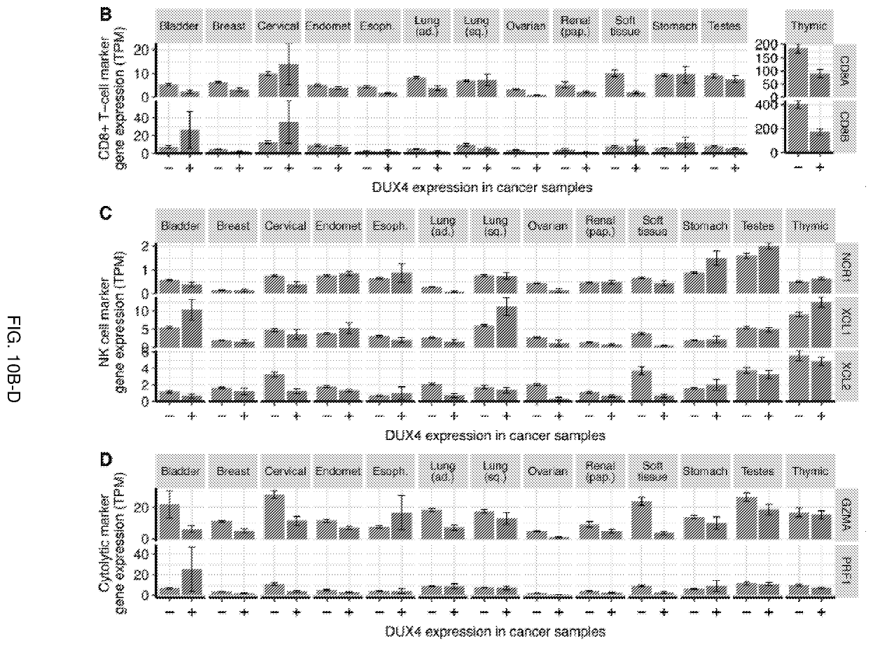

A recent study reported that Duxbl was recurrently amplified in a murine model of rhabdomyosarcoma and that its human ortholog DUXB was expressed in many human cancers (Preussner et al., 2018). Although the inventors also observed frequent DUXB expression in cancers, DUXB did not rank highly on their cancer specificity index because it is expressed in many healthy tissues (FIG. 1B). Nonetheless, as DUXB expression is promoted by DUX4 (FIG. 3A-B), the inventors wondered whether DUXB might contribute to the DUX4-induced gene expression program. The inventors established a myoblast cell line with a doxycycline-inducible DUXB transgene and performed RNA-seq on cells with or without doxycycline treatment. Although DUXB lacks DUX4's C-terminal transcriptional activation domain, DUXB induction resulted in statistically significant up-regulation of a small set of genes. However, DUXB had no effects on the inventors' high-confidence set of DUX4 targets (FIG. 9B-C, Table S4), suggesting that it is not essential for the DUX4-driven embryonic expression program in cancer.

7. DUX4 is Associated with Reduced Anti-Tumor Immune Activity

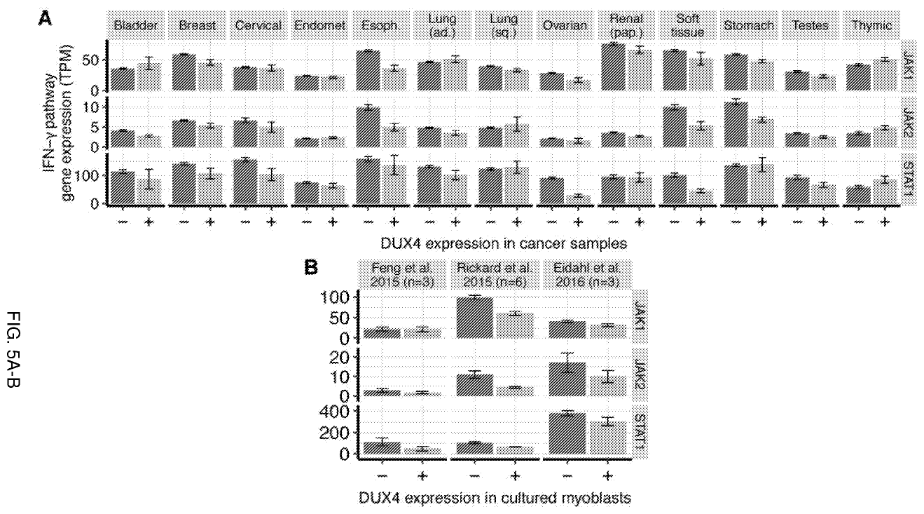

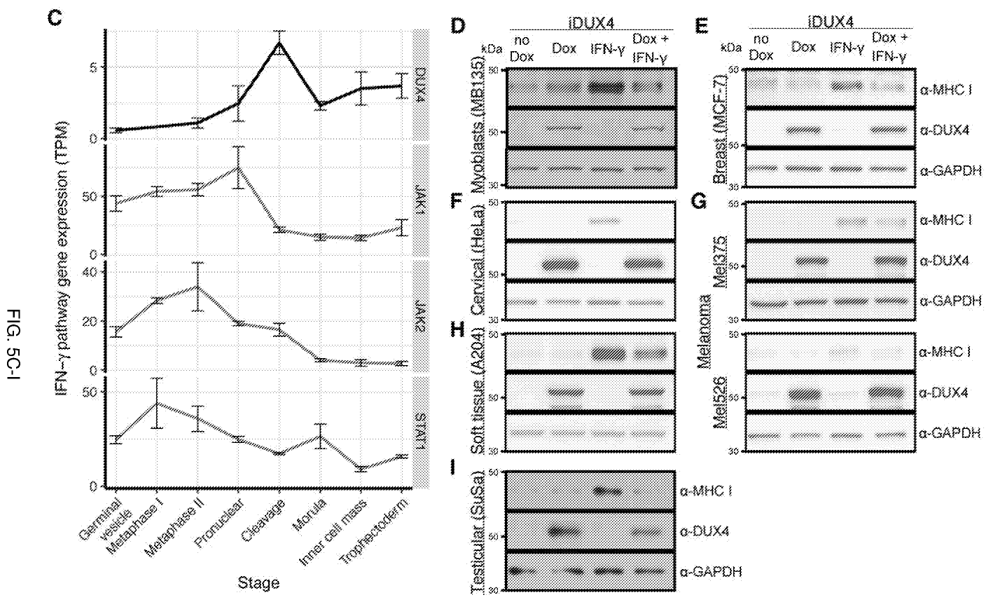

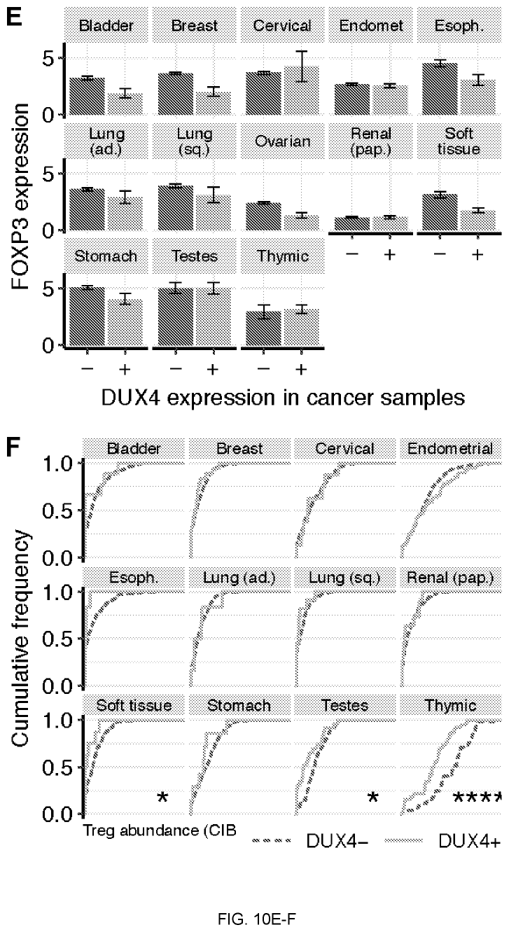

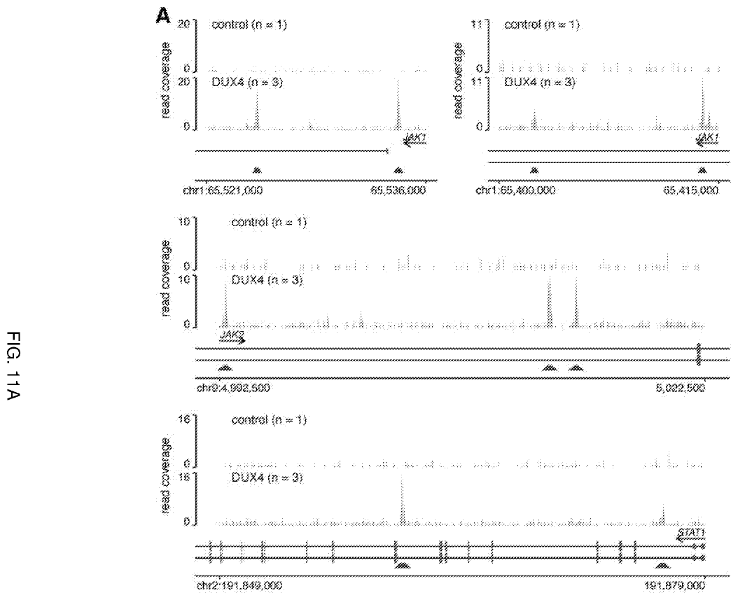

As many DUX4 targets encode CT antigens (Chang et al., 2011; Ikeda et al., 1997), the inventors wondered whether DUX4 re-expression might promote anti-tumor immune activity. The inventors therefore tested whether DUX4-expressing cancers exhibited gene expression signatures of high immune infiltration. To the inventors' surprise, multiple lines of evidence indicated that DUX4 expression was associated with decreased, rather than increased, anti-tumor immune activity. First, the inventors identified genes that were consistently differentially expressed in multiple cancer types in DUX4+ versus DUX4− samples and performed a Gene Ontology (GO)-based analysis of enriched functional categories (FIG. 9A). Almost all enriched GO terms belonged to immune-related categories. However, contrary to the inventors' hypothesis that DUX4 expression might trigger immune surveillance, the immune-related GO terms were uniformly associated with decreased gene expression in DUX4+ versus DUX4− cancers (FIG. 4A). Second, the inventors noticed that many immune cell-specific genes were downregulated in DUX4+ cancers, suggesting that reduced immune cell infiltration might underlie this GO enrichment signature. The inventors therefore estimated infiltration of different immune cell types in DUX4+ and DUX4− cancers with the TIMER algorithm (Li et al., 2017). DUX4 expression was associated with reduced infiltration of diverse immune cells, most notably cytotoxic CD8+ T cells, in many cancers (FIG. 4B, 10A-B). Natural killer (NK) cell-specific markers exhibited similarly reduced expression in many DUX4+ cancer types (FIG. 10C). Third, the inventors estimated anti-tumor immune activity by measuring the expression of GZMA and PRF1, which encode two key cytolytic factors expressed by cytotoxic T cells and NK cells (Rooney et al., 2015), to find that cytolytic activity was markedly lower in most DUX4+ versus DUX4− cancers (FIG. 4C, 10D).

As immunosuppressive regulatory T cells (Tregs) play important roles in tolerance of self-antigens (Sakaguchi et al., 2008), the inventors wondered whether DUX4 might create an immunosuppressive environment by altering Treg recruitment. The Treg marker gene FOXP3 was significantly down-regulated in DUX4+ samples in a few, although not most, cancer types (FIG. 10E). However, this association did not persist when the inventors estimated Treg infiltration with the more sophisticated CIBERSORT algorithm (Newman et al., 2015; Thorsson et al., 2018) (FIG. 10F), suggesting that DUX4-mediated immunosuppression is likely not explained by Treg recruitment.

8. DUX4 Suppresses MHC Class I Expression

As DUX4 promotes CT antigen expression yet DUX4+ cancers exhibited low anti-tumor immune activity, the inventors wondered whether antigen presentation might be suppressed in DUX4-expressing cells. Reduced expression or loss of the HLA (Human Leukocyte Antigen) or B2M (132 microglobulin) genes, which encode MHC Class I molecules that display peptides for immune recognition, is a common mechanism by which cancers evade immune surveillance (Shukla et al., 2015). The inventors therefore compared the expression levels of MHC Class I genes in DUX4+ and DUX4− cancers to find that DUX4 expression was associated with reduced expression of B2M, HLA-A, HLA-B, and HLA-C in most cancer types (FIG. 4D).

Decreased expression of MHC Class I genes could be a direct consequence of DUX4 expression, or alternatively might be an independent event that enhances the survival of DUX4-expressing cancers. For example, cancers that evade immune surveillance might be under reduced immune selection, enabling them to subsequently express DUX4 and its antigenic targets without deleterious consequences. To distinguish between those possibilities, the inventors studied acute DUX4 expression in cell culture. The inventors re-analyzed two RNA-seq datasets in which DUX4 was ectopically expressed in DUX4− cells (Eidahl et al., 2016; Feng et al., 2015) and one dataset in which differentiating myoblasts that spontaneously expressed DUX4 were flow-sorted into DUX4+ and DUX4− pools (Rickard et al., 2015). The inventors found that DUX4-expressing cells exhibited reduced levels of MHC Class I genes relative to DUX4− control cells in all three datasets (FIG. 4E). As immunoediting is not a potential confounding factor in these cell culture experiments, The inventors conclude that DUX4 is a cell-intrinsic suppressor of MHC Class I.

9. DUX4 Suppresses Interferon-γ-Mediated Induction of MHC Class I-Dependent Antigen Presentation

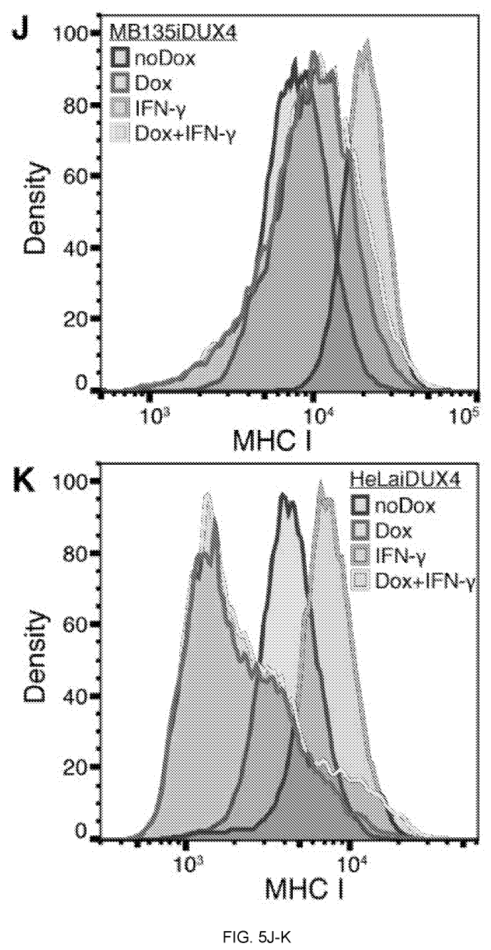

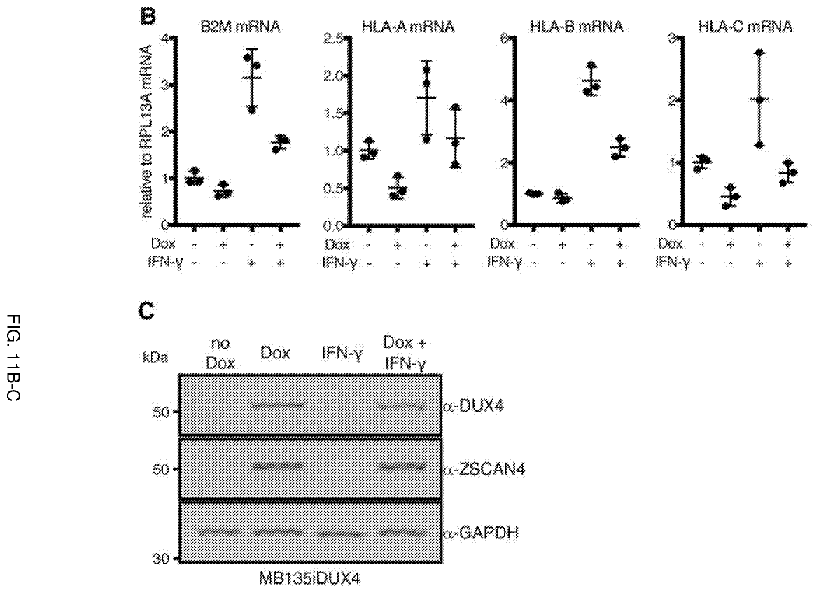

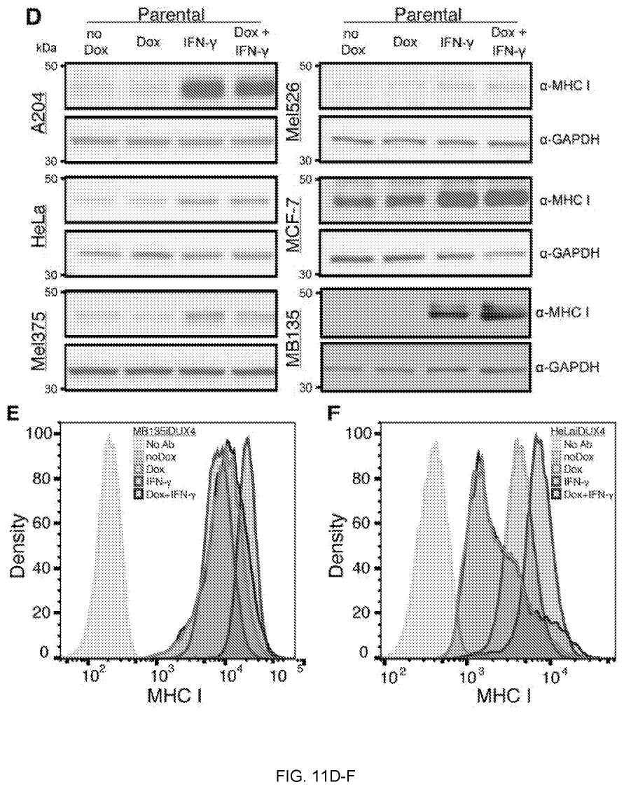

Tumor-infiltrating immune cells induce antigen presentation on malignant cells by secreting interferon-γ, resulting in up-regulation of MHC Class I genes via JAK1, JAK2, and STAT1-dependent signal transduction (Friedman et al., 1984; Schroder et al., 2004). The inventors noted that DUX4 expression was associated with reduced expression of JAK1, JAK2, and/or STAT1 in many cancer types (FIG. 5A). JAK1, JAK2, and STAT1 exhibited similarly reduced expression following acute DUX4 expression in cultured myoblasts (FIG. 5B) as well as after the onset of endogenous DUX4 expression in preimplantation embryos (FIG. 5C). Finally, the inventors noted that JAK1, JAK2, and STAT1 each exhibited multiple peaks of DUX4 binding in their published ChIP-seq dataset of acute DUX4 expression in cultured myoblasts (FIG. 11A) (Geng et al., 2012).

The inventors therefore tested whether DUX4 could block interferon-γ-mediated induction of MHC Class I. The inventors initially used MB135iDUX4 cells, which permit doxycycline-inducible DUX4 expression in a myoblast cell line. MB135iDUX4 cells are a validated model of the biological consequences of DUX4 expression that recapitulate the cleavage-stage transcriptional program (Hendrickson et al., 2017; Jagannathan et al., 2016; Whiddon et al., 2017). Treatment with interferon-γ resulted in robust up-regulation of MHC Class I protein, as expected, while inducing DUX4 by adding doxycycline effectively blocked this up-regulation of MHC Class I (FIG. 5D). DUX4 induction drove expression of the DUX4-activated embryonic gene ZSCAN4 independent of interferon-γ treatment while suppressing interferon-γ-mediated up-regulation of B2M, HLA-A, HLA-B, and HLA-C mRNA (FIG. 11B-C). Adding doxycycline in the absence of the inducible DUX4 construct had no effect on interferon-γ-mediated induction of MHC Class I (FIG. 11D).

The inventors next tested whether DUX4 blocked interferon-γ-mediated induction of MHC Class I protein in cancer cells. The inventors introduced a doxycycline-inducible DUX4 construct into six cell lines derived from five cancer types: breast adenocarcinoma (MCF-7), cervical cancer (HeLa), melanoma (Me1375 and Me1526), rhabdomyosarcoma (A204), and testicular teratocarcinoma (SuSa). Treatment with interferon-γ induced higher levels of MHC Class I protein in all tested cell lines, and DUX4 suppressed this induction (FIG. 5E-I). Treating the parental cell lines with doxycycline had no effect on interferon-γ-mediated induction of MHC Class I (FIG. 11D).

Finally, the inventors confirmed that DUX4 blocked interferon-γ-mediated induction of MHC Class I-dependent antigen presentation. As peptide binding is required for MHC Class I stability and cell surface localization (Townsend et al., 1989), the inventors used flow cytometry to measure how DUX4 affected levels of MHC Class I on the cell surfaces of representative untransformed (MB135) and cancer (HeLa) cell lines. Interferon-γ treatment drove robust up-regulation of MHC Class I on the cell surface, as expected, while DUX4 abrogated this induction in both cell types (FIG. 5J-K, 11E-F). DUX4-mediated suppression of MHC Class I was particularly notable in HeLa cells, where DUX4 suppressed cell surface levels of MHC Class I beyond the basal state even in the presence of interferon-γ. Together, these data demonstrate that DUX4 blocks interferon-γ-mediated induction of MHC Class I and antigen presentation in untransformed and cancer cells.

10. DUX4 Promotes Resistance to Immune Checkpoint Blockade

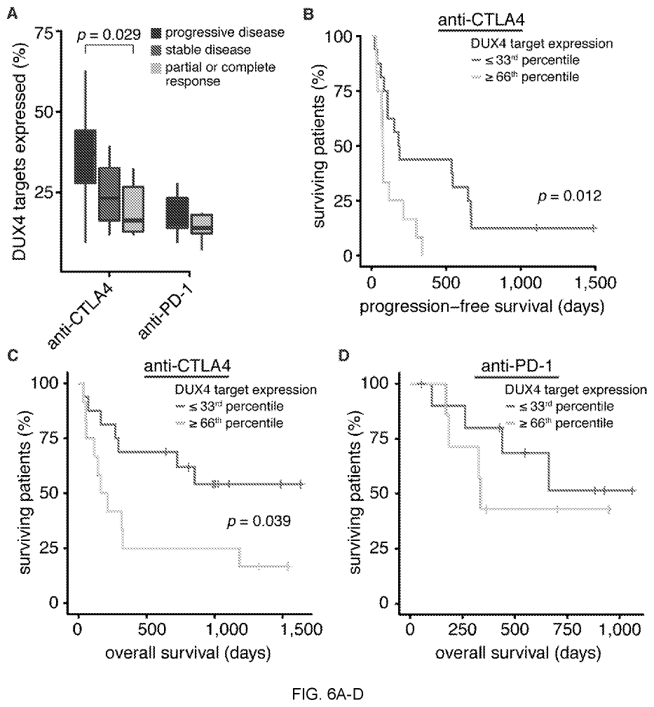

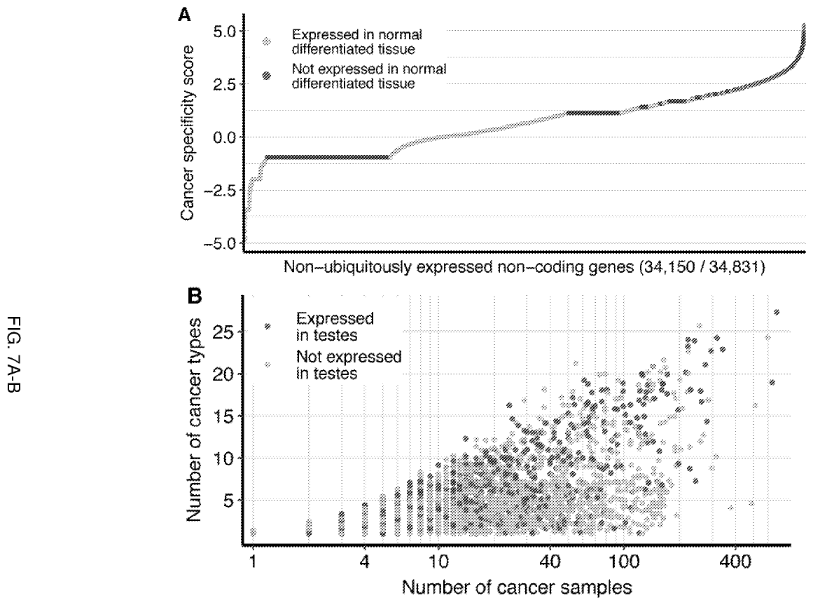

As immune checkpoint blockade relies upon antigen presentation, resistance to these therapies is strongly associated with loss of antigen presentation as well as loss of interferon-γ signal transduction (Gao et al., 2016; Sade-Feldman et al., 2017; Zaretsky et al., 2016). The inventors therefore hypothesized that DUX4-mediated suppression of antigen presentation might promote resistance to checkpoint blockade. The inventors analyzed RNA-seq data from pretreatment biopsies across two cohorts in which patients with metastatic melanoma were treated with anti-CTLA-4 (Van Allen et al., 2015) or anti-PD-1 (Hugo et al., 2016). Biopsies from patients whose disease was clinically classified as non-responsive to anti-CTLA-4 exhibited significantly higher levels of DUX4 transcriptional activity relative to biopsies from responsive patients (FIG. 6A). Stratifying patients according to DUX4 transcriptional activity revealed dramatic and statistically significant differences in both progression-free and overall survival following anti-CTLA-4 therapy (FIG. 6B-C). Increased DUX4 transcriptional activity was similarly associated with failure to respond to anti-PD-1 therapy (FIG. 6A) and decreased overall survival following anti-PD-1 treatment (FIG. 6D), although the differences were not statistically significant, perhaps because of the smaller size of the anti-PD-1 cohort (27 versus 41 patients) or a more predictive role for MHC Class I expression in response to anti-CTLA-4 than anti-PD-1 therapy (Rodig et al., 2018). The inventors conclude that DUX4-mediated suppression of MHC Class I-dependent antigen presentation is a clinically relevant biomarker for response to immune checkpoint blockade.

C. Discussion

The inventors' observation that DUX4 activity is significantly associated with failure to respond to immune checkpoint blockade (FIG. 6) provides a clinical motivation for determining when, where, and how DUX4 becomes re-expressed in cancers. Future prospective studies of larger cohorts are essential to confirm the inventors' results and test the clinical utility of using DUX4 activity as a predictive biomarker of response to checkpoint blockade.

DUX4 is aberrantly expressed in both FSHD muscle and cancers, but the physiological consequences of DUX4 expression in these two disease states are quite different. Sustained expression of DUX4 in skeletal muscle causes apoptosis (Eidahl et al., 2016; Kowaljow et al., 2007), in contrast to DUX4's importance during early embryogenesis and apparent compatibility with many malignancies. Determining why early embryos and cancer cells can tolerate DUX4 expression, while muscle cells cannot, may give insight into possible mechanisms for treating FSHD. Another notable difference is the frequent presence of inflammation and lymphocytic infiltration in FSHD muscle (Arahata et al., 1995) versus reduced immune infiltration in DUX4+ cancers. As DUX4 suppresses MHC Class I in both untransformed muscle cells and cancer cells (FIG. 5D-I, 11B-D), further work is required to determine why DUX4 expression results in immune attack in FSHD muscle but immune evasion in cancers.

The inventors' finding of full-length DUX4 expression and transcriptional activity in diverse solid cancers is mechanistically distinct from the prior identification of DUX4 translocations in other cancer types. All DUX4-IGH fusion proteins lack DUX4's C-terminal activation domain and do not activate DUX4 target gene expression (Lilljebjorn et al., 2016; Liu et al., 2016; Tanaka et al., 2018; Yasuda et al., 2016; Zhang et al., 2016). The CIC-DUX4 fusion protein combines CIC's high mobility group-box DNA-binding domain with the transcriptional activation domain of DUX4, and so dysregulates CIC target genes but should not activate DUX4 target gene expression (Specht et al., 2014). While the DUX4-IGH and CIC-DUX4 fusion proteins likely possess oncogenic capacities, neither activates the gene expression program that is characteristic of totipotent embryonic cells expressing full-length DUX4.

Since DUX4 re-expression is common in many cancers, why has it not been previously detected? First, DUX4 is a multicopy gene that lies within the D4Z4 macrosatellite repeat array in the subtelomeric region of chromosome 4q (Gabriëls et al., 1999; Lee et al., 1995). The repetitive nature of DUX4's genomic locus and its highly variable copy number in the human population (Wijmenga et al., 1993) render it difficult to study, possibly hindering its prior identification as a cancer-specific gene. The continued development of experimental and computational techniques for querying repetitive genomic loci may facilitate the identification of additional genes like DUX4 that play unexpected roles in cancer. Second, the DUX4 mRNA is rapidly turned over by nonsense-mediated decay (Feng et al., 2015) and is present at relatively low abundance in its normal developmental context as well as in cancers. Third, because the cleavage-stage embryonic transcriptome that DUX4 activates was only recently characterized (De Iaco et al., 2017; Hendrickson et al., 2017; Whiddon et al., 2017), it would not have been revealed by Gene Ontology-like enrichment analyses of cancer-expressed genes. For all of these reasons, quantifying expression of the genes that the inventors identified as robust DUX4 targets in both embryos and cancers (Table S3) may prove to be an efficient method for identifying DUX4-expressing cancers.

The inventors' data implicate DUX4 in MHC Class I-dependent antigen presentation, but do not exclude the possibility that DUX4 regulates tumor-immune interactions via other mechanisms as well. For example, DUX4 could influence T cell exclusion from the tumor microenvironment. The inventors noted that many DUX4+ cancers exhibited reduced levels of the chemoattractants CXCL9 and CXCL10 (FIG. 11G), which promote lymphocyte recruitment to tumors (Homey et al., 2002). The inventors experimentally confirmed that DUX4 prevented interferon-γ-stimulated increases in CXCL9 and CXCL10 mRNA in myoblasts (FIG. 11H), suggesting that DUX4 may influence chemokine signaling in addition to altering antigen presentation. Further work is required to confirm that DUX4 influences CXCL9 and CXCL10 levels in cancer cells and identify all of the potentially diverse means by which DUX4 contributes to immune evasion.

In addition to facilitating immune evasion, DUX4 might promote tumorigenesis through additional mechanisms that regulate normal early embryonic development. For example, the DUX4 target ZSCAN4 is required for telomere maintenance and extension in embryonic stem cells (Zalzman et al., 2010). ZSCAN4 is activated in most DUX4+ solid cancers, where it may similarly contribute to the replicative potential of these cancers. Another example is the DUX4 target CCNA1, which encodes an A-type cyclin that is essential for male meiosis (Liu et al., 1998) and aberrantly expressed in many myeloid malignancies (Kramer et al., 1998). Ectopic expression of CCNA1 in the murine hematopoietic lineage caused abnormal myelopoiesis and sporadic progression to acute myeloid leukemia (Liao et al., 2001). These are just two examples illustrating how DUX4 targets' normal roles in the totipotent cleavage-stage embryo and germ cells may contribute to tumorigenesis. While the functional roles of many DUX4 target genes (Table S3) are undefined, the inventors hypothesize that other DUX4 targets may directly contribute to cancer initiation and progression.

As others have long noted, preimplantation embryos bear many qualitative similarities to malignant cells, including de-/re-programming of cell state, infinite replicative potential, and the capacity to effectively invade other tissues (Ben-Porath et al., 2008; Hanahan and Weinberg, 2000; Monk and Holding, 2001; Pierce, 1983). As DUX4 activates the transcriptional program defining the cleavage stage of early embryogenesis, DUX4 targets and downstream factors presumably enable preimplantation embryos to acquire these cancer-like characteristics. DUX4's ability to suppress MHC Class I-dependent antigen presentation provides a mechanistic connection between preimplantation development and immune evasion, which is now widely recognized as a hallmark of cancer (Hanahan and Weinberg, 2011). The inventors' discovery that diverse cancers express transcriptionally active DUX4 suggests a model whereby re-expression of an embryonic transcription factor activates an early developmental program characteristic of totipotent cells, resulting in the transformation of somatic cells into malignancies that can evade immune destruction.

D. Experimental Model and Subject Details

1. Cell lines.

A204 cells were purchased from ATCC (Cat #HTB-82) and have been cultured in the Tapscott laboratory since 2012. HeLa cells were purchased from ATCC (Cat #CCL-2) and have been cultured long-term in the Tapscott laboratory. All myoblast lines were obtained as de-identified primary cells from the Fields Center for FSHD and Neuromuscular Research at the University of Rochester Medical Center (available on the world wide web at urmc.rochester.edu/neurology/fields-center.aspx) and immortalized by retroviral transduction of CDK4 and hTERT (Stadler et al., 2011) in the Tapscott laboratory. MB135iDUX4 cells have also been described previously (Jagannathan et al., 2016). MCF-7 cells were purchased from ATCC (Cat #HTB-22) and have been cultured in the Tapscott laboratory since 2017. Me1375 and Me1526 cells were generously provided by Dr. Seth Pollack (Pollack et al., 2012). SuSa cells were purchased from DSMZ (Cat #ACC 747) and have been cultured in the Tapscott laboratory since 2014.

2. Cell Culture.

A204 and A204iDUX4 cells were maintained in RPMI 1640 Medium (Gibco) supplemented with 10% HyClone Fetal Bovine Serum (GE Healthcare Life Sciences), 100 U/100 μg penicillin/streptomycin (Gibco), and, for A204iDUX4, 1.5 μg/ml puromycin (Sigma-Aldrich). HeLa and HeLaiDUX4 cells were maintained in Dulbecco's Modified Eagle Medium (Gibco) supplemented with 10% HyClone Fetal Bovine Serum, 100 U/100 μg penicillin/streptomycin, and, for HeLaiDUX4, 1.5 μg/ml puromycin. MB2401, MB073, MB135, MB135iDUX4, and MB135iDUXB myoblasts were maintained in Ham's F-10 Nutrient Mix (Gibco) supplemented with 20% HyClone Fetal Bovine Serum, 100 U/100 μg penicillin/streptomycin (Gibco), 10 ng/ml recombinant human basic fibroblast growth factor (Promega Corporation), 1 μM dexamethasone (Sigma-Aldrich), and, for MB135iDUX4 and MB135iDUXB, 3 μg/ml or 2 μg/ml puromycin (Sigma-Aldrich), respectively. MCF-7 and MCF-7iDUX4 cells were maintained in Eagle's Minimal Essential Medium (Quality Biological) supplemented with 10% HyClone Fetal Bovine Serum, 100 U/100 μg penicillin/streptomycin, 10 μg/ml insulin (Sigma-Aldrich), and, for MCF7iDUX4, 1.5 μg/ml puromycin. Me1375, Me1375iDUX4, Me1526, and Me1526iDUX4 cells were maintained in RPMI 1640 Medium containing HEPES (Gibco) supplemented with 10% HyClone Fetal Bovine Serum, 100 U/100 μg penicillin/streptomycin, 2 mM L-Glutamine (Gibco), 1% MEM Non-Essential Amino Acids Solution (Gibco), 1 mM Sodium Pyruvate (Gibco), and, for Me1375iDUX4 and Me1526iDUX4, 2 μg/ml or 1 μg/ml puromycin, respectively. SuSa and SuSaiDUX4 cells were maintained in RPMI 1640 Medium supplemented with 10% HyClone Fetal Bovine Serum, 100 U/100 μg penicillin/streptomycin, and, for SuSaiDUX4, 1 μg/ml puromycin. A204iDUX4, HeLaiDUX4, MCF-7iDUX4, Me1375iDUX4, Me1526iDUX4, SuSaiDUX4, and MB135iDUXB cell lines were generated as previously described for MB135iDUX4 cells (Jagannathan et al., 2016).

E. Method Details

1. Data Sources.

RNA-seq reads were downloaded from CGHub (TCGA), the Gene Expression Omnibus (accession numbers GSE85632, GSE85935, GSE78220) (Eidahl et al., 2016; Hendrickson et al., 2017; Hugo et al., 2016), the NCBI sequence read archive (SRA) database (accession number SRP058319) (Rickard et al., 2015), dbGaP (accession number phs000452.v2.p1) (Van Allen et al., 2015), the Japanese Genotype-Phenotype Archive (accession number JGAS00000000047) (Yasuda et al., 2016), and the European Genome-phenome Archive (accession number EGAD00001002112) (Lilljebjorn et al., 2016). Sample annotations and gene expression data were downloaded from the GTEx portal (www.gtexportal.org). ChIP-seq reads were downloaded from the Gene Expression Omnibus (accession number GSE33838) (Geng et al., 2012).

2. RNA-Seq Library Preparation.

Total RNA integrity was checked using a 4200 TapeStation System (Agilent Technologies) and quantified using a Trinean DropSense 96 UV-Vis spectrophotometer (Caliper Life Sciences). The RNA-seq libraries were prepared from total RNA using the TruSeq RNA Sample Prep Kit v2 (Illumina). Library size distribution was validated using a 4200 TapeStation System. Additional library quality control, blending of pooled indexed libraries, and cluster optimization was performed using a Qubit 2.0 Fluorometer (Life Technologies-Invitrogen). RNA-seq libraries were pooled (16-plex) and clustered onto 2 flow cell lanes. Sequencing was performed using an Illumina HiSeq 2500 in high-output mode employing a single-end, 100 base read length sequencing strategy. All work was carried out by the Fred Hutch Genomics Shared Resource.

3. Genome Annotation, RNA-Seq and ChIP-Seq Read Mapping, and Gene Expression Estimation.

A genome annotation was created by merging the UCSC knownGene (Meyer et al., 2013), Ensembl 71 (Flicek et al., 2013), and MISO v2.0 (Katz et al., 2010) annotations. RNA-seq reads were mapped to this annotation as previously described (Dvinge et al., 2014). In brief, RSEM v1.2.4 (Li and Dewey, 2011) was modified to call Bowtie v1.0.0 (Langmead et al., 2009) with the option ‘-v 2’ and then used to map all reads to the merged genome annotation. Remaining unaligned reads were then mapped to the genome (GRCh37/hg19 assembly) and a database of potential splice junctions with TopHat v2.0.8b (Trapnell et al., 2009). All gene expression estimates were normalized using the trimmed mean of M values (TMM) method (Robinson and Oshlack, 2010). For GTEx data, per-tissue gene expression estimates were obtained by computing the median expression over all samples for a given tissue following TMM normalization. Read alignments to the full-length DUX4 cDNA were performed with Kallisto v0.43.0's pseudoalignment function (Bray et al., 2016). Subsequent visualization of the resulting read coverage was performed with IGV (Thorvaldsdottir et al., 2013). ChIP-seq reads were mapped to the genome with Bowtie v1.0.0 (Langmead et al., 2009) with the arguments ‘-v 2-k 1-m 1—best—strata’. ChIP-seq peaks were called with MACS v 2.1.1.20160309 (Zhang et al., 2008) with the arguments ‘callpeak-g hs’.

4. Cancer-Specific Expression Score.

The cancer-specific expression score of each gene was defined as the logarithm of the fractions of cancer samples and types in which the gene was expressed divided by the fractions of peritumoral samples and normal tissues in which the gene was expressed. The inventors used the following thresholds to define a gene as expressed or not expression in a given sample. Following TMM normalization, each gene within each sample was classified as expressed (>1.5 TPM), not expressed (<0.5 TPM in normal tissue), or uncertain (<1.5 TPM, but >0.5 TPM). For peritumoral samples, the inventors relaxed the threshold for defining genes as not expressed to <1.0 TPM in order to allow for potential cancer field effects, sample contamination, or other confounding factors. The inventors defined DUX4+ and DUX4− cancer samples as those with DUX4 expression >1.5 TPM or <0.5 TPM.

5. DUX4 Polyadenylation Site Usage.

The alleles from which DUX4 mRNAs were expressed in a sample were determined from diagnostic polymorphisms in exon 2 of DUX4 (Snider et al., 2010). This information was inferred from the nucleotide sequences at genomic positions chrUn_g1000228:114,025-114,056 in mapped RNA-seq reads, corresponding to the sequences shown in Table 4 of Snider et al., 2010. Mapped RNA-seq reads containing the DUX4 exon 3 polyadenylation sites were defined as reads that overlapped the ATATATTAAA sequence at chrUn_g1000228:114,642-114,651 with no mismatches.

6. Somatic Mutation Analysis.

TCGA somatic mutation calls from the Mutect pipeline (Cibulskis et al., 2013), together with their phenotypic impact as predicted by PolyPhen (Adzhubei et al., 2010) were obtained using the GDCquery_Maf function from TCGAbiolinks (Colaprico et al., 2016). Mutations were segregated as deleterious (frameshift, nonsense, and predicted deleterious mutations), low impact (silent and predicted tolerated) or other. In each cancer cohort, for each gene tested, samples with deleterious mutations were compared to samples with low impact or no mutations.

7. DUX4 and DUX4C Comparison.

RNA-seq reads that mapped uniquely to full-length DUX4 mRNA or DUX4C mRNA were extracted using samtools view (Li et al., 2009) with the following coordinates in the GRCh37/hg19 assembly: DUX4, chrUn_g1000228:113631-113879; DUX4C, chr4:190942696-190942795 and chrUn 1000228:26525-26624 (those two loci have identical genomic sequences). The density of reads was calculated as the total number of reads mapping to the region, normalized by the lengths of the regions queried.

8. DUX4 and DUX4-s Isoform Comparison

Uniquely identifying splice junctions for the long and short isoforms of DUX4 mRNA were identified by their 5′ splice sites at chrUn 1000228:113,887 and chrUn_g1000228:113,081, respectively.

9. DUXB Differential Gene Expression Analysis.

DUXB target genes were defined as those genes that were expressed at >5 TPM following DUXB induction, exhibited a fold-change >2 relative to uninduced samples, and had an associated Bayes factor of >10 as computed with Wagenmakers's framework (Wagenmakers et al., 2010) in both experimental replicates.

10. DUX4 Differential Gene Expression Analysis.

A high-confidence set of DUX4 target genes were defined as expressed at >5 TPM in DUX4+ samples, with a greater than 4-fold change over DUX4− samples, and with a Bayes factor of >10 as computed with Wagenmakers's framework (Wagenmakers et al., 2010) in all experimental replicates, across samples from pre-implantation embryos (Hendrickson et al., 2017), myoblasts (Feng et al., 2015) and iPSCs (Hendrickson et al., 2017).

11. Differential Gene Expression and Gene Ontology Analyses.

Genes that were differentially expressed in DUX4+ versus DUX4− cancer samples were determined with a two-sided Mann-Whitney test, as implemented in wilcox.test in R, with a p-value threshold of 0.01 and a fold-change threshold of 2.0. Gene Ontology (GO) terms that were enriched amongst genes that exhibited increased or decreased expression in DUX4+ versus DUX4− samples were identified with the GOseq method (Young et al., 2010), with a false-discovery rate threshold of 0.01. The intersection between the resulting significant GO terms that were identified for each cancer type was then computed, and only the child-most terms of the resulting intersection were analyzed further and reported.

12. Quantification of Immune Cell Infiltration.

Estimates of immune cell infiltration of TCGA primary tumors were downloaded from the TIMER web server (Li et al., 2017) and cross-referenced to the inventors' classification of each sample as DUX4+ or DUX4-. A one-sided Mann-Whitney U test was used to test for a statistically significant difference in immune cell infiltration between DUX4- and DUX4+ cancer samples.

13. Cloning

The DUXB gene was codon altered, synthesized by IDT custom gene synthesis, and subcloned by restriction enzyme digest into the Nhel and Sall sites of the pCW57.1 vector, a gift from David Root (Addgene plasmid #41393).

14. siRNA Transfection.

FlexiTube GeneSolution siRNAs targeting PRPF8 (Cat. no. GS10594) and a non-targeting Negative Control siRNA (Cat. no. 1022076) were obtained from Qiagen. Transfection of siRNAs into myoblasts was carried out as previously described (Campbell et al., 2018).

15. MHC Class I Immunoblotting and Induction by Interferon-γ.

Cells were treated with 1 μg/ml doxycycline hyclate (Sigma-Aldrich) for four hours and then stimulated with 100-200 ng/ml interferon-γ (R&D Systems) for 14-17 hours. Whole-cell protein extracts were obtained by lysing cells directly in 4×SDS sample buffer (500 mM Tris-HCl pH 6.8, 8% SDS, 20% 2-mercaptoethanol, 0.004% bromophenol blue, 30% glycerol) or RIPA buffer (150 mM NaCl, 1% Nonidet P-40, 0.5% sodium deoxycholate, 0.1% SDS, 25 mM Tris-HCl pH 7.4), followed by sonication. Protein extracts were run on NuPAGE 4-12% precast polyacrylamide gels (Invitrogen) and transferred to nitrocellulose membrane (Invitrogen). Membranes were blocked in PBS containing 0.1% Tween-20 and 5% non-fat dry milk before overnight incubation at 4° C. with primary antibodies. Membranes were then incubated with horseradish peroxidase-conjugated secondary antibodies in block solution for 1 hour at room temperature and chemiluminescent substrate (Thermo Fisher Scientific) was used for detection on film or with a ChemiDox MP Imaging System (Bio-Rad). Membranes were stripped with Restore Western Blot Stripping Buffer (Pierce) before being re-probed.

16. Real-Time qPCR

Total RNA was extracted from whole cells using the NucleoSpin RNA kit (Machery-Nagel) according to the manufacturer's instructions. Isolated RNA was treated with DNase I (Thermo Fisher Scientific), heat inactivated, and reverse transcribed into cDNA using SuperScript III (Thermo Fisher Scientific) and oligo(dT) primers (Invitrogen) following the manufacturer's protocol. Quantitative PCR was carried out on a QuantStudio 7 Flex (Applied Biosystems) using primers specific for each mRNA and iTaq SYBR Green Supermix (Bio-Rad Laboratories, Inc.). The relative expression levels of target genes were normalized to that of reference housekeeping genes by using the delta-delta-Ct method (Livak and Schmittgen, 2001) after confirming equivalent amplification efficiencies of reference and target molecules.

17. Flow Cytometry.

+Cells were treated with 1 ug/ml doxycycline hyclate (Sigma-Aldrich) for four hours and then stimulated with 200 ng/ml interferon-γ (R&D Systems) for 16 hours. Cells were harvested using trypsin, washed with FACS Buffer (1×DPBS, 2% FBS), and then stained with a 1:50 dilution of FITC-conjugated MHC Class I antibody (Biolegend) for 30 minutes at 4□C. Following staining, cells were washed again and resuspended in FACS buffer for analysis by flow cytometry using BD LSRFortessa X-50 with BD FACSDiva software (BD Biosciences). Data were analyzed using FlowJo v10.5.3.

18. Antibodies.

The following antibodies were used: GAPDH (6C5) (GeneTex, Inc. GTX28245; Lot #23184, Lot #821705388, or Lot #821803139), MHC Class I (F-3) (Santa Cruz Biotechnology sc-55582; Lot #C0817 or Lot #L1118), FITC anti-human HLA-A,B,C (W6/32) (BioLegend 311404; Lot #B223038), ZSCAN4 (Invitrogen PAS-32106; Lot #SK2479411A), Peroxidase AffiniPure Goat Anti-Mouse IgG (H+L) (Jackson ImmunoResearch 115-035-146), and Peroxidase AffiniPure Goat Anti-Rabbit (H+L) (Jackson ImmunoResearch 111-035-144), and a previously described rabbit monoclonal antibody against DUX4 (E14-3) that was produced in collaboration with Epitomics (Geng et al., 2011).

19. Primers

The following primers were used: B2M F: ACTGAATTCACCCCCACTGA (SEQ ID NO:72) (Zhang et al., 2005); B2M R: CCTCCATGATGCTGCTTACA (SEQ ID NO:73); CXCL9 F: TCTTTTCCTCTTGGGCATCA (SEQ ID NO:74) (Zeisel et al., 2013); CXCL9 R: TAGTCCCTTGGTTGGTGCTG (SEQ ID NO:75); CXCL10 F: GTGGCATTCAAGGAGTACCTC (SEQ ID NO:76) (Wang et al., 2012); CXCL10 R: TGATGGCCTTCGATTCTGGATT (SEQ ID NO:77); DUX4 F: CGGAGAACTGCCATTCTTTC (SEQ ID NO:78) (Shadle et al., 2017); DUX4 R: CAGCCAGAATTTCACGGAAG (SEQ ID NO:79); HLA-A F: CGACGCCGCGAGCCAGA (SEQ ID NO:80) (Kruse et al., 2015); HLA-A R: GCGATGTAATCCTTGCCGTCGTAG (SEQ ID NO:81); HLA-B F: CTACCCTGCGGAGATCA (SEQ ID NO:82) (Meissner et al., 2010); HLA-B R: ACAGCCAGGCCAGCAACA (SEQ ID NO:83); HLA-C F: GGAGACACAGAAGTACAAGCG (SEQ ID NO:84) (Kruse et al., 2015); HLA-C R: CGTCGTAGGCGTACTGGTCATA (SEQ ID NO:85); PRPF8 F: ACCCAATCTCCCATAGGCAC (SEQ ID NO:86) (Zeisel et al., 2013); PRPF8 R: AGGAAGGGCTCCACAAACTC (SEQ ID NO:87); RPL13A F: AACCTCCTCCTTTTCCAAGC (SEQ ID NO:88) (Geng et al., 2012); RPL13A R: GCAGTACCTGTTTAGCCACGA (SEQ ID NO:89); RPL27 F: GCAAGAAGAAGATCGCCAAG (SEQ ID NO:90) (Shadle et al., 2017)/RPL27 R: TCCAAGGGGATATCCACAGA (SEQ ID NO:91); TRIM43 F: ACCCATCACTGGACTGGTGT (SEQ ID NO:92) (Geng et al., 2012); TRIM43 R: CACATCCTCAAAGAGCCTGA (SEQ ID NO:93); ZSCAN4 F: TGGAAATCAAGTGGCAAAAA (SEQ ID NO:94) (Geng et al., 2012); ZSCAN4 R: CTGCATGTGGACGTGGAC (SEQ ID NO:95).

20. SiRNAs

The following siRNAs were used: siCTRL: AATTCTCCGAACGTGTCACGT (SEQ ID NO:96); siPRPF8-1: ACGGGCATGTATCGATACAAA (SEQ ID NO:97); siPRPF8-2: ATGGCTTGTCATCCTGAATAA (SEQ ID NO:98); siPRPF8-3: CAACGTCGTCATCAACTATAA (SEQ ID NO:99); siPRPF8-4: CTCATCGTGGACCACAACATA (SEQ ID NO:100)

21. Survival Analyses.

Survival analyses and corresponding statistical tests were performed with the Kaplan-Meier estimator and log-rank test as implemented in the R package survival (Therneau and Grambsch, 2000).

F. Quantification and Statistical Analysis

1. Data Analysis and Visualization

Data analysis was performed in the R programming environment and relied on Bioconductor (Huber et al., 2015), dplyr (Wickham and Francois), and ggplot2 (Wickham, 2009). The RT-qPCR panels were generated using GraphPad Prism Software (version 7.0, www.graphpad.com). Flow cytometry data was analyzed with FloJo (version 10.5.3, www.flowjo.com/solutions/flowjo).

G. Tables

TABLE 1
Key Resources

| REAGENT or RESOURCE | SOURCE | IDENTIFIER |
|---|---|---|
| Antibodies | | |
| GAPDH (6C5) | GeneTex, Inc. | GTX28245 |
| | | RRID: AB_370675 |
| MHC Class I (F-3) | Santa Cruz | sc-55582 |
| | Biotechnology | RRID: AB_831547 |
| FITC anti-human HLA-A,B,C (W6/32) | BioLegend | 311404 |
| | | RRID: AB_314873 |
| ZSCAN4 | Invitrogen | PA5-32106 |
| | | RRID: AB_2549579 |
| DUX4 (14-3) | Geng et al., 2011 | N/A |
| Peroxidase AffiniPure Goat | Jackson | 115-035-146 |
| Anti-Mouse IgG (H + L) | ImmunoResearch | RRID: AB_2307392 |
| Peroxidase AffiniPure Goat | Jackson | 111-035-144 |
| Anti-Rabbit (H + L) | ImmunoResearch | RRID: AB_2307391 |
| Chemicals, Peptides, and Recombinant Proteins | | |
| Insulin | Sigma-Aldrich | I1882 |
| Recombinant human basic fibroblast growth factor | Promega | G5071 |
| Dexamethasone | Sigma-Aldrich | D4902 |
| Puromycin dihydrochloride | Sigma-Aldrich | P8833 |
| Doxycycline hyclate | Sigma-Aldrich | D9891 |
| Recombinant human IFN-gamma | R&D Systems | 2851F100 |
| Polybrene | Sigma-Aldrich | 107689 |
| Penicillin/streptomycin | Gibco/Thermo Fisher | 15140122 |
| Sodium Pyruvate | Gibco/Thermo Fisher | 11360070 |
| DNase 1 | Gibco/Thermo Fisher | 18068015 |
| MEM Non-Essential Amino Acids Solution | Gibco/Thermo Fisher | 11140050 |
| Critical Commercial Assays | | |
| Lipofectamine RNAiMAX | Life Technologies | 13778150 |
| SuperScriptIII First-Strand System | Life Technologies | 18080051 |
| iTaq SYBR Green Supermix | Bio-Rad | 1725124 |
| TruSeq RNA Library Preparation Kit | Illumine | RS-122-2001 |
| NucleoSpin RNA kit | Machery-Nagel | 740955 |
| Key Instruments | | |
| QuantStudio 7 Flex | Applied Biosystems | N/A |
| 4200 TapeStation System | Agilent Technologies | N/A |
| Trinean DropSense 96 UV-Vis spectrophotometer | Caliper Life Sciences | N/A |
| Qubit 2.0 Fluorometer | Life Technologies | N/A |
| HiSeq 2500 | Illumine | N/A |
| LSRFortessa X-50 | BD Biosciences | N/A |
| Source Data | | |
| The Cancer Genome Atlas RNA-seq data | CGHub | N/A |
| | RRID: SCR_002657 | |
| RNA-seq data for Eidahl et al., 2016; | NCBI Gene Expression | GSE85632, |
| Hendrickson et al., 2017; | Omnibus | GSE85935, |
| Hugo et al., 2016 | RRID: SCR_005012 | GSE78220 |
| RNA-seq data for Rickard et al., 2015 | NCBI Sequence Read | SRP058319 |
| | Archive | |
| | RRID: SCR_004891 | |
| RNA-seq data for Van Allen et al., 2015 | dbGaP | ph5000452.v2.p1 |
| | RRID: SCR_002709 | |
| RNA-seq data for Yasuda et al., 2016 | Japanese Genotype- | JGAS00000000047 |
| | Phenotype Archive | |
| | RRID: SCR_003118 | |
| RNA-seq data for Lilljebjörn et al., 2016 | European Genome- | EGAD00001002112 |
| | phenome Archive | |
| | RRID: SCR_004944 | |
| GTEx Sample Annotations and Gene Expression | GTEx portal | www.gtexportal.org |
| | RRID: SCR_013042 | |
| ChIP-seq data for Geng et al., 2012 | NCBI Gene Expression | GSE33838 |
| | Omnibus | |
| | RRID: SCR_005012 | |
| Deposited Data | | |
| RNA-seq data for this study | NCBI Gene Expression | GSE128917 |
| | Omnibus | |
| | RRID: SCR_005012 | |
| Experimental Models: Cell Lines | | |
| MB135 cells | Snider et al., 2010 | N/A |
| MB2401 cells | Campbell et al., 2018 | N/A |
| MB073 cells | Campbell et al., 2018 | N/A |
| A204 cells | ATCC | HTB-82 |
| | | RRID: CVCL_1058 |
| HeLa cells | ATCC | CCL-2 |
| | | RRID: CVCL_0030 |
| MCF-7 cells | ATCC | HTB-22 |
| | | RRID: CVCL_0031 |
| Mel375 and Mel526 cells | Dr. Seth Pollack | N/A |
| | (Pollack et al., 2012) | |
| SuSa cells | DSMZ | ACC 747 |
| | | RRID: CVCL_L280 |
| MB135iDUX4 cells | Jagannathan et al., 2016 | N/A |
| MB135iDUXB cells | This study | N/A |
| A204iDUX4 cells | This study | N/A |
| HeLaiDUX4 cells | This study | N/A |
| MCF-7iDUX4 cells | This study | N/A |
| Mel375iDUX4 cells | This study | N/A |
| Mel526iDUX4 cells | This study | N/A |
| SuSaiDUX4 cells | This study | N/A |
| Oligonucleotides | | |
| Primers for qPCR and cloning | This study | N/A |
| See STAR★Methods for sequences | | |
| FlexiTube GeneSolution siRNAs targeting PRPF8 | Qiagen | GS10594 |
| See STAR★Methods for sequences | | |
| Non-targeting Negative Control siRNA | Qiagen | 1022076 |
| See STAR★Methods for sequence | | |
| Recombinant DNA | | |
| pCW57.1 | Addgene | 41397 |
| | | RRID: Addgene_41397 |
| pCW57.1-DUX4-CA | Addgene | 99281 |
| | | RRID: Addgene_99281 |
| pCW57.1-DUXB-CA | This study, available | 125027 |
| | on Addgene | RRID: Addgene_125027 |
| Software and Algorithms | | |
| Bowtie v1.0.0 | Langmead et al, 2009 | github.com/BenLangmead/ |
| | RRID: SCR_005476 | bowtie/ |
| RSEM v.1.2.4 | Li and Dewey, 2011 | deweylab.github.io/RSEM/ |
| | RRID: SCR_013027 | |
| TopHat v2.0.8b | Trapnell et al, 2009 | ccb.jhu.edu/software/tophat/ |
| | RRID: SCR_013035 | index.shtml |
| MISO v2.0 | Katz et al., 2010 | genes.mit.edu/burgelab/ |
| | RRID: SCR_003124 | miso/ |
| Samtools v1.3.1 | Li et al., 2009 | www.htslib.org |
| | RRID: SCR_002105 | |
| Kallisto v0.43.0 | Bray et al., 2016 | pachterlab.github.io/ |
| | RRID: SCR_016582 | kallisto/ |
| MACS v2.1.1.20160309 | Zhang et al., 2008 | github.com/taoliu/MACS/ |
| | RRID: SCR_013291 | |
| IGV v2.3.90 | Thorvaldsdottir et al., 2013 | software.broadinstitute.org/ |
| | RRID: SCR_011793 | software/igv/ |
| Bioconductor | Huber et al., 2015 | www.bioconductor.org |
| | RRID: SCR_006442 | |
| GOseq | Young et al., 2010 | bioconductor.org/packages/ |
| | RRID: SCR_017052 | release/bioc/html/goseq.html |
| dplyr | Wickham and Francois | cran.r-project.org/ |
| | RRID: SCR_016708 | web/packages/dplyr/ |
| | | index.html |
| ggplot2 | Wickham et al., 2009 | ggplot2.org |
| | RRID: SCR 014601 | |
| GraphPad Prism v7.0 | RRID: SCR_002798 | www.graphpad.com |
| FlowJo v10.5.3 | RRID: SCR_008520 | www.flowjo.com/solutions/ |
| | | flowjo |
| BD FACSDiva | RRID: SCR_001456 | www.bdbiosciences.com/ |
| | | instruments/software/ |
| | | facsdiva/index.jsp |

TABLE S3
Table of high-confidence DUX4 targets (DUX4-induced
genes and repetitive elements illustrated in FIG. 3B).

| Type | Gene Name | Gene ID |
|---|---|---|
| gene | CCNA1 | ENSG00000133101 |
| gene | CTD-2611012.2 | ENSG00000229292 |
| gene | DUXA | ENSG00000258873 |
| gene | DUXB | DUXB |
| gene | HNRNPCL1 | ENSG00000179172 |
| gene | KDM4E | ENSG00000235268 |
| gene | KHDC1L | ENSG00000256980 |
| gene | LEUTX | ENSG00000213921 |
| gene | MBD3L2 | ENSG00000230522 |
| gene | MBD3L3 | ENSG00000182315 |
| gene | MBD3L5 | ENSG00000237247 |
| gene | PRAMEF1 | ENSG00000116721 |
| gene | PRAMEF11 | ENSG00000204513 |
| gene | PRAMEF12 | ENSG00000116726 |
| gene | PRAMEF13 | ENSG00000204495 |
| gene | PRAMEF14 | ENSG00000204481 |
| gene | PRAMEF15 | ENSG00000157358 |
| gene | PRAMEF2 | ENSG00000120952 |
| gene | PRAMEF20 | ENSG00000204478 |
| gene | PRAMEF4 | ENSG00000243073 |
| gene | PRAMEF6 | ENSG00000232423 |
| gene | PRAMEF7 | ENSG00000204510 |
| gene | PRAMEF9 | ENSG00000204501 |
| gene | RFPL1 | ENSG00000128250 |
| gene | RFPL2 | ENSG00000128253 |
| gene | RFPL4A | ENSG00000223638 |
| gene | RFPL4B | ENSG00000251258 |
| gene | SLC34A2 | ENSG00000157765 |
| gene | TRIM43 | ENSG00000144015 |
| gene | TRIM48 | ENSG00000150244 |
| gene | TRIM49 | ENSG00000168930 |
| gene | TRIM49B | ENSG00000182053 |
| gene | TRIM49C | ENSG00000204449 |
| gene | TRIM51BP | ENSG00000204455 |
| gene | TRIM53AP | ENSG00000225581 |
| gene | TRIM53BP | ENSG00000166013 |
| gene | W12-2994D6.2 | ENSG00000229571 |
| gene | W12-3308P17.2 | ENSG00000239810 |
| gene | ZSCAN4 | ENSG00000180532 |
| gene | ZSCAN5B | ENSG00000197213 |
| gene | ZSCAN5C | ENSG00000204532 |
| gene | ZSCAN5D | ENSG00000267908 |
| repetitive element | HERVL | HERVL |
| repetitive element | LSAU | LSAU |
| repetitive element | MLT2A1 | MLT2A1 |
| repetitive element | SVA_D | SVA_D |
| repetitive element | SVA_E | SVA_E |

TABLE S4
Table of high-confidence DUXB targets
(DUXB-induced genes illustrated in FIG. 9C).

| Gene | |
|---|---|
| Name | Gene ID |
| ACTA2 | ENSG00000107796 |
| ADAP1 | ENSG00000105963 |
| AKAP6 | ENSG00000151320 |
| C21orf91 | ENSG00000154642 |
| CCDC39 | ENSG00000145075 |
| CENPT | ENSG00000102901 |
| CTGF | ENSG00000118523 |
| CXCL12 | ENSG00000107562 |
| DKK2 | ENSG00000155011 |
| FAM20C | ENSG00000177706 |
| IER5L | ENSG00000188483 |
| IGF2-AS | ENSG00000099869 |
| IGFBP2 | ENSG00000115457 |
| JAG1 | ENSG00000101384 |
| LOXL4 | ENSG00000138131 |
| LURAP1L | ENSG00000153714 |
| MAOA | ENSG00000189221 |
| NID2 | ENSG00000087303 |
| NR2F1 | ENSG00000175745 |
| NUDT10 | ENSG00000122824 |
| PADI2 | ENSG00000117115 |
| PDLIM3 | ENSG00000154553 |
| PTGER2 | ENSG00000125384 |
| PTGES | ENSG00000148344 |
| PYGL | ENSG00000100504 |
| TNC | ENSG00000041982 |
| TRIM55 | ENSG00000147573 |
| TRIM9 | ENSG00000100505 |
| TUBB3 | ENSG00000258947 |

### Example 2: Treatment of Cells with DUX4 Inhibitory Agents

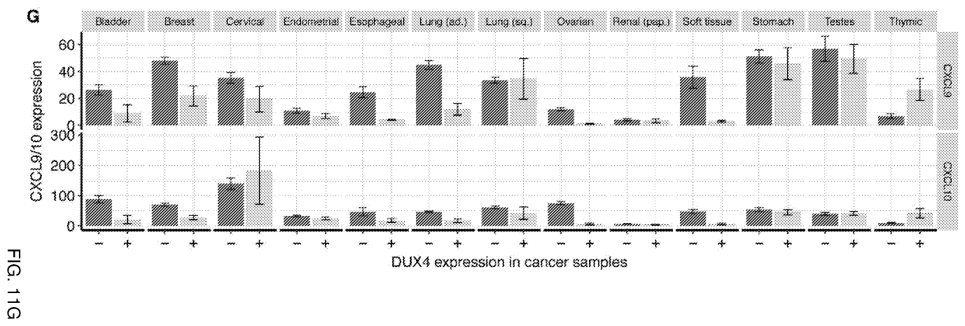

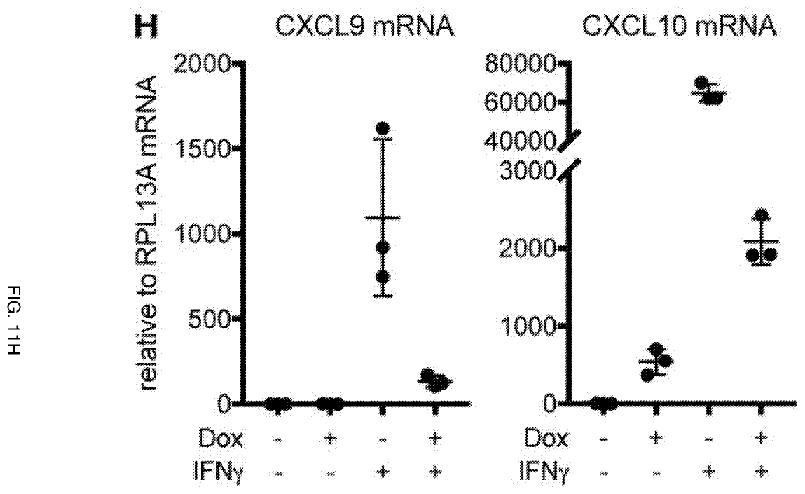

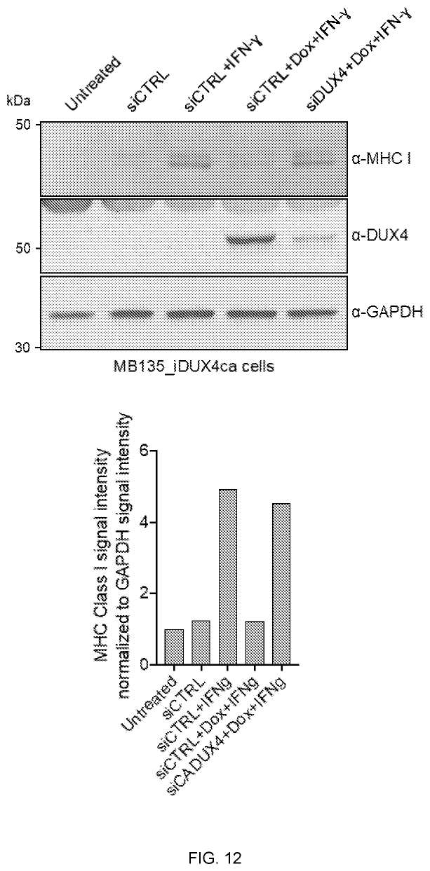

The demonstration that DUX4 expression suppresses MHC Class I gene expression in response to INF-γ leads to the prediction that agents or drugs that suppress DUX4 expression will result in MHC Class I expression at higher levels. To test this, DUX4 expression was induced in MB135 myoblasts, and the cells were then exposed to IFN-γ either with or without the suppression of DUX4 using siRNA against the DUX4 mRNA. As shown in FIG. 12, cells treated with the siRNA targeting DUX4 showed significantly higher expression of the MHC Class I genes. Showing that the siRNA targeted to DUX4 results in higher MHC Class I expression provides proof-of-principle that any drug or agent that suppresses DUX4 will result in the higher expression of MHC Class I genes in DUX4 expressing cancers or tissues.

### Example 3: Treatment of Cancer Cells with a CDK7 Inhibitor Inhibits DUX4 Expression

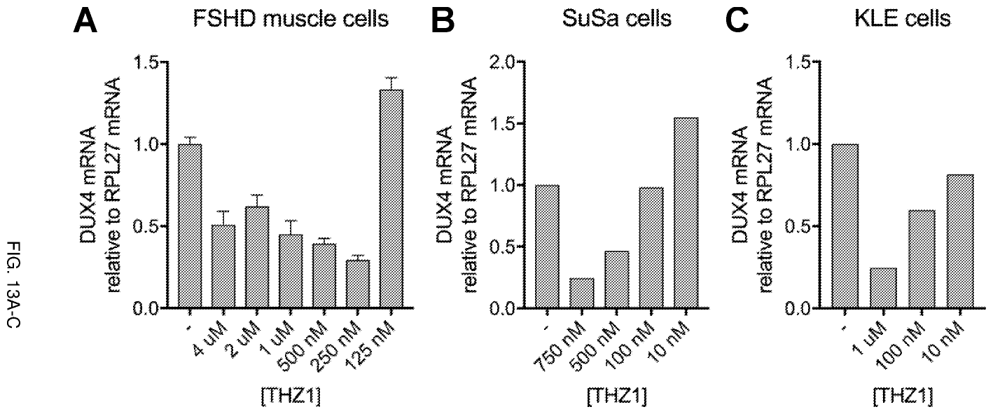

Differentiating MB200 FSHD2 patient-derived muscle cells, SuSa testicular teratocarcinoma cells, or KLE endometrial adenocarcinoma cells were treated with vehicle (−, DMSO) or varying concentrations of THZ1, as indicated in FIG. 13, for 24 hours (FIG. 13A) or 20 hours (FIG. 13B-C). Next, the cells were analyzed for DUX4 mRNA levels by RT-qPCR. SuSa and KLE are cancer cell lines expressing DUX4 and THZ1 inhibits DUX4 expression in these cancer cell lines at similar concentrations as the inhibition of DUX4 in FSHD muscle cells, indicating that some drugs developed for FSHD therapies might also be effective at inhibiting DUX4 in different cancers.

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Almeida L G, Sakabe N J, de Oliveira A R, Silva M C C, Mundstein A S, Cohen T, Chen Y T, Chua R, Gurung S, Gnjatic S, et al. 2009. CTdatabase: A knowledge-base of high-throughput and curated data on cancer-testis antigens. Nucleic Acids Res 37: D816-9.
Arahata K, Ishihara T, Fukunaga H, Orimo S, Lee R I, Goto K, Nonaka I. 1995. Inflammatory response in facioscapulohumeral muscular dystrophy (FSHD): Immunocytochemical and genetic analyses. Muscle Nerve 18: S56-S66.
Ben-Porath I, Thomson M W, Carey V J, Ge R, Bell G W, Regev A, The inventorsinberg R A. 2008. An embryonic stem cell-like gene expression signature in poorly differentiated aggressive human tumors. Nat Genet 40: 499-507.
Blewitt M E, Vickaryous N K, Hemley S J, Ashe A, Bruxner T J, Preis J I, Arkell R, Whitelaw E. 2005. An N-ethyl-N-nitrosourea screen for genes involved in variegation in the mouse. Proc Natl Acad Sci 102: 7629-7634.
Bray N L, Pimentel H, Melsted P, Pachter L. 2016. Near-optimal probabilistic RNA-seq quantification. Nat Biotechnol 34: 525-527.
Bruggeman J W, Koster J J B, Repping S, Hamer G. 2017. Massive expression of germ cell specific genes is a hallmark of cancer and a potential target for novel treatment development. bioRxiv. https://doi. org/10.1101/195305.
Caballero O L, Chen Y T. 2009. Cancer/testis (C T) antigens: Potential targets for immunotherapy. Cancer Sci 100: 2014-2021.
Chang K, Creighton C J, Davis C, Donehower L, Drummond J, Wheeler D, Ally A, Balasundaram M, Birol I, Butterfield Y S N, et al. 2013. The Cancer Genome Atlas Pan-Cancer analysis project. Nat Genet 45: 1113-1120.
Chang T C, Yang Y, Yasue H, Bharti A K, Retzel E F, Liu W S. 2011. The expansion of the PRAME gene family in Eutheria. PLoS One 6: e16867.
Choi S H, Gearhart M D, Cui Z, Bosnakovski D, Kim M, Schennum N, Kyba M. 2016. DUX4 recruits p300/CBP through its C-terminus and induces global H3K27 acetylation changes ed. R. Green. Nucleic Acids Res 44: 5161-5173.
Daxinger L, Harten S K, Oey H, Epp T, Isbel L, Huang E, Whitelaw N, Apedaile A, Sorolla A, Yong J, et al. 2013. An ENU mutagenesis screen identifies novel and known genes involved in epigenetic processes in the mouse. Genome Biol 14: R96.
de Greef J C, Lemmers R J L F, van Engelen B G M, Sacconi S, Venance S L, Frants R R, Tawil R, van der Maarel S M. 2009. Common epigenetic changes of D4Z4 in contraction-dependent and contraction-independent FSHD. Hum Mutat 30: 1449-1459.
De Iaco A, Planet E, Coluccio A, Verp S, Duc J, Trono D. 2017. DUX-family transcription factors regulate zygotic genome activation in placental mammals. Nat Genet 49: 941-945.
Dvinge H, Bradley R K. 2015. Widespread intron retention diversifies most cancer transcriptomes. Genome Med 7: 45.
Dvinge H, Ries R E, Ilagan J O, Stirewalt D L, Meshinchi S, Bradley R K. 2014. Sample processing obscures cancer-specific alterations in leukemic transcriptomes. Proc Natl Acad Sci 111: 16802-16807.
Eidahl J O, Giesige C R, Domire J S, Wallace L M, Fowler A M, Guckes S M, Garwick-Coppens S E, Labhart P, Harper S Q. 2016. Mouse Dux is myotoxic and shares partial functional homology with its human paralog DUX4. Hum Mol Genet ddw287.
Feng Q, Snider L, Jagannathan S, Tawil R, van der Maarel S M, Tapscott S J, Bradley R K. 2015. A feedback loop between nonsense-mediated decay and the retrogene DUX4 in facioscapulohumeral muscular dystrophy ed. R. Green. Elife 2015: e04996.
Flicek P, Ahmed I, Amode M R, Barrell D, Beal K, Brent S, Carvalho-Silva D, Clapham P, Coates G, Fairley S, et al. 2013. Ensembl 2013. Nucleic Acids Res 41: D48-55.
Frisullo G, Frusciante R, Nociti V, Tasca G, Renna R, Iorio R, Patanella A K, Iannaccone E, Marti A, Rossi M, et al. 2011. CD8+ T cells in facioscapulohumeral muscular dystrophy patients with inflammatory features at muscle MRI. J Clin Immunol 31: 155-166.
Gabriels J, Beckers M C, Ding H, De Vriese A, Plaisance S, van der Maarel S M, Padberg G W, Frants R R, Hewitt J E, Collen D, et al. 1999. Nucleotide sequence of the partially deleted D4Z4 locus in a patient with FSHD identifies a putative gene within each 3.3 kb element. Gene 236: 25-32.
Geng L N, Yao Z, Snider L, Fong A P, Cech J N, Young J M, vanderMaarel S M, Ruzzo W L, Gentleman R C, Tawil R, et al. 2012. DUX4 Activates Germline Genes, Retroelements, and Immune Mediators: Implications for Facioscapulohumeral Dystrophy. Dev Cell 22: 38-51.
GTEx Consortium. 2015. Human genomics. The Genotype-Tissue Expression (GTEx) pilot analysis: multitissue gene regulation in humans. Science 348: 648-660.
Haider S, McIntyre A, van Stiphout RGPM, Winchester L M, Wigfield S, Harris A L, Buffa F M. 2016. Genomic alterations underlie a pan-cancer metabolic shift associated with tumour hypoxia. Genome Biol 17: 140.
Hanahan D, The inventorsinberg R A. 2000. The hallmarks of cancer. Cell 100: 57-70.
Hayashi H, Arao T, Togashi Y, Kato H, Fujita Y, De Velasco M A, Kimura H, Matsumoto K, Tanaka K, Okamoto I, et al. 2015. The OCT4 pseudogene POU5F1B is amplified and promotes an aggressive phenotype in gastric cancer. Oncogene 34: 199-208.
Hendrickson P G, Doráis J A, Grow E J, Whiddon J L, Lim J W, Wike C L, The inventorsaver B D, Pflueger C, Emery B R, Wilcox A L, et al. 2017. Conserved roles of mouse DUX and human DUX4 in activating cleavage-stage genes and MERVL/HERVL retrotransposons. Nat Genet 49: 925-934.
Hill V K, Kim J S, Waldman T. 2016. Cohesin mutations in human cancer. Biochim Biophys Acta-Rev Cancer 1866: 1-11.
Hoadley K A, Yau C, Wolf D M, Cherniack A D, Tamborero D, Ng S, Leiserson M D M, Niu B, McLellan M D, Uzunangelov V, et al. 2014. Multiplatform analysis of 12 cancer types reveals molecular classification within and across tissues of origin. Cell 158: 929-944.
Huber W, Carey V J, Gentleman R, Anders S, Carlson M, Carvalho B S, Bravo H C, Davis S, Gatto L, Girke T, et al. 2015. Orchestrating high-throughput genomic analysis with Bioconductor. Nat Methods 12: 115-121.
Ikeda H, Lethe B, Lehmann F, Van Baren N, Baurain J F, De Smet C, Chambost H, Vitale M, Moretta A, Boon T, et al. 1997. Characterization of an antigen that is recognized on a melanoma showing partial HLA loss by CTL expressing an N K inhibitory receptor. Immunity 6: 199-208.
Kaczkowski B, Tanaka Y, Kawaji H, Sandelin A, Andersson R, Itoh M, Lassmann T, Hayashizaki Y, Carninci P, Forrest A R R. 2016. Transcriptome analysis of recurrently deregulated genes across multiple cancers identifies new pan-cancer biomarkers. Cancer Res 76: 216-226.
Katz Y, Wang E T, Airoldi E M, Burge C B. 2010. Analysis and design of RNA sequencing experiments for identifying isoform regulation. Nat Methods 7: 1009-1015.
Kawamura-Saito M, Yamazaki Y, Kaneko K, Kawaguchi N, Kanda H, Mukai H, Gotoh T, Motoi T, Fukayama M, Aburatani H, et al. 2006. Fusion between CIC and DUX4 up-regulates PEA3 family genes in Ewing-like sarcomas with t(4;19)(q35;q13) translocation. Hum Mol Genet 15: 2125-2137.
Kim M-S, Pinto S M, Getnet D, Nirujogi R S, Manda S S, Chaerkady R, Madugundu A K, Kelkar D S, Isserlin R, Jain S, et al. 2014. A draft map of the human proteome. Nature 509: 575-581.
Kosti I, Jain N, Aran D, Butte A J, Sirota M. 2016. Cross-tissue Analysis of Gene and Protein Expression in Normal and Cancer Tissues. Sci Rep 6: 24799.
Langmead B, Trapnell C, Pop M, Salzberg S L. 2009. Ultrafast and memory-efficient alignment of short DNA sequences to the human genome. Genome Biol 10: R25.
Lee J H, Goto K, Matsuda C, Arahata K. 1995. Characterization of a tandemly repeated 3.3-kb Kpnl unit in the facioscapulohumeral muscular dystrophy (FSHD) gene region on chromosome 4g35. Muscle Nerve 18: S6-S13.
Leiserson M D M, Vandin F, Wu H T, Dobson J R, Eldridge J V., Thomas J L, Papoutsaki A, Kim Y, Niu B, McLellan M, et al. 2015. Pan-cancer network analysis identifies combinations of rare somatic mutations across pathways and protein complexes. Nat Genet 47: 106-114.
Lemmers R J L F, Tawil R, Petek L M, Balog J, Block G J, Santen G W E, Amell A M, Van Der Vliet P J, Almomani R, Straasheijm K R, et al. 2012. Digenic inheritance of an SMCHD1 mutation and an FSHD-permissive D4Z4 allele causes facioscapulohumeral muscular dystrophy type 2. Nat Genet 44: 1370-1374.
Lemmers R J L F, van der Vliet P J, Klooster R, Sacconi S, Camano P, Dauwerse J G, Snider L, Straasheijm K R, Jan van Ommen G, Padberg G W, et al. 2010. A Unifying Genetic Model for Facioscapulohumeral Muscular Dystrophy. Science (80-) 329: 1650-1653.
Li B, Dewey C N. 2011. RSEM: Accurate transcript quantification from RNA-Seq data with or without a reference genome. BMC Bioinformatics 12.
Li T, Fan J, Wang B, Traugh N, Chen Q, Liu J S, Li B, Liu X S. 2017. TIMER: A web server for comprehensive analysis of tumor-infiltrating immune cells. Cancer Res 77: e108-e110.
Lilljebjorn H, Henningsson R, Hyrenius-Wittsten A, Olsson L, Orsmark-Pietras C, von Palffy S, Askmyr M, Rissler M, Schrappe M, Cario G, et al. 2016. Identification of ETV6-RUNX1-like and DUX4-rearranged subtypes in paediatric B-cell precursor acute lymphoblastic leukaemia. Nat Commun 7: 11790.
Liu Y F, Wang B Y, Zhang W N, Huang J Y, Li B S, Zhang M, Jiang L, Li J F, Wang M J, Dai Y J, et al. 2016. Genomic Profiling of Adult and Pediatric B-cell Acute Lymphoblastic Leukemia. EBioMedicine 8: 173-183.
Mak M P, Tong P, Diao L, Cardnell R J, Gibbons D L, William W N, Skoulidis F, Parra E R, Rodriguez-Canales J, Wistuba I I, et al. 2016. A Patient-Derived, Pan-Cancer EMT Signature Identifies Global Molecular Alterations and Immune Target Enrichment Following Epithelial-to-Mesenchymal Transition. Clin Cancer Res 22: 609-620.
Meyer L R, Zweig A S, Hinrichs A S, Karolchik D, Kuhn R M, Wong M, Sloan C A, Rosenbloom K R, Roe G, Rhead B, et al. 2013. The UCSC Genome Browser database: Extensions and updates 2013. Nucleic Acids Res 41.
Monk M, Holding C. 2001. Human embryonic genes re-expressed in cancer cells. Oncogene 20: 8085-8091.
Pierce G B. 1983. The cancer cell and its control by the embryo. Rous-Whipple Award lecture. Am J Pathol 113: 117-24.
Qian M, Zhang H, Kham S K Y, Liu S, Jiang C, Zhao X, Lu Y, Goodings C, Lin T N, Zhang R, et al. 2017. Whole-transcriptome sequencing identifies a distinct subtype of acute lymphoblastic leukemia with predominant genomic abnormalities of EP300 and CREBBP. Genome Res 27: 185-195.
Rhodes D R, Yu J, Shanker K, Deshpande N, Varambally R, Ghosh D, Barrette T, Pandey A, Chinnaiyan A M. 2004. Large-scale meta-analysis of cancer microarray data identifies common transcriptional profiles of neoplastic transformation and progression. Proc Natl Acad Sci 101: 9309-9314.
Rickard A M, Petek L M, Miller D G. 2015. Endogenous DUX4 expression in FSHD myotubes is sufficient to cause cell death and disrupts RNA splicing and cell migration pathways. Hum Mol Genet 24: 5901-5914.
Robinson M D, Oshlack A. 2010. A scaling normalization method for differential expression analysis of RNA-seq data. Genome Biol 11: R25.
Rooney M S, Shukla S A, Wu C J, Getz G, Hacohen N. 2015. Molecular and genetic properties of tumors associated with local immune cytolytic activity. Cell 160: 48-61.
Sacconi S, Lemmers R J L F, Balog J, van der Vliet P J, Lahaut P, van Nieuwenhuizen M P, Straasheijm K R, Debipersad R D, Vos-Versteeg M, Salviati L, et al. 2013. The FSHD2 Gene SMCHD1 Is a Modifier of Disease Severity in Families Affected by FSHD1. Am J Hum Genet 93: 744-751.
Shukla S A, Rooney M S, Rajasagi M, Tiao G, Dixon P M, Lawrence M S, Stevens J, Lane W J, Dellagatta J L, Steelman S, et al. 2015. Comprehensive analysis of cancer-associated somatic mutations in class i HLA genes. Nat Biotechnol 33: 1152-1158.
Smith H A, McNeel D G. 2010. The SSX family of cancer-testis antigens as target proteins for tumor therapy. Clin Dev Immunol 2010: 150591.
Snider L, Geng L N, Lemmers R J L F, Kyba M, Ware C B, Nelson A M, Tawil R, Filippova G N, van der Maarel S M, Tapscott S J, et al. 2010. Facioscapulohumeral dystrophy: Incomplete suppression of a retrotransposed gene. PLoS Genet 6: 1-14.
Tam R, Smith K P, Lawrence J B. 2004. The 4q subtelomere harboring the FSHD locus is specifically anchored with peripheral heterochromatin unlike most human telomeres. J Cell Biol 167: 269-279.
Thorvaldsdottir H, Robinson J T, Mesirov J P. 2013. Integrative Genomics Viewer (IGV): high-performance genomics data visualization and exploration. Brief Bioinform 14: 178-192.
Trapnell C, Pachter L, Salzberg S L. 2009. TopHat: Discovering splice junctions with RNA-Seq. Bioinformatics 25: 1105-1111.
Uhlen M, Zhang C, Lee S, Sjostedt E, Fagerberg L, Bidkhori G, Benfeitas R, Arif M, Liu Z, Edfors F, et al. 2017. A pathology atlas of the human cancer transcriptome. Science (80-) 357.
Van Allen E M, Miao D, Schilling B, Shukla S A, Blank C, Zimmer L, Sucker A, Hillen U, Foppen M H G, Goldinger S M, et al. 2015. Genomic correlates of response to CTLA-4 blockade in metastatic melanoma. Science (80-) 350: 207-211.
Van Den Boogaard M L, Lemmers R J L F, Balog J, Wohlgemuth M, Auranen M, Mitsuhashi S, Van Der Vliet P J, Straasheijm K R, Van Den Akker R F P, Kriek M, et al. 2016. Mutations in DNMT3B Modify Epigenetic Repression of the D4Z4 Repeat and the Penetrance of Facioscapulohumeral Dystrophy. Am J Hum Genet 98: 1020-1029.
Van Overveld P G M, Lemmers R J F L, Sandkuijl L A, Enthoven L, Winokur S T, Bakels F, Padberg G W, Van Ommen G J B, Frants R R, Van Der Maarel S M. 2003. Hypomethylation of D4Z4 in 4q-linked and non-4q-linked facioscapulohumeral muscular dystrophy. Nat Genet 35: 315-317.
Whiddon J L, Langford A T, Wong C-J, Zhong J W, Tapscott S J. 2017. Conservation and innovation in the DUX4-family gene network. Nat Genet 49: 935-940.
Wickham H. 2009. ggplot2. 1st ed. Springer, New York.
Wickham H, Francois R. 2015. dplyr: A Grammar of Data Manipulation. R Packag version 042 3.
Wijmenga C, Frants R R, Hewitt J E, van Deutekom J C T, van Geel M, Wright T J, Padberg G W, Hofker M H, van Ommen G-J B. 1993. Molecular genetics of facioscapulohumeral muscular dystrophy. Neuromuscul Disord 3: 487-491.
Yasuda T, Tsuzuki S, Kawazu M, Hayakawa F, Kojima S, Ueno T, Imoto N, Kohsaka S, Kunita A, Doi K, et al. 2016. Recurrent DUX4 fusions in B cell acute lymphoblastic leukemia of adolescents and young adults. Nat Genet 48: 569-574.
Young J M, Whiddon J L, Yao Z, Kasinathan B, Snider L, Geng L N, Balog J, Tawil R, van der Maarel S M, Tapscott S J. 2013. DUX4 Binding to Retroelements Creates Promoters That Are Active in FSHD Muscle and Testis. PLoS Genet 9.
Young M D, Wakefield M J, Smyth G K, Oshlack A. 2010. Gene ontology analysis for RNA-seq: accounting for selection bias. Genome Biol 11: R14.
Yu X, Lian B, Wang L, Zhang Y, Dai E, Meng F, Liu D, Wang S, Liu X, Wang J, et al. 2014. The pan-cancer analysis of gene expression patterns in the context of inflammation. Mol Biosyst 10: 2270-6.
Zalzman M, Falco G, Sharova L V., Nishiyama A, Thomas M, Lee S L, Stagg C A, Hoang H G, Yang H Te, Indig F E, et al. 2010. Zscan4 regulates telomere elongation and genomic stability in E S cells. Nature 464: 858-863.
Zeng W, De Greef J C, Chen Y Y, Chien R, Kong X, Gregson H C, Winokur S T, Pyle A, Robertson K D, Schmiesing J A, et al. 2009. Specific loss of histone H3 lysine 9 trimethylation and HP1γ/cohesin binding at D4Z4 repeats is associated with facioscapulohumeral dystrophy (FSHD). PLoS Genet 5: e1000559.

### EXAMPLE 1 REFERENCES

Adzhubei, I. A., Schmidt, S., Peshkin, L., Ramensky, V. E., Gerasimova, A., Bork, P., Kondrashov, A. S., and Sunyaev, S. R. (2010). A method and server for predicting damaging missense mutations. Nat Methods 7, 248-249.
Arahata, K., Ishihara, T., Fukunaga, H., Orimo, S., Lee, J. H., Goto, K., and Nonaka, I. (1995). Inflammatory response in facioscapulohumeral muscular dystrophy (FSHD): immunocytochemical and genetic analyses. Muscle Nerve Suppl 2, S56-S66.
Ben-Porath, I., Thomson, M. W., Carey, V. J., Ge, R., Bell, G. W., Regev, A., and Weinberg, R. A. (2008). An embryonic stem cell-like gene expression signature in poorly differentiated aggressive human tumors. Nat Genet 40, 499-507.
Bray, N. L., Pimentel, H., Melsted, P., and Pachter, L. (2016). Near-optimal probabilistic RNA-seq quantification. Nat Biotechnol 34, 525-527.
Caballero, O. L., and Chen, Y.-T. (2009). Cancer/testis (CT) antigens: potential targets for immunotherapy. Cancer Sci. 100, 2014-2021.
Campbell, A. E., Shadle, S. C., Jagannathan, S., Lim, J.-W., Resnick, R., Tawil, R., van der Maarel, S. M., and Tapscott, S. J. (2018). NuRD and CAF-1-mediated silencing of the D4Z4 array is modulated by DUX4-induced MBD3L proteins. Elife 7, e31023.
Chang, T.-C., Yang, Y., Yasue, H., Bharti, A. K., Retzel, E. F., and Liu, W.-S. (2011). The expansion of the PRAME gene family in Eutheria. PLoS ONE 6, e16867.
Choi, S.-H., Gearhart, M. D., Cui, Z., Bosnakovski, D., Kim, M., Schennum, N., and Kyba, M. (2016). DUX4 recruits p300/CBP through its C-terminus and induces global H3K27 acetylation changes. Nucleic Acids Res.
Cibulskis, K., Lawrence, M. S., Carter, S. L., Sivachenko, A., Jaffe, D., Sougnez, C., Gabriel, S., Meyerson, M., Lander, E. S., and Getz, G. (2013). Sensitive detection of somatic point mutations in impure and heterogeneous cancer samples. Nat Biotechnol 31, 213-219.
Colaprico, A., Silva, T. C., Olsen, C., Garofano, L., Cava, C., Garolini, D., Sabedot, T. S., Malta, T. M., Pagnotta, S. M., Castiglioni, I., et al. (2016). TCGAbiolinks: an R/Bioconductor package for integrative analysis of TCGA data. Nucleic Acids Res 44, e71-e71.
Das, S., and Chadwick, B. P. (2016). Influence of Repressive Histone and DNA Methylation upon D4Z4 Transcription in Non-Myogenic Cells. PLoS ONE 11, e0160022.
Daxinger, L., Harten, S. K., Oey, H., Epp, T., Isbel, L., Huang, E., Whitelaw, N., Apedaile, A., Sorolla, A., Yong, J., et al. (2013). An ENU mutagenesis screen identifies novel and known genes involved in epigenetic processes in the mouse. Genome Biol 14, R96.
De Iaco, A., Coudray, A., Duc, J., and Trono, D. (2019). DPPA2 and DPPA4 are necessary to establish a 2C-like state in mouse embryonic stem cells. EMBO Rep e47382.
De Iaco, A., Planet, E., Coluccio, A., Verp, S., Duc, J., and Trono, D. (2017). DUX-family transcription factors regulate zygotic genome activation in placental mammals. Nat Genet.
Dvinge, H., Ries, R. E., Hagan, J. O., Stirewalt, D. L., Meshinchi, S., and Bradley, R. K. (2014). Sample processing obscures cancer-specific alterations in leukemic transcriptomes. Proceedings of the National Academy of Sciences 111, 16802-16807.
Eckersley-Maslin, M., Alda-Catalinas, C., Blotenburg, M., Kreibich, E., Krueger, C., and Reik, W. (2019). Dppa2 and Dppa4 directly regulate the Dux-driven zygotic transcriptional program. Genes Dev.
Eidahl, J. O., Giesige, C. R., Domire, J. S., Wallace, L. M., Fowler, A. M., Guckes, S. M., Garwick-Coppens, S. E., Labhart, P., and Harper, S. Q. (2016). Mouse Dux is myotoxic and shares partial functional homology with its human paralog DUX4. Hum Mol Genet 25, 4577-4589.
Eisenhauer, E. A., Therasse, P., Bogaerts, J., Schwartz, L. H., Sargent, D., Ford, R., Dancey, J., Arbuck, S., Gwyther, S., Mooney, M., et al. (2009). New response evaluation criteria in solid tumours: Revised RECIST guideline (version 1.1). European Journal of Cancer 45, 228-247.
Feng, Q., Snider, L., Jagannathan, S., Tawil, R., van der Maarel, S. M., Tapscott, S. J., and Bradley, R. K. (2015). A feedback loop between nonsense-mediated decay and the retrogene DUX4 in facioscapulohumeral muscular dystrophy. Elife 4.
Flicek, P., Ahmed, I., Amode, M. R., Barrell, D., Beal, K., Brent, S., Carvalho-Silva, D., Clapham, P., Coates, G., Fairley, S., et al. (2013). Ensembl 2013. Nucleic Acids Res 41, D48-D55.
Friedman, R. L., Manly, S. P., McMahon, M., Kerr, I. M., and Stark, G. R. (1984). Transcriptional and posttranscriptional regulation of interferon-induced gene expression in human cells. Cell 38, 745-755.
Gabriëls, J., Beckers, M. C., Ding, H., De Vriese, A., Plaisance, S., van der Maarel, S. M., Padberg, G. W., Frants, R. R., Hewitt, J. E., Collen, D., et al. (1999). Nucleotide sequence of the partially deleted D4Z4 locus in a patient with FSHD identifies a putative gene within each 3.3 kb element. Gene 236, 25-32.
Gao, J., Shi, L. Z., Zhao, H., Chen, J., Xiong, L., He, Q., Chen, T., Roszik, J., Bernatchez, C., Woodman, S. E., et al. (2016). Loss of IFN-γ Pathway Genes in Tumor Cells as a Mechanism of Resistance to Anti-CTLA-4 Therapy. Cell 167, 397-404.e399.
Geng, L. N., Tyler, A. E., and Tapscott, S. J. (2011). Immunodetection of human double homeobox 4. Hybridoma (Larchmt) 30, 125-130.
Geng, L. N., Yao, Z., Snider, L., Fong, A. P., Cech, J. N., Young, J. M., van der Maarel, S. M., Ruzzo, W. L., Gentleman, R. C., Tawil, R., et al. (2012). DUX4 activates germline genes, retroelements, and immune mediators: implications for facioscapulohumeral dystrophy. Dev Cell 22, 38-51.
Hanahan, D., and Weinberg, R. A. (2000). The hallmarks of cancer. Cell 100, 57-70.
Hanahan, D., and Weinberg, R. A. (2011). Hallmarks of cancer: the next generation. Cell 144, 646-674.
Hayashi, H., Arao, T., Togashi, Y., Kato, H., Fujita, Y., De Velasco, M. A., Kimura, H., Matsumoto, K., Tanaka, K., Okamoto, I., et al. (2013). The OCT4 pseudogene POU5F1B is amplified and promotes an aggressive phenotype in gastric cancer. Oncogene 34, 199-208.
Haynes, P., Bomsztyk, K., and Miller, D. G. (2018). Sporadic DUX4 expression in FSHD myocytes is associated with incomplete repression by the PRC2 complex and gain of H3K9 acetylation on the contracted D4Z4 allele. Epigenetics Chromatin 11, 47.
Hendrickson, P. G., Doráis, J. A., Grow, E. J., Whiddon, J. L., Lim, J.-W., Wike, C. L., Weaver, B. D., Pflueger, C., Emery, B. R., Wilcox, A. L., et al. (2017). Conserved roles of mouse DUX and human DUX4 in activating cleavage-stage genes and MERVL/HERVL retrotransposons. Nat Genet.
Homey, B., Müller, A., and Zlotnik, A. (2002). Chemokines: agents for the immunotherapy of cancer? Nat. Rev. Immunol. 2, 175-184.
Huber, W., Carey, V. J., Gentleman, R., Anders, S., Carlson, M., Carvalho, B. S., Bravo, H. C., Davis, S., Gatto, L., Girke, T., et al. (2015). Orchestrating high-throughput genomic analysis with Bioconductor. Nat Methods 12, 115-121.
Hugo, W., Zaretsky, J. M., Sun, L., Song, C., Moreno, B. H., Hu-Lieskovan, S., Berent-Maoz, B., Pang, J., Chmielowski, B., Cherry, G., et al. (2016). Genomic and Transcriptomic Features of Response to Anti-PD-1 Therapy in Metastatic Melanoma. Cell 165, 35-44.
Huichalaf, C., Micheloni, S., Ferri, G., Caccia, R., and Gabellini, D. (2014). DNA methylation analysis of the macrosatellite repeat associated with FSHD muscular dystrophy at single nucleotide level. PLoS ONE 9, e115278.
Ikeda, H., Lethé, B., Lehmann, F., van Baren, N., Baurain, J. F., de Smet, C., Chambost, H., Vitale, M., Moretta, A., Boon, T., et al. (1997). Characterization of an antigen that is recognized on a melanoma showing partial HLA loss by CTL expressing an NK inhibitory receptor. Immunity 6, 199-208.
Jagannathan, S., Shadle, S. C., Resnick, R., Snider, L., Tawil, R. N., van der Maarel, S. M., Bradley, R. K., and Tapscott, S. J. (2016). Model systems of DUX4 expression recapitulate the transcriptional profile of FSHD cells. Hum Mol Genet.
Katz, Y., Wang, E. T., Airoldi, E. M., and Burge, C. B. (2010). Analysis and design of RNA sequencing experiments for identifying isoform regulation. Nat Methods 7, 1009-1015.
Kawamura-Saito, M., Yamazaki, Y., Kaneko, K., Kawaguchi, N., Kanda, H., Mukai, H., Gotoh, T., Motoi, T., Fukayama, M., Aburatani, H., et al. (2006). Fusion between CIC and DUX4 up-regulates PEA3 family genes in Ewing-like sarcomas with t(4;19)(q35;q13) translocation. Hum Mol Genet 15, 2125-2137.
Kayisli, U. A., Selam, B., Guzeloglu-Kayisli, O., Demir, R., and Arici, A. (2003). Human chorionic gonadotropin contributes to maternal immunotolerance and endometrial apoptosis by regulating Fas-Fas ligand system. J. Immunol. 171, 2305-2313.
Kim, M.-S., Pinto, S. M., Getnet, D., Nirujogi, R. S., Manda, S. S., Chaerkady, R., Madugundu, A. K., Kelkar, D. S., Isserlin, R., Jain, S., et al. (2014). A draft map of the human proteome. Nature 509, 575-581.
Kosti, I., Jain, N., Aran, D., Butte, A. J., and Sirota, M. (2016). Cross-tissue Analysis of Gene and Protein Expression in Normal and Cancer Tissues. Sci Rep 6, 24799.
Kowaljow, V., Marcowycz, A., Ansseau, E., Conde, C. B., Sauvage, S., Matteotti, C., Arias, C., Corona, E. D., Nuñez, N. G., Leo, O., et al. (2007). The DUX4 gene at the FSHD1A locus encodes a pro-apoptotic protein. Neuromuscul. Disord. 17, 611-623.
Krämer, A., Hochhaus, A., Saussele, S., Reichert, A., Willer, A., and Hehlmann, R. (1998). Cyclin A1 is predominantly expressed in hematological malignancies with myeloid differentiation. Leukemia 12, 893-898.
Kruse, V., Hamann, C., Monecke, S., Cyganek, L., Elsner, L., Hilbscher, D., Walter, L., Streckfuss-Bomeke, K., Guan, K., and Dressel, R. (2015). Human Induced Pluripotent Stem Cells Are Targets for Allogeneic and Autologous Natural Killer (NK) Cells and Killing Is Partly Mediated by the Activating NK Receptor DNAM-1. PLoS ONE 10, e0125544.
Langmead, B., Trapnell, C., Pop, M., and Salzberg, S. L. (2009). Ultrafast and memory-efficient alignment of short DNA sequences to the human genome. Genome Biol 10, R25.
Lee, J. H., Goto, K., Matsuda, C., and Arahata, K. (1995). Characterization of a tandemly repeated 3.3-kbKpnl unit in the facioscapulohumeral muscular dystrophy (FSHD) gene region on chromosome 4q35. Muscle & Nerve 18, S6-S13.
Lemmers, R. J. L. F., Tawil, R., Petek, L. M., Balog, J., Block, G. J., Santen, G. W. E., Amell, A. M., van der Vliet, P. J., Almomani, R., Straasheijm, K. R., et al. (2012). Digenic inheritance of an SMCHD1 mutation and an FSHD-permissive D4Z4 allele causes facioscapulohumeral muscular dystrophy type 2. Nat Genet.
Lemmers, R. J. L. F., van der Vliet, P. J., Klooster, R., Sacconi, S., Camaro, P., Dauwerse, J. G., Snider, L., Straasheijm, K. R., van Ommen, G. J., Padberg, G. W., et al. (2010). A unifying genetic model for facioscapulohumeral muscular dystrophy. Science 329, 1650-1653.
Lemmers, R. J. L. F., Wohlgemuth, M., van der Gaag, K. J., van der Vliet, P. J., van Teijlingen, C. M. M., de Knijff, P., Padberg, G. W., Frants, R. R., and van der Maarel, S. M. (2007). Specific sequence variations within the 4q35 region are associated with facioscapulohumeral muscular dystrophy. Am J Hum Genet 81, 884-894.
Li, B., and Dewey, C. N. (2011). RSEM: accurate transcript quantification from RNA-Seq data with or without a reference genome. BMC Bioinformatics 12, 323.
Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079.
Li, T., Fan, J., Wang, B., Traugh, N., Chen, Q., Liu, J. S., Li, B., and Liu, X. S. (2017). TIMER: A Web Server for Comprehensive Analysis of Tumor-Infiltrating Immune Cells. Cancer Res 77, e108-e110.
Liao, C., Wang, X. Y., Wei, H. Q., Li, S. Q., Merghoub, T., Pandolfi, P. P., and Wolgemuth, D. J. (2001). Altered myelopoiesis and the development of acute myeloid leukemia in transgenic mice overexpressing cyclin A1. Proc Natl Acad Sci USA 98, 6853-6858.
Lilljebjorn, H., Henningsson, R., Hyrenius-Wittsten, A., Olsson, L., Orsmark-Pietras, C., Palffy, von, S., Askmyr, M., Rissler, M., Schrappe, M., Cario, G., et al. (2016). Identification of ETV6-RUNX1-like and DUX4-rearranged subtypes in paediatric B-cell precursor acute lymphoblastic leukaemia. Nat Commun 7, 11790.
Liu, D., Matzuk, M. M., Sung, W. K., Guo, Q., Wang, P., and Wolgemuth, D. J. (1998). Cyclin A1 is required for meiosis in the male mouse. Nat Genet 20, 377-380.
Liu, Y.-F., Wang, B.-Y., Zhang, W.-N., Huang, J.-Y., Li, B.-S., Zhang, M., Jiang, L., Li, J.-F., Wang, M.-J., Dai, Y.-J., et al. (2016). Genomic Profiling of Adult and Pediatric B-cell Acute Lymphoblastic Leukemia. EBioMedicine 8, 173-183.
Livak, K. J., and Schmittgen, T. D. (2001). Analysis of relative gene expression data using real-time quantitative PCR and the 2(-Delta Delta C(T)) Method. Methods 25, 402-408.
Manguso, R. T., Pope, H. W., Zimmer, M. D., Brown, F. D., Yates, K. B., Miller, B. C., Collins, N. B., Bi, K., LaFleur, M. W., Juneja, V. R., et al. (2017). In vivo CRISPR screening identifies Ptpn2 as a cancer immunotherapy target. Nature 547, 413-418.
Meissner, T. B., Li, A., Biswas, A., Lee, K.-H., Liu, Y.-J., Bayir, E., Iliopoulos, D., van den Elsen, P. J., and Kobayashi, K. S. (2010). NLR family member NLRCS is a transcriptional regulator of MHC class I genes. Proceedings of the National Academy of Sciences 107, 13794-13799.
Meyer, L. R., Zweig, A S., Hinrichs, A. S., Karolchik, D., Kuhn, R. M., Wong, M., Sloan, C. A., Rosenbloom, K. R., Roe, G., Rhead, B., et al. (2013). The UCSC Genome Browser database: extensions and updates 2013. Nucleic Acids Res 41, D64-D69.
Monk, M., and Holding, C. (2001). Human embryonic genes re-expressed in cancer cells. Oncogene 20, 8085-8091.
Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nat Methods 12, 453-457.
Ottaviani, A., Rival-Gervier, S., Boussouar, A., Foerster, A. M., Rondier, D., Sacconi, S., Desnuelle, C., Gilson, E., and Magdinier, F. (2009). The D4Z4 macrosatellite repeat acts as a CTCF and A-type lamins-dependent insulator in facio-scapulo-humeral dystrophy. PLoS Genet 5, e1000394.
Pan, D., Kobayashi, A., Jiang, P., Ferrari de Andrade, L., Tay, R. E., Luoma, A. M., Tsoucas, D., Qiu, X., Lim, K., Rao, P., et al. (2018). A major chromatin regulator determines resistance of tumor cells to T cell-mediated killing. Science 359, 770-775.
Patel, S. J., Sanjana, N. E., Kishton, R. J., Eidizadeh, A., Vodnala, S. K., Cam, M., Gartner, J. J., Jia, L., Steinberg, S. M., Yamamoto, T. N., et al. (2017). Identification of essential genes for cancer immunotherapy. Nature 548, 537-542.
Pierce, G. B. (1983). The cancer cell and its control by the embryo. Rous-Whipple Award lecture. Am. J. Pathol. 113, 117-124.
Pollack, S. M., Li, Y., Blaisdell, M. J., Farrar, E. A., Chou, J., Hoch, B. L., Loggers, E. T., Rodler, E., Eary, J. F., Conrad, E. U., et al. (2012). NYESO-1/LAGE-1s and PRAME are targets for antigen specific T cells in chondrosarcoma following treatment with 5-Aza-2-deoxycitabine. PLoS ONE 7, e32165.
Preussner, J., Zhong, J., Sreenivasan, K., Gunther, S., Engleitner, T., Ktinne, C., Glatzel, M., Rad, R., Looso, M., Braun, T., et al. (2018). Oncogenic Amplification of Zygotic Dux Factors in Regenerating p53-Deficient Muscle Stem Cells Defines a Molecular Cancer Subtype. Cell Stem Cell.
Revenkova, E., Eijpe, M., Heyting, C., Gross, B., and Jessberger, R. (2001). Novel meiosis-specific isoform of mammalian SMC1. Mol Cell Biol 21, 6984-6998.
Ribas, A., and Wolchok, J. D. (2018). Cancer immunotherapy using checkpoint blockade. Science 359, 1350-1355.
Rickard, A. M., Petek, L. M., and Miller, D. G. (2015). Endogenous DUX4 Expression in FSHD Myotubes is Sufficient to Cause Cell Death and Disrupts RNA Splicing and Cell Migration Pathways. Hum Mol Genet.
Robinson, M. D., and Oshlack, A. (2010). A scaling normalization method for differential expression analysis of RNA-seq data. Genome Biol 11, R25.
Rodig, S. J., Gusenleitner, D., Jackson, D. G., Gjini, E., Giobbie-Hurder, A., Jin, C., Chang, H., Lovitch, S. B., Horak, C., Weber, J. S., et al. (2018). MHC proteins confer differential sensitivity to CTLA-4 and PD-1 blockade in untreated metastatic melanoma. Sci Transl Med 10, eaar3342.
Rooney, M. S., Shukla, S. A., Wu, C. J., Getz, G., and Hacohen, N. (2015). Molecular and genetic properties of tumors associated with local immune cytolytic activity. Cell 160, 48-61.
Sade-Feldman, M., Jiao, Y. J., Chen, J. H., Rooney, M. S., Barzily-Rokni, M., Eliane, J.-P., Bjorgaard, S. L., Hammond, M. R., Vitzthum, H., Blackmon, S. M., et al. (2017). Resistance to checkpoint blockade therapy through inactivation of antigen presentation. Nat Commun 8, 1136.
Sakaguchi, S., Yamaguchi, T., Nomura, T., and Ono, M. (2008). Regulatory T cells and immune tolerance. Cell 133, 775-787.
Schroder, K., Hertzog, P. J., Ravasi, T., and Hume, D. A. (2004). Interferon-gamma: an overview of signals, mechanisms and functions. J Leukoc Biol 75, 163-189.
Shadle, S. C., Zhong, J. W., Campbell, A. E., Conerly, M. L., Jagannathan, S., Wong, C.-J., Morello, T. D., van der Maarel, S. M., and Tapscott, S. J. (2017). DUX4-induced dsRNA and MYC mRNA stabilization activate apoptotic pathways in human cell models of facioscapulohumeral dystrophy. PLoS Genet 13, e1006658.
Sharma, P., Hu-Lieskovan, S., Wargo, J. A., and Ribas, A. (2017). Primary, Adaptive, and Acquired Resistance to Cancer Immunotherapy. Cell 168, 707-723.
Sheng, W., LaFleur, M. W., Nguyen, T. H., Chen, S., Chakravarthy, A., Conway, J. R., Li, Y., Chen, H., Yang, H., Hsu, P.-H., et al. (2018). LSD1 Ablation Stimulates Anti-tumor Immunity and Enables Checkpoint Blockade. Cell.
Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat Biotechnol.
Smith, H. A., and McNeel, D. G. (2010). The SSX family of cancer-testis antigens as target proteins for tumor therapy. Clin. Dev. Immunol. 2010, 150591-18.
Snider, L., Geng, L. N., Lemmers, R. J. L. F., Kyba, M., Ware, C. B., Nelson, A. M., Tawil, R., Filippova, G. N., van der Maarel, S. M., Tapscott, S. J., et al. (2010). Facioscapulohumeral dystrophy: incomplete suppression of a retrotransposed gene. PLoS Genet 6, e1001181.
Specht, K., Sung, Y.-S., Zhang, L., Richter, G. H. S., Fletcher, C. D., and Antonescu, C. R. (2014). Distinct transcriptional signature and immunoprofile of CIC-DUX4 fusion-positive round cell tumors compared to EWSR1-rearranged Ewing sarcomas: further evidence toward distinct pathologic entities. Genes Chromosomes Cancer 53, 622-633.
Spranger, S., Bao, R., and Gajewski, T. F. (2015). Melanoma-intrinsic β-catenin signalling prevents anti-tumour immunity. Nature 523, 231-235.
Stadler, G., Chen, J. C., Wagner, K., Robin, J. D., Shay, J. W., Emerson, C. P., and Wright, W. E. (2011). Establishment of clonal myogenic cell lines from severely affected dystrophic muscles—CDK4 maintains the myogenic population. Skelet Muscle 1, 12.
Stenman, U.-H., Alfthan, H., and Hotakainen, K. (2004). Human chorionic gonadotropin in cancer. Clin. Biochem. 37, 549-561.
Tanaka, Y., Kawazu, M., Yasuda, T., Tamura, M., Hayakawa, F., Kojima, S., Ueno, T., Kiyoi, H., Naoe, T., and Mano, H. (2018). Transcriptional activities of DUX4 fusions in B-cell acute lymphoblastic leukemia. Haematologica haemato1.2017.183152.
Therneau, T. M., and Grambsch, P. M. (2000). Modeling Survival Data: Extending the Cox Model. (Springer, New York).
Thorsson, V., Gibbs, D. L., Brown, S. D., Wolf, D., Bortone, D. S., Ou Yang, T.-H., Porta-Pardo, E., Gao, G. F., Plaisier, C. L., Eddy, J. A., et al. (2018). The Immune Landscape of Cancer. Immunity 48, 812-830.e814.
Thorvaldsdottir, H., Robinson, J. T., and Mesirov, J. P. (2013). Integrative Genomics Viewer (IGV): high-performance genomics data visualization and exploration. Brief. Bioinformatics 14, 178-192.
Topalian, S. L., Drake, C. G., and Pardoll, D. M. (2015). Immune checkpoint blockade: a common denominator approach to cancer therapy. Cancer Cell 27, 450-461.
Townsend, A., Ohlén, C., Bastin, J., Ljunggren, H. G., Foster, L., and Kane, K. (1989). Association of class I major histocompatibility heavy and light chains induced by viral peptides. Nature 340, 443-448.
Trapnell, C., Pachter, L., and Salzberg, S. L. (2009). TopHat: discovering splice junctions with RNA-Seq. Bioinformatics 25, 1105-1111.
Van Allen, E. M., Miao, D., Schilling, B., Shukla, S. A., Blank, C., Zimmer, L., Sucker, A., Hillen, U., Foppen, M. H. G., Goldinger, S. M., et al. (2015). Genomic correlates of response to CTLA-4 blockade in metastatic melanoma. Science 350, 207-211.
Wagenmakers, E.-J., Lodewyckx, T., Kuriyal, H., and Grasman, R. (2010). Bay esian hypothesis testing for psychologists: a tutorial on the Savage-Dickey method. Cogn Psychol 60, 158-189.
Wang, X., Spandidos, A., Wang, H., and Seed, B. (2012). PrimerBank: a PCR primer database for quantitative gene expression analysis, 2012 update. Nucleic Acids Res 40, D1144-D1149.
Whiddon, J. L., Langford, A. T., Wong, C.-J., Zhong, J. W., and Tapscott, S. J. (2017). Conservation and innovation in the DUX4-family gene network. Nat Genet.
Wickham, H. (2009). ggplot2: elegant graphics for data analysis (Springer New York).
Wickham, H., and Francois, R. dplyr: A Grammar of Data Manipulation.
Wijmenga, C., Frants, R. R., Hewitt, J. E., van Deutekom, J. C., van Geel, M., Wright, T. J., Padberg, G. W., Hofker, M. H., and van Ommen, G. J. (1993). Molecular genetics of facioscapulohumeral muscular dystrophy. Neuromuscul. Disord. 3, 487-491.
Wolchok, J. D., Hoos, A., O'Day, S., Weber, J. S., Hamid, O., Lebbe, C., Maio, M., Binder, M., Bohnsack, O., Nichol, G., et al. (2009). Guidelines for the Evaluation of Immune Therapy Activity in Solid Tumors: Immune-Related Response Criteria. Clin. Cancer Res. 15, 7412-7420.
Yasuda, T., Tsuzuki, S., Kawazu, M., Hayakawa, F., Kojima, S., Ueno, T., Imoto, N., Kohsaka, S., Kunita, A., Doi, K., et al. (2016). Recurrent DUX4 fusions in B cell acute lymphoblastic leukemia of adolescents and young adults. Nat Genet.
Young, J. M., Whiddon, J. L., Yao, Z., Kasinathan, B., Snider, L., Geng, L. N., Balog, J., Tawil, R., van der Maarel, S. M., and Tapscott, S. J. (2013). DUX4 binding to retroelements creates promoters that are active in FSHD muscle and testis. PLoS Genet 9, e1003947.
Young, M. D., Wakefield, M. J., Smyth, G. K., and Oshlack, A. (2010). Gene ontology analysis for RNA-seq: accounting for selection bias. Genome Biol 11, R14.
Zalzman, M., Falco, G., Sharova, L. V., Nishiyama, A., Thomas, M., Lee, S.-L., Stagg, C. A., Hoang, H. G., Yang, H.-T., Indig, F. E., et al. (2010). Zscan4 regulates telomere elongation and genomic stability in ES cells. Nature 464, 858-863.
Zaretsky, J. M., Garcia-Diaz, A., Shin, D. S., Escuin-Ordinas, H., Hugo, W., Hu-Lieskovan, S., Torrejon, D. Y., Abril-Rodriguez, G., Sandoval, S., Barthly, L., et al. (2016). Mutations Associated with Acquired Resistance to PD-1 Blockade in Melanoma. N Engl J Med 375, 819-829.
Zeisel, A., Yitzhaky, A., Bossel Ben-Moshe, N., and Domany, E. (2013). An accessible database for mouse and human whole transcriptome qPCR primers. Bioinformatics 29, 1355-1356.
Zeng, W., de Greef, J. C., Chen, Y.-Y., Chien, R., Kong, X., Gregson, H. C., Winokur, S. T., Pyle, A., Robertson, K. D., Schmiesing, J. A., et al. (2009). Specific loss of histone H3 lysine 9 trimethylation and HPlgamma/cohesin binding at D4Z4 repeats is associated with facioscapulohumeral dystrophy (FSHD). PLoS Genet 5, e1000559.
Zhang, J., McCastlain, K., Yoshihara, H., Xu, B., Chang, Y., Churchman, M. L., Wu, G., Li, Y., Wei, L., Iacobucci, I., et al. (2016). Deregulation of DUX4 and ERG in acute lymphoblastic leukemia. Nat Genet.
Zhang, X., Ding, L., and Sandford, A. J. (2005). Selection of reference genes for gene expression studies in human neutrophils by real-time PCR. BMC Mol Biol 6, 4.
Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based analysis of ChIP-Seq (MACS). Genome Biol 9, R137.

1. A method for treating cancer in a patient, wherein the cancer comprises DUX+ cancer cells, the method comprising administering a therapeutically effective amount of a DUX4 inhibitor to the patient.

2. (canceled)

3. The method of claim 1, wherein the DUX4 inhibitor comprises an antisense oligonucleotide, small interfering RNA (siRNA), short hairpin RNA (shRNA), double-stranded RNA, an antisense oligonucleotide, a ribozyme, or combinations thereof.

4. (canceled)

5. The method of claim 1, wherein the DUX4 is a molecular inhibitor selected from a Bromodomain and Extra-Terminal motif (BET) inhibitor, a CDK7 inhibitor, a Wnt pathway agonist, a beta-2 adrenergic receptor agonist, a p38 inhibitor, a phosphodiesterase (PDE) inhibitor, a cAMP analog, and combinations thereof.

6. (canceled)

7. The method of claim 5, wherein the DUX4 inhibitor comprises a BET inhibitor selected from JQ1, PFI-1, I-BET-762, I-BET-151, RVX-208, CPI-0610, and combinations thereof.

8. The method of claim 5, wherein the DUX4 inhibitor comprises a beta-2 adrenergic agonist selected from formoterol, albuterol, CGP20712, CI118,551, clenbuterol, and combinations thereof.

9. The method of claim 5, wherein the DUX4 inhibitor comprises a PDE inhibitor selected from ibudilast, crisaborole, and combinations thereof.

10. The method of claim 1, wherein the DUX4 inhibitor comprises 8-Bromoadenosine 3′,5′-cyclic monophosphate, a DUX4-s polypeptide, or protein kinase A.

11-12. (canceled)

13. The method of claim 1, wherein the cancer comprises cutaneous squamous-cell carcinoma, non-colorectal and colorectal gastrointestinal cancer, Merkel-cell carcinoma, anal cancer, cervical cancer, hepatocellular cancer, urothelial cancer, melanoma, lung cancer, non-small cell lung cancer, small cell lung cancer, head and neck cancer, kidney cancer, bladder cancer, Hodgkin's lymphoma, pancreatic cancer, or skin cancer.

14-19. (canceled)

20. The method of claim 1, wherein the method further comprises determining the level of expression of DUX4 or a DUX4-regulated gene in a sample from the patient.

21. (canceled)

22. (canceled)

23. The method of claim 1, wherein the patient has been previously treated with a prior cancer therapy and wherein the patient was determined to be resistant, poorly responsive, or non-responsive to the prior cancer therapy.

24-25. (canceled)

26. The method of claim 1, wherein the method further comprises administration of an additional cancer therapy prior to, after, or concurrently with the DUX4 inhibitor.

27. The method of claim 26, wherein the additional therapy comprises chemotherapy, radiation, surgery, or immunotherapy.

28. (canceled)

29. The method of claim 27, wherein the immunotherapy comprises a checkpoint inhibitor therapy.

30. The method of claim 29, wherein the checkpoint inhibitor therapy comprises administering a checkpoint inhibitor selected from an antagonist of CTLA-4, PD-1, PD-L1, PD-L2, LAG-3, TIM-3, VISTA, TIGIT, IDO1, or combinations thereof.

31. The method of claim 29, wherein the checkpoint inhibitor therapy comprises administering a checkpoint inhibitor selected from Ipilmumab, Nivolumab, Pembrolizumab, BMS-936559, MSB0010718C, MPDL3280A, MedI-4736, pidilizumab, AMP-224, RG7446, Atezolizumab, Ipilimumab, Durvalumab, LY3321367 MBG453, TSR-022, JNJ-61610588, and combinations thereof.

32. The method of claim 1, wherein the cancer comprises a solid tumor that comprises DUX4+ cancer cells on the periphery of the solid tumor.

33. (canceled)

34. A method for treating a DUX4+ cancer in a patient, the method comprising administering a checkpoint inhibitor to the patient and administering a DUX4 inhibitor prior to, after, or concurrently with the checkpoint inhibitor.

35. (canceled)

36. The method of claim 34, wherein the DUX4 inhibitor comprises a nucleic acid inhibitor, an antibody, or a molecular inhibitor, wherein the DUX4 inhibitor comprises a nucleic acid inhibitor selected from an antisense oligonucleotide, small interfering RNA (siRNA), short hairpin RNA (shRNA), double-stranded RNA, an antisense oligonucleotide, a ribozyme, and combinations thereof, and wherein the DUX4 molecular inhibitor is selected from a Bromodomain and Extra-Terminal motif (BET) inhibitor, a CDK7 inhibitor, a Wnt pathway agonist, a beta-2 adrenergic receptor agonist, a phosphodiesterase (PDE) inhibitor, a cAMP analog, and combinations thereof.

37-59. (canceled)

60. The method of claim 34, wherein the checkpoint inhibitor is selected from an antagonist of CTLA-4, PD-1, PD-L1, PD-L2, LAG-3, TIM-3, VISTA, TIGIT, IDO1, or combinations thereof.

61. The method of claim 60, wherein the checkpoint inhibitor is selected from selected from Ipilmumab, Nivolumab, Pembrolizumab, BMS-936559, MSB0010718C, MPDL3280A, MedI-4736, pidilizumab, AMP-224, RG7446, Atezolizumab, Ipilimumab, Durvalumab, LY3321367 MBG453, TSR-022, JNJ-61610588, and combinations thereof.

62-63. (canceled)

64. A composition comprising a DUX4 inhibitor and a checkpoint inhibitor.

65-78. (canceled)


SEQUENCE LISTING (102 sequences)

SEQ ID NO: 1 - DNA, 1710 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 2 - DNA, 1574 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 3 - DNA, 8671 bp, Homo sapiens
gggatgcgcgcgcctggggctctcccacagggggctttcgtgagccaggcagcgagggccgcccccgcgctgcagcccagccaggccgcgacggcagagggggtctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacgcctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccggggacggacggggggaggaaggcagggaggaaaagcggtcctcggcctccgggagtagcgggacccccgccctccgggaaaacggtcagcgtccggcgcgggctgagggctgggcccacagccgccgcgccggccggcgcggcacccattcgccccggttccgtggcccagggagtgggcggtttcctccgggacaaaagaccgggactcgggttgccgtcgggttttcacccgcgcggttcacagaccgcacatccccaggctgagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcatggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctccccgttcctcttcgtctctccggccccaccaccaccaccgccaccacgccctcccccaccaccccccccccccccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctgggtggctggctgtccgggcaggccccctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttggcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcgcggagctcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattggtttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatatgtcaaaacgctcctgtagtctgaacctagacaggagttacatcaccttggggatcagtgcagagatacgtgagaattcc

SEQ ID NO: 4 - protein, 160 aa, Unknown
  Description of Unknown DUX4-s sequence
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQV

SEQ ID NO: 5 - DNA, 13 bp, Artificial Sequence
  Description of Artificial Sequence Synthetic oligonucleotide
gtaatccaatcat

SEQ ID NO: 6 - DNA, 19 bp, Artificial Sequence
  Description of Artificial Sequence Synthetic oligonucleotide
gaggtaatccaatcatgga

SEQ ID NO: 7 - DNA, 15 bp, Artificial Sequence
  Description of Artificial Sequence Synthetic oligonucleotide
cgtaatccaatcagc

SEQ ID NO: 8 - DNA, 19 bp, Artificial Sequence
  Description of Artificial Sequence Synthetic oligonucleotide
tgcgtaatccaatcagcgt

SEQ ID NO: 9 - DNA, 63 bp, Artificial Sequence
  Description of Artificial Sequence Synthetic oligonucleotide
cctgtgggaggtaatccaatcatggaggcagcctgtgggaggtaatccaatcatggaggcaga

SEQ ID NO: 10 - DNA, 82 bp, Artificial Sequence
  Description of Artificial Sequence Synthetic oligonucleotide
gcguacgauacctgtgggaggtaatccaatcatggaggcagcctgtgggaggtaatccaatcatggaggcagaaucccaugc

SEQ ID NO: 11 - DNA, 28 bp, Artificial Sequence
  Description of Artificial Sequence Synthetic oligonucleotide
gtgggaggtaatccaatcatggaggcag

SEQ ID NO: 12 - DNA, 28 bp, Artificial Sequence
  Description of Artificial Sequence Synthetic oligonucleotide
cccatgcgtaatccaatcagcgtacgat

SEQ ID NO: 13 - DNA, 34 bp, Artificial Sequence
  Description of Artificial Sequence Synthetic oligonucleotide
cctgtgggaggtaatccaatcatggaggcagcct

SEQ ID NO: 14 - DNA, 40 bp, Artificial Sequence
  Description of Artificial Sequence Synthetic oligonucleotide
gaccctgtgggaggtaatccaatcatggaggcagtttccc

SEQ ID NO: 15 - RNA, 25 nt, Artificial Sequence
  Description of Artificial Sequence Synthetic oligonucleotide
cucucaccgggccuagaccuagaag

SEQ ID NO: 16 - RNA, 25 nt, Artificial Sequence
  Description of Artificial Sequence Synthetic oligonucleotide
ugcgcacugcgcgcaggucuagcca

SEQ ID NO: 17 - RNA, 25 nt, Artificial Sequence
  Description of Artificial Sequence Synthetic oligonucleotide
acugcgcgcaggucuagccaggaag

SEQ ID NO: 18 - RNA, 25 nt, Artificial Sequence
  Description of Artificial Sequence Synthetic oligonucleotide
cggggugcgcacugcgcgcaggucu

SEQ ID NO: 19 - RNA, 30 nt, Artificial Sequence
  Description of Artificial Sequence Synthetic oligonucleotide
ugcgcacugcgcgcaggucuagccaggaag

SEQ ID NO: 20 - RNA, 30 nt, Artificial Sequence
  Description of Artificial Sequence Synthetic oligonucleotide
acugcgcgcaggucuagccaggaagcgggc

SEQ ID NO: 21 - RNA, 25 nt, Artificial Sequence
  Description of Artificial Sequence Synthetic oligonucleotide
acccgaccccgucccaaccccgcgu

SEQ ID NO: 22 - RNA, 25 nt, Artificial Sequence
  Description of Artificial Sequence Synthetic oligonucleotide
gggcauuuuaauauaucucugaacu

SEQ ID NO: 23 - DNA, 19 bp, Artificial Sequence
  Description of Artificial Sequence Synthetic oligonucleotide
ttcctctctccatctctgc

SEQ ID NO: 24 - DNA, 19 bp, Artificial Sequence
  Description of Artificial Sequence Synthetic oligonucleotide
ttgtcccggaggaaaccgc

SEQ ID NO: 25 - DNA, 25 bp, Artificial Sequence
  Description of Artificial Sequence Synthetic oligonucleotide
ttccctgcatgtttccgggtgcccg

SEQ ID NO: 26 - DNA, 19 bp, Artificial Sequence
  Description of Artificial Sequence Synthetic oligonucleotide
cttccctgcatgtttccgg

SEQ ID NO: 27 - DNA, 25 bp, Artificial Sequence
  Description of Artificial Sequence Synthetic oligonucleotide
ctccgtgggagtcttgagtgtgcca

SEQ ID NO: 28 - DNA, 19 bp, Artificial Sequence
  Description of Artificial Sequence Synthetic oligonucleotide
caccccttcatgaatggcg

SEQ ID NO: 29 - DNA, 19 bp, Artificial Sequence
  Description of Artificial Sequence Synthetic oligonucleotide
caggctccaccccttcatg

SEQ ID NO: 30 - DNA, 19 bp, Artificial Sequence
  Description of Artificial Sequence Synthetic oligonucleotide
ttccgctcaaagcaggctc

SEQ ID NO: 31 - DNA, 25 bp, Artificial Sequence
  Description of Artificial Sequence Synthetic oligonucleotide
aaagcgatccttctcaaaggctcgg

SEQ ID NO: 32 - DNA, 19 bp, Artificial Sequence
  Description of Artificial Sequence Synthetic oligonucleotide
cctgcgcgggcgccctgcc

SEQ ID NO: 33 - DNA, 25 bp, Artificial Sequence
  Description of Artificial Sequence Synthetic oligonucleotide
atctctgcccgccttccctcccgcc

SEQ ID NO: 34 - DNA, 25 bp, Artificial Sequence
  Description of Artificial Sequence Synthetic oligonucleotide
agcgcctggcggcggaacgcagacc

SEQ ID NO: 35 - DNA, 25 bp, Artificial Sequence
  Description of Artificial Sequence Synthetic oligonucleotide
gaaacatgcagggaagggtgcaagc

SEQ ID NO: 36 - DNA, 25 bp, Artificial Sequence
  Description of Artificial Sequence Synthetic oligonucleotide
aagactcccacggaggttcagttcc

SEQ ID NO: 37 - DNA, 25 bp, Artificial Sequence
  Description of Artificial Sequence Synthetic oligonucleotide
tgcacagtccggctgaggtgcacgg

SEQ ID NO: 38 - DNA, 25 bp, Artificial Sequence
  Description of Artificial Sequence Synthetic oligonucleotide
agaaggatcgctttccaggcatcgc

SEQ ID NO: 39 - DNA, 25 bp, Artificial Sequence
  Description of Artificial Sequence Synthetic oligonucleotide
ggcgggagggaaggcgggcagagat

SEQ ID NO: 40 - RNA, 19 nt, Artificial Sequence
  Description of Artificial Sequence Synthetic oligonucleotide
gcagagauggagagaggaa

SEQ ID NO: 41 - DNA, 25 bp, Artificial Sequence
  Description of Artificial Sequence Synthetic oligonucleotide
ggtctgcgttccgccgccaggcgct

SEQ ID NO: 42 - DNA, 19 bp, Artificial Sequence
  Description of Artificial Sequence Synthetic oligonucleotide
gcggtttcctccgggacaa

SEQ ID NO: 43 - DNA, 19 bp, Artificial Sequence
  Description of Artificial Sequence Synthetic oligonucleotide
ggacgacggaggcgtgatt

SEQ ID NO: 44 - DNA, 25 bp, Artificial Sequence
  Description of Artificial Sequence Synthetic oligonucleotide
cgggcacccggaaacatgcagggaa

SEQ ID NO: 45 - DNA, 19 bp, Artificial Sequence
  Description of Artificial Sequence Synthetic oligonucleotide
ccggaaacatgcagggaag

SEQ ID NO: 46 - DNA, 25 bp, Artificial Sequence
  Description of Artificial Sequence Synthetic oligonucleotide
gcttgcacccttccctgcatgtttc

SEQ ID NO: 47 - DNA, 19 bp, Artificial Sequence
  Description of Artificial Sequence Synthetic oligonucleotide
gaaatgaacgagagccaca

SEQ ID NO: 48 - DNA, 25 bp, Artificial Sequence
  Description of Artificial Sequence Synthetic oligonucleotide
tggcacactcaagactcccacggag

SEQ ID NO: 49 - DNA, 19 bp, Artificial Sequence
  Description of Artificial Sequence Synthetic oligonucleotide
ccacggaggttcagttcca

SEQ ID NO: 50 - DNA, 25 bp, Artificial Sequence
  Description of Artificial Sequence Synthetic oligonucleotide
ggaactgaacctccgtgggagtctt

SEQ ID NO: 51 - DNA, 19 bp, Artificial Sequence
  Description of Artificial Sequence Synthetic oligonucleotide
accaccaccaccaccacca

SEQ ID NO: 52 - DNA, 19 bp, Artificial Sequence
  Description of Artificial Sequence Synthetic oligonucleotide
cgccattcatgaaggggtg

SEQ ID NO: 53 - DNA, 19 bp, Artificial Sequence
  Description of Artificial Sequence Synthetic oligonucleotide
caugaaggggtggagcctg

SEQ ID NO: 54 - DNA, 25 bp, Artificial Sequence
  Description of Artificial Sequence Synthetic oligonucleotide
ccgcagtgcacagtccggctgaggt

SEQ ID NO: 55 - DNA, 25 bp, Artificial Sequence
  Description of Artificial Sequence Synthetic oligonucleotide
ccgtgcacctcagccggactgtgca

SEQ ID NO: 56 - DNA, 19 bp, Artificial Sequence
  Description of Artificial Sequence Synthetic oligonucleotide
gagcctgctttgagcggaa

SEQ ID NO: 57 - DNA, 25 bp, Artificial Sequence
  Description of Artificial Sequence Synthetic oligonucleotide
ccgagcctttgagaaggatcgcttt

SEQ ID NO: 58 - DNA, 25 bp, Artificial Sequence
  Description of Artificial Sequence Synthetic oligonucleotide
gcgatgcctggaaagcgatccttct

SEQ ID NO: 59 - DNA, 19 bp, Artificial Sequence
  Description of Artificial Sequence Synthetic oligonucleotide
ggcagggcgcccgcgcagg

SEQ ID NO: 60 - DNA, 19 bp, Artificial Sequence
  Description of Artificial Sequence Synthetic oligonucleotide
gcgcaacctctcctagaaa

SEQ ID NO: 61 - DNA, 19 bp, Artificial Sequence
  Description of Artificial Sequence Synthetic oligonucleotide
gggaacacctggctggcta

SEQ ID NO: 62 - DNA, 19 bp, Artificial Sequence
  Description of Artificial Sequence Synthetic oligonucleotide
tacugcgcacgcgcgggtt

SEQ ID NO: 63 - DNA, 19 bp, Artificial Sequence
  Description of Artificial Sequence Synthetic oligonucleotide
cugcctctccaccagccca

SEQ ID NO: 64 - DNA, 19 bp, Artificial Sequence
  Description of Artificial Sequence Synthetic oligonucleotide
caaacctggattagagtta

SEQ ID NO: 65 - DNA, 19 bp, Artificial Sequence
  Description of Artificial Sequence Synthetic oligonucleotide
gatgattagttcagagata

SEQ ID NO: 66 - DNA, 21 bp, Artificial Sequence
  Description of Artificial Sequence Synthetic oligonucleotide
ccgggcatcgccaccagagaa

SEQ ID NO: 67 - DNA, 21 bp, Artificial Sequence
  Description of Artificial Sequence Synthetic oligonucleotide
cagggtccagatttggtttca

SEQ ID NO: 68 - DNA, 21 bp, Artificial Sequence
  Description of Artificial Sequence Synthetic oligonucleotide
caggattcagatctggtttca

SEQ ID NO: 69 - DNA, 21 bp, Artificial Sequence
  Description of Artificial Sequence Synthetic oligonucleotide
caggcgcaacctctcctagaa

SEQ ID NO: 70 - DNA, 19 bp, Artificial Sequence
  Description of Artificial Sequence Synthetic oligonucleotide
gagcctgctttgagcggaa

SEQ ID NO: 71 - DNA, 7760 bp, Unknown
  Description of Unknown D4Z4 sequence
ggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccggggacggacggggggaggaaggcagggaggaaaagcggtcctcggcctccgggagtagcgggacccccgccctccgggaaaacggtcagcgtccggcgcgggctgagggctgggcccacagccgccgcgccggccggcgcggcacccattcgccccggttccgtggcccagggagtgggcggtttcctccgggacaaaagaccgggactcgggttgccgtcgggttttcacccgcgcggttcacagaccgcacatccccaggctgagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcatggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctccccgttcctcttcgtctctccggccccaccaccaccaccgccaccacgccctcccccaccaccccccccccccccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctgggtggctggctgtccgggcaggccccctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttggcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccaccccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcacggagcgcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattgatttacagaacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatgtgtcaaaacgctcctgtagtctgaacctagacaggagttacatcaccttggggatcagtgcagagatacgtgagaattcc

SEQ ID NO: 72 - DNA, 20 bp, Artificial Sequence
  Description of Artificial Sequence Synthetic primer
actgaattcacccccactga

SEQ ID NO: 73 - DNA, 20 bp, Artificial Sequence
  Description of Artificial Sequence Synthetic primer
cctccatgatgctgcttaca

SEQ ID NO: 74 - DNA, 20 bp, Artificial Sequence
  Description of Artificial Sequence Synthetic primer
tcttttcctcttgggcatca

SEQ ID NO: 75 - DNA, 20 bp, Artificial Sequence
  Description of Artificial Sequence Synthetic primer
tagtcccttggttggtgctg

SEQ ID NO: 76 - DNA, 21 bp, Artificial Sequence
  Description of Artificial Sequence Synthetic primer
gtggcattcaaggagtacctc

SEQ ID NO: 77 - DNA, 22 bp, Artificial Sequence
  Description of Artificial Sequence Synthetic primer
tgatggccttcgattctggatt

SEQ ID NO: 78 - DNA, 20 bp, Artificial Sequence
  Description of Artificial Sequence Synthetic primer
cggagaactgccattctttc

SEQ ID NO: 79 - DNA, 20 bp, Artificial Sequence
  Description of Artificial Sequence Synthetic primer
cagccagaatttcacggaag

SEQ ID NO: 80 - DNA, 17 bp, Artificial Sequence
  Description of Artificial Sequence Synthetic primer
cgacgccgcgagccaga

SEQ ID NO: 81 - DNA, 24 bp, Artificial Sequence
  Description of Artificial Sequence Synthetic primer
gcgatgtaatccttgccgtcgtag

SEQ ID NO: 82 - DNA, 17 bp, Artificial Sequence
  Description of Artificial Sequence Synthetic primer
ctaccctgcggagatca

SEQ ID NO: 83 - DNA, 18 bp, Artificial Sequence
  Description of Artificial Sequence Synthetic primer
acagccaggccagcaaca

SEQ ID NO: 84 - DNA, 21 bp, Artificial Sequence
  Description of Artificial Sequence Synthetic primer
ggagacacagaagtacaagcg

SEQ ID NO: 85 - DNA, 22 bp, Artificial Sequence
  Description of Artificial Sequence Synthetic primer
cgtcgtaggcgtactggtcata

SEQ ID NO: 86 - DNA, 20 bp, Artificial Sequence
  Description of Artificial Sequence Synthetic primer
acccaatctcccataggcac

SEQ ID NO: 87 - DNA, 20 bp, Artificial Sequence
  Description of Artificial Sequence Synthetic primer
aggaagggctccacaaactc

SEQ ID NO: 88 - DNA, 20 bp, Artificial Sequence
  Description of Artificial Sequence Synthetic primer
aacctcctccttttccaagc

SEQ ID NO: 89 - DNA, 21 bp, Artificial Sequence
  Description of Artificial Sequence Synthetic primer
gcagtacctgtttagccacga

SEQ ID NO: 90 - DNA, 20 bp, Artificial Sequence
  Description of Artificial Sequence Synthetic primer
gcaagaagaagatcgccaag

SEQ ID NO: 91 - DNA, 20 bp, Artificial Sequence
  Description of Artificial Sequence Synthetic primer
tccaaggggatatccacaga

SEQ ID NO: 92 - DNA, 20 bp, Artificial Sequence
  Description of Artificial Sequence Synthetic primer
acccatcactggactggtgt

SEQ ID NO: 93 - DNA, 20 bp, Artificial Sequence
  Description of Artificial Sequence Synthetic primer
cacatcctcaaagagcctga

SEQ ID NO: 94 - DNA, 20 bp, Artificial Sequence
  Description of Artificial Sequence Synthetic primer
tggaaatcaagtggcaaaaa

SEQ ID NO: 95 - DNA, 18 bp, Artificial Sequence
  Description of Artificial Sequence Synthetic primer
ctgcatgtggacgtggac

SEQ ID NO: 96 - DNA, 21 bp, Artificial Sequence
  Description of Artificial Sequence Synthetic oligonucleotide
aattctccgaacgtgtcacgt

SEQ ID NO: 97 - DNA, 21 bp, Artificial Sequence
  Description of Artificial Sequence Synthetic oligonucleotide
acgggcatgtatcgatacaaa

SEQ ID NO: 98 - DNA, 21 bp, Artificial Sequence
  Description of Artificial Sequence Synthetic oligonucleotide
atggcttgtcatcctgaataa

SEQ ID NO: 99 - DNA, 21 bp, Artificial Sequence
  Description of Artificial Sequence Synthetic oligonucleotide
caacgtcgtcatcaactataa

SEQ ID NO: 100 - DNA, 21 bp, Artificial Sequence
  Description of Artificial Sequence Synthetic oligonucleotide
ctcatcgtggaccacaacata

SEQ ID NO: 101 - DNA, 10 bp, Artificial Sequence
  Description of Artificial Sequence Synthetic oligonucleotide
atatattaaa

SEQ ID NO: 102 - DNA, 19 bp, Artificial Sequence
  Description of Artificial Sequence Synthetic oligonucleotide
tatctctgaactaatcatc